SEQUENCE LISTING (65 sequences)

SEQ ID NO: 1 - protein, 499 aa, Homo sapiens
MTLILTSLLFFGLSLGPRTRVQAENLPKPILWAEPGPVITWHNPVTIWCQGTLEAQGYRLDKEGNSMSRHILKTLESENKVKLSIPSMMWEHAGRYHCYYQSPAGWSEPSDPLELVVTAYSRPTLSALPSPVVTSGVNVTLRCASRLGLGRFTLIEEGDHRLSWTLNSHQHNHGKFQALFPMGPLTFSNRGTFRCYGYENNTPYVWSEPSDPLQLLVSGVSRKPSLLTLQGPVVTPGENLTLQCGSDVGYIRYTLYKEGADGLPQRPGRQPQAGLSQANFTLSPVSRSYGGQYRCYGAHNVSSEWSAPSDPLDILIAGQISDRPSLSVQPGPTVTSGEKVTLLCQSWDPMFTFLLTKEGAAHPPLRLRSMYGAHKYQAEFPMSPVTSAHAGTYRCYGSRSSNPYLLSHPSEPLELVVSGATETLNPAQKKSDSKTAPHLQDYTVENLIRMGVAGLVLLFLGILLFEAQHSQRSPPRCSQEANSRKDNAPFRVVEPWEQI

SEQ ID NO: 2 - protein, 499 aa, Macaca fascicularis
MTPILTTLLCFGLSLGPRTCLQAENLLKPILWAEPGPVIIWKKPVTIWCQGTLEAQEYRLDKEGNSMLRHMLKTLESENKAKFSIPSMMWEHAGRYHCYYQSPAGWSEPSDPLELVVTAYSRPSLSALPSPVVTSGVNVTLRCASRLGLGRFTLIEEGDHRLSWTLDSHQHNHGKFQALFPVGPLTFSNRGTFRCYGYENNTPYVWSEPSDPLQLLVSGVSRKPSLLTLQGPVVAPGDNLTLQCGSDVGYIRYALYKEGGDGLPQRPGQQSQAGLSQASFTLNPVRGSHGGQYRCYGAHNVSSKWSAPSDPLDILIAGQIPDRPSLSVQLGPTVASGEKVTLLCQSWGPMFTFLLAKEGAAHPPLRLRSTYRAQQYQAEFPMSPVTSAHAGTYRCYGSRSSDPYLLSHSSEPLELVVSEATETLNPAQNKSDSKTAPHLQDYTVENLIRMGIAGLVLVFLGILLFEAQQSQRSPTRCSQEVNSREDNAPFRVVEPWEQI

SEQ ID NO: 3-8 - skipped (6 entries)

SEQ ID NO: 9 - protein, 111 aa, synthetic construct
DIVLTQSPASLAVSLGQRATISCRASQSVSTSSYSYMHWYQQKPGQPPKLLIKYASNLESGVPARFSGSGSGTDFTLNIHPVEEEDTATYYCQHSWDIPYTFGGGTKLEIK

SEQ ID NO: 10 - protein, 117 aa, synthetic construct
QIQLVQSGPELKKPGETVKISCKASGYTFTEYPMHWVKQAPGKGFKWMGMIYTDTGEPAYAEEFKGRFAFSLETSASTAYLQINNLKNEDTATYFCVRELGQNFDYWGQGTTLTVSS

SEQ ID NO: 11 - protein, 15 aa, synthetic construct
RASQSVSTSSYSYMH

SEQ ID NO: 12 - protein, 7 aa, synthetic construct
YASNLES

SEQ ID NO: 13 - protein, 9 aa, synthetic construct
QHSWDIPYT

SEQ ID NO: 14 - protein, 5 aa, synthetic construct
EYPMH

SEQ ID NO: 15 - protein, 17 aa, synthetic construct
MIYTDTGEPAYAEEFKG

SEQ ID NO: 16 - protein, 8 aa, synthetic construct
ELGQNFDY

SEQ ID NO: 17 - protein, 7 aa, synthetic construct
GYTFTEY

SEQ ID NO: 18 - protein, 6 aa, synthetic construct
YTDTGE

SEQ ID NO: 19 - protein, 111 aa, synthetic construct
DIVLTQSPASLAVSPGQRATITCRASQSVSTSSYSYMHWYQQKPGQPPKLLIYYASNLESGVPARFSGSGSGTDFTLTINPVEAEDTANYYCQHSWDIPYTFGQGTKLEIK

SEQ ID NO: 20 - protein, 111 aa, synthetic construct
DIVLTQSPASLAVSPGQRATITCRASQSVSTSSYSYMHWYQQKPGQPPKLLIKYASNLESGVPARFSGSGSGTDFTLTINPVEANDTANYYCQHSWDIPYTFGQGTKLEIK

SEQ ID NO: 21 - protein, 111 aa, synthetic construct
DIVMTQSPDSLAVSLGERATINCRASQSVSTSSYSYMHWYQQKPGQPPKLLIYYASNLESGVPDRFSGSGSGTDFTLTISSLQAEDVAVYYCQHSWDIPYTFGQGTKLEIK

SEQ ID NO: 22 - protein, 111 aa, synthetic construct
DIVLTQSPDSLAVSLGERATINCRASQSVSTSSYSYMHWYQQKPGQPPKLLIKYASNLESGVPDRFSGSGSGTDFTLTISSLQAEDVAVYYCQHSWDIPYTFGQGTKLEIK

SEQ ID NO: 23 - protein, 117 aa, synthetic construct
QVQLVQSGSELKKPGASVKVSCKASGYTFTEYPMHWVRQAPGQGLEWMGMIYTDTGEPAYAEEFKGRFVFSLDTSVSTAYLQISSLKAEDTAVYYCARELGQNFDYWGQGTTVTVSS

SEQ ID NO: 24 - protein, 117 aa, synthetic construct
QVQLVQSGSELKKPGASVKVSCKASGYTFTEYPMHWVKQAPGQGLKWMGMIYTDTGEPAYAEEFKGRFVFSLDTSVSTAYLQISSLKAEDTAVYYCARELGQNFDYWGQGTTVTVSS

SEQ ID NO: 25 - protein, 117 aa, synthetic construct
QVQLVQSGSELKKPGASVKVSCKASGYTFTEYPMHWVKQAPGQGLKWMGMIYTDTGEPAYAEEFKGRFVFSLDTSVSTAYLQINSLKAEDTAVYYCARELGQNFDYWGQGTTVTVSS

SEQ ID NO: 26 - protein, 117 aa, synthetic construct
QVQLVQSGAEVKKPGASVKVSCKASGYTFTEYPMHWVRQAPGQRLEWMGMIYTDTGEPAYAEEFKGRVTITRDTSASTAYMELSSLRSEDTAVYYCARELGQNFDYWGQGTTVTVSS

SEQ ID NO: 27 - protein, 117 aa, synthetic construct
QVQLVQSGAEVKKPGASVKVSCKASGYTFTEYPMHWVRQAPGQGLEWMGMIYTDTGEPAYAEEFKGRVTITLDTSASTAYMELSSLRSEDTAVYYCARELGQNFDYWGQGTTVTVSS

SEQ ID NO: 28 - protein, 117 aa, synthetic construct
QVQLVQSGAEVKKPGASVKVSCKASGYTFTEYPMHWVRQAPGQGLKWMGMIYTDTGEPAYAEEFKGRVTITLDTSASTAYMELNSLRSEDTAVYYCARELGQNFDYWGQGTTVTVSS

SEQ ID NO: 29 - protein, 107 aa, synthetic construct [sequence omitted]

SEQ ID NO: 30 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 31 - protein, 330 aa, synthetic construct [sequence omitted]

SEQ ID NO: 32 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 33 - protein, 377 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 34 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 5 aa, synthetic construct
  REGION >5: note = The entire sequence of amino acids 1-5 can be repeated one to five times
GGGGS

SEQ ID NO: 36 - protein, 5 aa, synthetic construct
  REGION >5: note = The entire sequence of amino acids 1-5 can be repeated one to five times
EAAAK

SEQ ID NO: 37 - protein, 5 aa, synthetic construct
  REGION 1..2: note = The "PA" residues can be repeated three to five times.
PAPAP

SEQ ID NO: 38-39 - skipped (2 entries)

SEQ ID NO: 40 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 43 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRYTQKSLSLSPGK

SEQ ID NO: 44 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHTTQKSLSLSPGK

SEQ ID NO: 45 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 50 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 57 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 58 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNIFSCSVMHEALHNHYTQKSLSLSPGK

SEQ ID NO: 59 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRYTQKSLSLSPGK

SEQ ID NO: 60 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRYTQKSLSLSPGK

SEQ ID NO: 61 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNRYTQKSLSLSPGK

SEQ ID NO: 62 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHTTQKSLSLSPGK

SEQ ID NO: 63 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEGLHNHTTQKSLSLSPGK

SEQ ID NO: 64 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEGLHNHTTQKSLSLSPGK

SEQ ID NO: 65 - protein, 6 aa, synthetic construct
HHHHHH

(19) United States
(12) Patent Application Publication
(10) Pub. No.: US 2026/0265373 A1
(43) Pub. Date: Sep. 10, 2026

(54) ILT7-TARGETING ANTIBODIES AND USES THEREOF

(71) Applicant: Inmagene Pte. Ltd., Singapore (SG)

(72) Inventors: Pengcheng Fan, Hangzhou, Zhejiang (CN); Run Lei, Xuhui District, Shanghai (CN); Chongtian Guo, Xuhui District, Shanghai (CN); Xiaotong Wu, Xuhui District, Shanghai (CN); Yiqing Li, Hangzhou, Zhejiang (CN); Steven Xu, Hangzhou, Zhejiang (CN)

(21) Appl. No.: 19/165,438

(22) PCT Filed: Mar. 18, 2024
(86) PCT No.: PCT/CN2024/082288

§ 371 Date: Sep. 15, 2025

(30) Foreign Application Priority Data
Mar. 16, 2023 (WO) PCT/CN2023/082006

(51) Int. Cl. C07K 16/28; A61K 39/00; A61P 37/06

(52) U.S. Cl. CPC C07K 16/2803; A61P 37/06; A61K 2039/505; C07K 2317/14; C07K 2317/24; C07K 2317/33; C07K 2317/41; C07K 2317/52; C07K 2317/565; C07K 2317/732; C07K 2317/76; C07K 2317/92

(57) ABSTRACT

Disclosed herein are anti-ILT7 antibodies and antigen-binding fragments, polynucleotides encoding the antibodies and antigen-binding fragments, and pharmaceutical compositions comprising the antibodies and antigen-binding fragments. Uses of the anti-ILT7 antibodies and antigen-binding fragments described herein in treatment of conditions and disorders associated completement activation are also disclosed.

### CROSS-REFERENCES TO RELATED APPLICATIONS

The present application claims priority to International Patent Application No. PCT/CN2023/082006, filed Mar. 16, 2023, the full disclosure of which is incorporated by reference in its entirety for all purposes.

### REFERENCE TO SEQUENCE LISTING SUBMITTED ELECTRONICALLY

This application incorporates by reference a Sequence Listing as an XML file entitled “110961-1435499-018-001-02PCT” created on Mar. 15, 2024 and having a size of 65,204 bytes.

### FIELD

The present invention relates to molecular biology, cell biology, and immunology. Provided herein include anti-ILT7-antibodies and uses thereof in treating plasmacytoid dendritic cells (pDCs) or Type I Interferon (Type I IFN)-associated immunological disorders.

### BACKGROUND

Plasmacytoid dendrite cells (pDCs), responsible for the production of Type I interferons (IFNs) and pro-inflammatory cytokines, are drivers of both innate and adaptive immune responses. Both pDCs and Type I IFNs are involved in multiple immunological disorders. ILT7, a member of the immunoglobulin-like transcript (ILT) or leukocyte immunoglobulin-like receptor (LIR) gene family, is selectively expressed in pDCs. As such, ILT7-targeting agents that can suppress pDCs-associated Type I IFN release are needed, for example, for treating and preventing autoimmune diseases. However, there has been limited success in the development of such ILT7-targeting agents. The compositions and methods provided herein meet these needs and provide relative advantages.

### SUMMARY

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. Some of the exemplary embodiments of the present invention are discussed below.

Provided herein are antibodies or antigen-binding fragments thereof that specifically bind human ILT7, the antibodies or antigen-binding fragments comprising: (1) as defined by Kabat, (a) a light chain variable region (VL) comprising VL CDR1, VL CDR2, and VL CDR3 having the amino acid sequences of SEQ ID NOs: 11, 12, and 13, respectively; or a variant thereof having up to about 5 amino acid substitutions, additions, and/or deletions in the VL CDRs; and/or (b) a heavy chain variable region (VH) comprising VH CDR1, VH CDR2, and VH CDR3 having the amino acid sequences of SEQ ID NOs: 14, 15, and 16, respectively; or a variant thereof having up to about 5 amino acid substitutions, additions, and/or deletions in the VH CDRs; or (2) as defined by Chothia, (a) a VL comprising VL CDR1, VL CDR2, and VL CDR3 having the amino acid sequences of SEQ ID NOs: 11, 12, and 13, respectively; or a variant thereof having up to about 5 amino acid substitutions, additions, and/or deletions in the VL CDRs; and/or (b) a VH comprising VH CDR1, VH CDR2, and VH CDR3 having the amino acid sequences of SEQ ID NOs: 17, 18, and 16, respectively; or a variant thereof having up to about 5 amino acid substitutions, additions, and/or deletions in the VH CDRs.

In some embodiments, the antibodies or antigen-binding fragments provided herein comprise VL CDR1, VL CDR2, VL CDR3, VH CDR1, VH CDR2 and VH CDR3 having the amino acid sequences of SEQ ID NOs: 11, 12, 13, 14, 15, and 16, respectively, as defined by Kabat. In some embodiments, the antibodies or antigen-binding fragments provided herein comprise VL CDR1, VL CDR2, VL CDR3, VH CDR1, VH CDR2 and VH CDR3 having the amino acid sequences of SEQ ID NOs: 11, 12, 13, 17, 18, and 16, respectively, as defined by Chothia.

Provided herein are also antibodies or antigen-binding fragments thereof that specifically bind human ILT7, the antibodies or antigen-binding fragments comprising: (a) a VL having at least 85%, at least 90%, at least 95%, at least 98%, or 100% sequence identity to the amino acid sequence of SEQ ID NO: 9; and/or (b) a VH having at least 85%, at least 90%, at least 95%, at least 98%, or 100% sequence identity to the amino acid sequence of SEQ ID NO: 10. In some embodiments, the antibodies or antigen-binding fragments provided herein comprise a VL and a VH having the amino acid sequences of SEQ ID NOs: 9 and 10, respectively.

Provided herein are also antibodies or antigen-binding fragments thereof that specifically bind human ILT7, the antibodies or antigen-binding fragments comprising (a) a VL comprising VL CDR1, VL CDR2, and VL CDR3 from a VL having the amino acid sequence of SEQ ID NO: 9; and/or (b) a VH comprising VH CDR1, VH CDR2, and VH CDR3 from a VH having the amino acid sequence of SEQ ID NO: 10.

In some embodiments, the anti-ILT7 antibodies or antigen-binding fragments provided herein are chimeric antibodies or antigen-binding fragments, humanized antibodies or antigen-binding fragments, or human antibodies or antigen-binding fragments.

In some embodiments, the anti-ILT7 antibodies or antigen-binding fragments provided herein are humanized antibodies or antigen-binding fragments. In some embodiments, the humanized anti-ILT7 antibodies or antigen-binding fragments comprise: (a) a VL having at least 85%, at least 90%, at least 95%, at least 98%, or 100% sequence identity to an amino acid sequence selected from the group consisting of SEQ ID NOs: 19-22; and/or (b) a VH having at least 85%, at least 90%, at least 95%, at least 98%, or 100% sequence identity to an amino acid sequence selected from the group consisting of SEQ ID NOs: 23-28.

In some embodiments, the humanized anti-ILT7 antibodies or antigen-binding fragments comprise a VL and a VH having the amino acid sequences of (1) SEQ ID NOs: 19 and 23, respectively; (2) SEQ ID NOs: 19 and 24, respectively; (3) SEQ ID NOs: 19 and 25, respectively; (4) SEQ ID NOs: 19 and 26, respectively; (5) SEQ ID NOs: 19 and 27, respectively; (6) SEQ ID NOs: 19 and 28, respectively; (7) SEQ ID NOs: 20 and 23, respectively; (8) SEQ ID NOs: 20 and 24, respectively; (9) SEQ ID NOs: 20 and 25, respectively; (10) SEQ ID NOs: 20 and 26, respectively; (11) SEQ ID NOs: 20 and 27, respectively; (12) SEQ ID NOs: 20 and 28, respectively; (13) SEQ ID NOs: 21 and 23, respectively; (14) SEQ ID NOs: 21 and 24, respectively; (15) SEQ ID NOs: 21 and 25, respectively; (16) SEQ ID NOs: 21 and 26, respectively; (17) SEQ ID NOs: 21 and 27, respectively; (18) SEQ ID NOs: 21 and 28, respectively; (19) SEQ ID NOs: 22 and 23, respectively; (20) SEQ ID NOs: 22 and 24, respectively; (21) SEQ ID NOs: 22 and 25, respectively; (22) SEQ ID NOs: 22 and 26, respectively; (23) SEQ ID NOs: 22 and 27, respectively; or (24) SEQ ID NOs: 22 and 28, respectively.

In some embodiments, the anti-ILT7 antibodies or antigen-binding fragments provided herein are selected from the group consisting of a Fab, a Fab′, a F(ab′)2, a Fv, a scFv, a (scFv)₂, a single domain antibody (sdAb), and a heavy chain antibody (HCAb). In some embodiments, the anti-ILT7 antibodies or antigen-binding fragments provided herein can be IgG1 antibodies, IgG2 antibodies, IgG3 antibodies, or IgG4 antibodies.

In some embodiments, the anti-ILT7 antibodies provided herein are IgG1 antibodies. In some embodiments, the anti-ILT7 IgG1 antibodies provided herein comprise a light chain constant region (CL) having at least 85% sequence identity to kappa CL (CK; SEQ ID NO: 29). In some embodiments, the anti-ILT7 IgG1 antibodies provided herein comprise a light chain constant region (CL) having at least 85% sequence identity to lambda CL (CX; SEQ ID NO: 30).

In some embodiments, the anti-ILT7 IgG1 antibodies provided herein comprise a heavy chain constant region (CH) having at least 85% sequence identity to an amino acid sequence selected from the group consisting of SEQ ID NOs: 31 and 40-44.

In some embodiments of the anti-ILT7 IgG1 antibodies provided herein, the heavy chain constant region (CH) comprises a wild-type IgG1 CH, or comprises at least one amino acid mutation that enhances ADCC (antibody-dependent cellular cytotoxicity) or ADCP (antibody-dependent cellular phagocytosis) of the antibody. In some embodiments, the CH region of the IgG1 antibodies provided herein has an amino acid substitution at L234, L235, G236, S239, F243, H268, D270, R292, S298, Y300, V305, K326, A330, I332, E333, K334, P396, or any combination thereof, numbered according to the EU Index. In some embodiments, the CH region of the IgG1 antibodies provided herein has an amino acid substitution that is L234Y, L235Q, L235V, G236A, G236W, S239D, S239M, F243L, H268D, D270E, R292P, S298A, Y300L, V305I, K326D, A330M, A330L, I332E, E333A, K334A, K334E, or P396L, or any combination thereof, numbered according to the EU Index. In some embodiments of the anti-ILT7 IgG1 antibodies provided herein, the CH region is modified by amino acid substitutions selected from the group consisting of (i) S298A, E333A, and K334A; (ii) S239D and I332E; (iii) S239D, A330L, and I332E; (iv) G236A; (v) G236A, S239D, and I332E; (vi) G236A, A330L, and I332E; (vii) G236A, S239D, A330L, and I332E; (viii) F243L, R292P, Y300L, V305I, and P396L; (ix) L235V, F243L, R292P, Y300L, and P396L; (x) L234Y, L235Q, G236W, S239M, H268D, D270E, and S298A; and (xi) D270E, K326D, A330M, and K334E, numbered according to the EU Index. In some embodiments, the CH region has an amino acid sequence selected from the group consisting of SEQ ID NOs: 45-64.

In some embodiments of the anti-ILT7 IgG1 antibodies provided herein, the Fc is afucosylated.

Provided herein are also antibodies or antigen-binding fragments that compete with an antibody or antigen-binding fragment described herein for binding to human ILT7.

In some embodiments, the anti-ILT7 antibodies or antigen-binding fragments provided herein are bispecific antibodies or multispecific antibodies.

In some embodiments, the anti-ILT7 antibodies or antigen-binding fragments provided herein are monoclonal antibodies or antigen-binding fragments thereof.

In some embodiments, the anti-ILT7 antibodies or antigen-binding fragments provided herein (1) bind to human ILT7 with a K_{D} of 500 nM or less, as measured by SPR; (2) do not specifically bind to LILR family member LILRA1, LILRA2/ILT1, LILRA3/ILT6, LILRA5/ILT11, LILRA6/ILT8, LILRB1/ILT2, LILRB2/ILT4, LILRB3/ILT5, LILRB4/ILT3, or LILB5; (3) inhibit interferon alpha (IFNα) release by peripheral blood mononuclear cells (PBMCs); (4) selectively bind to plasmacytoid dendritic cells (pDCs) in human PBMC; (5) exhibit natural killer cell (NK)-dependent ADCC activity against ILT7-expressing cells; (6) exhibit neutrophil-dependent ADCC activity against ILT7-expressing cells; or (7) exhibit macrophage-dependent ADCP activity against ILT7-expressing cells; or any combination of (1)-(7).

Also provided herein are anti-ILT7 antibodies or antigen-binding fragments thereof that (1) bind to human ILT7 with a K_{D} of 500 nM or less, as measured by SPR; (2) do not specifically bind to LILR family member LILRA1, LILRA2/ILT1, LILRA3/ILT6, LILRA5/ILT11, LILRA6/ILT8, LILRB1/ILT2, LILRB2/ILT4, LILRB3/ILT5, LILRB4/ILT3, or LILB5; (3) inhibit IFNα release by PBMCs; (4) selectively bind to pDCs in human PBMCs; (5) exhibit NK-dependent ADCC activity against ILT7-expressing cells; (6) exhibit neutrophil-dependent ADCC activity against ILT7-expressing cells; or (7) exhibit macrophage-dependent ADCP activity against ILT7-expressing cells; or any combination of (1)-(7).

In some embodiments, the anti-ILT7 antibodies or antigen-binding fragments provided herein (1) inhibit IFNα release by CpG-stimulated PBMCs in vitro with an EC50 of 1 nM or less; (2) exhibit NK-dependent ADCC activity against ILT7-expressing cells with an EC50 of 0.01 nM or less; (3) exhibit neutrophil-dependent ADCC activity against ILT7-expressing cells with an EC50 of 100 nM or less; (4) exhibit macrophage-dependent ADCP activity against ILT7-expressing cells with an EC50 of 10 nM or less; or (5) exhibit macrophage-dependent ADCP activity against ILT7-expressing cells with a maximum phagocytic index of 20% or higher; or any combination of (1)-(5).

In some embodiments, the anti-ILT7 antibodies or antigen-binding fragments provided herein (1) inhibit IFNα release by PBMCs with an EC50 ranging from 0.01 nM to 0.1 nM; (2) exhibit NK-dependent ADCC activity against ILT7-expressing cells with an EC50 ranging from 0.001 nM to 0.01 nM; (3) exhibit neutrophil-dependent ADCC activity against ILT7-expressing cells with an EC50 ranging from 1 nM to 50 nM; (4) exhibit macrophage-dependent ADCP activity against ILT7-expressing cells with an EC50 ranging from 0.5 nM to 5 nM; or (5) exhibit macrophage-dependent ADCP activity against ILT7-expressing cells with a maximum phagocytic index ranging from 20% to 80%; or any combination of (1)-(5).

In some embodiments, the anti-ILT7 antibodies or antigen-binding fragments provided herein exhibit neutrophil-dependent ADCC activity.

Provided herein are also polynucleotides encoding a polypeptide of the anti-ILT7 antibodies or antigen-binding fragments provided herein. Provided herein are also vectors comprising the polynucleotide described herein.

Provided herein are also host cells comprising the polynucleotide described herein, or the vector described herein. In some embodiments, the host cells described herein (1) overexpress N-acetylglucosaminyltransferase III (GnTIII), (2) lack a-1,6-fucosyltransferase (FUT8), or (3) have a low fucose content, or any combination of (1)-(3).

Provided herein are also methods of making the anti-ILT7 antibodies or antigen-binding fragments described herein, the methods comprising culturing the host cell described herein under conditions that allow expression of the antibody or antibody fragment. In some embodiments, the methods provided herein comprises isolating the antibody from the culture.

Provided herein are also pharmaceutical compositions comprising a therapeutically effective amount of the anti-ILT7 antibodies or antigen-binding fragments described herein and a pharmaceutically acceptable carrier.

Provided herein are also methods of reducing Type I interferon (IFN) in a subject in need thereof, the method comprising administering to the subject a therapeutically effective amount of the anti-ILT7 antibodies or antigen-binding fragments described herein. In some embodiments, the Type I interferon is IFNα.

Provided herein are also methods of suppressing or depleting pDCs in a subject in need thereof, the method comprising administering to the subject a therapeutically effective amount of the anti-ILT7 antibodies or antigen-binding fragments described herein.

Provided herein are also methods of reducing autoimmunity in a subject in need thereof, the method comprising administering to the subject an effective amount of the anti-ILT7 antibodies or antigen-binding fragments described herein.

In some embodiments of the methods provided herein, the subject has an autoimmune disease.

Provided herein are also methods of treating an autoimmune disease associated with Type I IFN or pDCs in a subject in need thereof, the method comprising administering to the subject a therapeutically effective amount of the anti-ILT7 antibodies or antigen-binding fragments described herein.

In some embodiments, the autoimmune disease is systemic lupus erythematosus (SLE).

In some embodiments, the methods provided herein further comprise administering an additional therapy to the subject.

In some embodiments of the methods provided herein, the subject is a human.

In some embodiments, provided herein are uses of the anti-ILT7 antibodies or antigen-binding fragments described herein in reducing Type I IFN. Provided herein are also uses of the anti-ILT7 antibodies or antigen-binding fragments described herein for the preparation of a medicament for reducing Type I IFN. In some embodiments of the uses provided herein, the Type I IFN is IFNα.

In some embodiments, provided herein are uses of the anti-ILT7 antibodies or antigen-binding fragments described herein in suppressing or depleting pDCs. Provided herein are also uses of the anti-ILT7 antibodies or antigen-binding fragments described herein for the preparation of a medicament for suppressing or depleting pDCs.

In some embodiments, provided herein are uses of the anti-ILT7 antibodies or antigen-binding fragments described herein in reducing autoimmunity. In some embodiments, provided herein are uses of the anti-ILT7 antibodies or antigen-binding fragments described herein for the preparation of a medicament for reducing autoimmunity.

In some embodiments, provided herein are uses of the anti-ILT7 antibodies or antigen-binding fragments described herein in treating an autoimmune disease associated with Type I IFN or pDCs. In some embodiments, provided herein are uses of the anti-ILTT7 antibodies or antigen-binding fragments described herein for the preparation of a medicament for treating an autoimmune disease associated with Type I IFN or pDCs. In some embodiments, the autoimmune disease is SLE.

### BRIEF DESCRIPTION OF DRAWINGS

FIG. 1 provides ELISA results showing the binding of ILT7 chimeric antibodies, reference antibody (Tab1) and negative control antibody (hIgG1) to human ILT7 protein.

FIGS. 2A-2B provides flow cytometry results showing the binding of ILT7 chimeric antibodies, reference antibody (Tab1) and negative control antibody (hIgG1) to 293F-human ILT7 cells (FIG. 2A) and CHOK1-cynomolgus ILT7 cells (FIG. 2B).

FIG. 3 provides representative results of IFNα release assay on PBMCs stimulated with CpG. The inhibitory activities of ILT7 chimeric antibodies on IFNα release were measured; results from reference antibody (Tab1) and negative control antibody (hIgG1) were also shown.

FIGS. 4A-4B provide flow cytometry results showing the binding of humanized antibodies of cmAb12 (Hu12) and reference antibody (Tab1) to 293F-human ILT7 cells (FIG. 4A) and CHOK1-cynomolgus ILT7 cells (FIG. 4B).

FIGS. 5A-5B provide representative results of IFN-α release assay on PBMCs stimulated with CpG. The inhibitory activities of four humanized antibodies of emAb12 on IFN-α release were measured, as were for cmAb12, the reference antibody (Tab1) and negative control antibody (hIgG1).

FIG. 6 provides representative results from ELISA measurements of the binding affinities of the hu-cmAb12 antibody, reference antibody (Tab1), and negative control antibody (hIgG1) to human ILT7 protein.

FIGS. 7A-7B provide representative flow cytometry results showing the binding of hu-cmAb12, reference antibody (Tab1), and negative control antibody (isotype) to 293F-human ILT7 cells (FIG. 7A) and CHOK1-cynomolgus ILT7 cells (FIG. 7B).

FIG. 8 provides representative flow cytometry results showing the binding of hu-cmAb12, reference antibody (Tab1) and negative control antibody (Isotype) to cell surface ILT7 on pDCs and other immune cells in human PBMCs.

FIGS. 9A-9B provide representative results from IFNα release assay on PBMCs (from 4 donors) stimulated with CpG (graphs and summary table, respectively). The inhibitory activities of hu-cmAb12 and reference antibody (Tab1) on IFNα release were measured.

FIG. 10 provides representative results of cytotoxic activity assay showing NK cell-dependent ADCC activities of hu-cmAb12, reference antibody (Tab1) and negative control antibody (Isotype).

FIG. 11 provides representative results of cytotoxic activity assay showing neutrophil-dependent ADCC activities of hu-cmAb12, reference antibody (Tab1) and negative control antibody (Isotype).

FIGS. 12A-12B provide representative results of phagocytosis assay showing the macrophage-dependent ADCP activities of hu-cmAb12, reference antibody (Tab1) and negative control antibody (Isotype) in three donors (graphs and summary table, respectively).

FIGS. 13A-13B provides representative results of hematologic cell changes post administration of hu-cmAb12, negative control antibody (Isotype) or PBS infusion in peripheral blood of humanized mice (FIG. 13A) and cynomolgus monkeys (FIG. 13B).

### DETAILED DESCRIPTION

The present disclosure provides novel antibodies, including antigen-binding fragments, that specifically bind ILT7 (e.g., human ILT7). Pharmaceutical compositions comprising a therapeutically effective amount of such antibodies or antigen-binding fragments are also disclosed herein. Also disclosed herein are uses of such pharmaceutical compositions for treating autoimmune diseases associated with plasmacytoid dendritic cells (pDCs) and/or Type I interferon (IFN).

pDCs are a subpopulation of dendritic cells (DC) in the peripheral blood and secondary lymphoid organs. Although they make up only about 0.1 to 0.8% of peripheral blood mononuclear cells (PBMC), these cells are drivers for both innate and adaptive immune responses. pDCs enhance innate immune response because they induce chemokines and myeloid cell recruitment, promote the recruitment of monocytes and their differentiation into antigen-presenting cells (APCs), induce the maturation and activation of dendritic cells, and support the recruitment, activation, and cytotoxicity of natural killer (NK) cells. pDCs also facilitate adaptive immune responses because they promote antigen presentation, support the activation and expansion of antigen-specific CD4+ Th cells, drive CD4+ T cell differentiate to Th2 and Treg cells, promote the survival and activity of CD8+ T cells, and improve B cell survival, maturation, differentiation, and autoantibody production.

Importantly, pDCs are the main source of Type-I IFNs (α/β), which promote the function of NK cells, B cells, T cells, and myeloid DCs. Both pDCs and Type I IFNs are known to be involved in the development of immunological disorders, such as autoimmune diseases. E.g., Annu. Rev. Pathol. Mech. Dis. 2019, 14:369-93; J Immunol 2020, 205:2941-2950; Clinic Rev Allerg Immunol 59, 248-272 (2020); Front. Immunol. 12:713779; Int. J. Mol. Sci. 2021, 22, 4190; Rheumatology 2017; 56:16621675.

Immunoglobulin-Like transcript-7 (ILT7), also known as Leukocyte Immunoglobulin Like Receptor A4 (LIRA4 or LILRA4), or CD85g, is a member of the immunoglobulin-like transcript (ILT) or leukocyte immunoglobulin-like receptor (LIR) gene family. ILT7 contains four immunoglobulin-like extracellular domains and a transmembrane domain. The extracellular portion is important for interacting with the ILT7 ligand, bone marrow stromal cell antigen 2 (BST2), and the transmembrane domain of ILT7 contains a positively charged residue that allows it to bind with FcεRIγ and inhibit pDCs function through an ITAM-mediated signaling pathway.

Full length human ILT7 is a 499-amino acid protein (Uniprot Accession No. P59901, SEQ ID NO: 1), which contains a signal peptide (amino acids 1-23; removed in the mature protein), an extracellular domain (amino acids 24-446), a transmembrane domain (amino acids 447-467), and a cytoplasmic domain (amino acids 468-499). The extracellular domain includes four immunoglobulin-like C2 domains (amino acids 24-118, 123-213, 224-313, and 324-413).

(SEQ ID NO: 1)
MTLILTSLLFFGLSLGPRTRVQAENLPKPILWAEPGPVITWHNPV
TIWCQGTLEAQGYRLDKEGNSMSRHILKTLESENKVKLSIPSMMW
EHAGRYHCYYQSPAGWSEPSDPLELVVTAYSRPTLSALPSPVVTS
GVNVTLRCASRLGLGRFTLIEEGDHRLSWTLNSHQHNHGKFQALF
PMGPLTFSNRGTFRCYGYENNTPYVWSEPSDPLQLLVSGVSRKPS
LLTLQGPVVTPGENLTLQCGSDVGYIRYTLYKEGADGLPQRPGRQ
PQAGLSQANFTLSPVSRSYGGQYRCYGAHNVSSEWSAPSDPLDIL
IAGQISDRPSLSVQPGPTVTSGEKVTLLCQSWDPMFTFLLTKEGA
AHPPLRLRSMYGAHKYQAEFPMSPVTSAHAGTYRCYGSRSSNPYL
LSHPSEPLELVVSGATETLNPAQKKSDSKTAPHLQDYTVENLIRM
GVAGLVLLFLGILLFEAQHSQRSPPRCSQEANSRKDNAPFRVVEP
WEQI

More information about human ILT7 can be found in public databases with the following IDs: HGNC: 15503; NCBI Entrez Gene: 23547; Ensembl: ENSG00000239961; OMIM®: 607517; UniProtKB/Swiss-Prot: Q8IZF0. Two alternatively spliced transcript variants encoding different isoforms are described for the human ILT7 gene (Uniprot NOs: P59901-1, P59901-2).

The sequence of cynomolgus ILT7 is provided below:

(SEQ ID NO: 2)
MTPILTTLLCFGLSLGPRTCLQAENLLKPILWAEPGPVIIWKKPV
TIWCQGTLEAQEYRLDKEGNSMLRHMLKTLESENKAKFSIPSMMW
EHAGRYHCYYQSPAGWSEPSDPLELVVTAYSRPSLSALPSPVVTS
GVNVTLRCASRLGLGRFTLIEEGDHRLSWTLDSHQHNHGKFQALF
PVGPLTFSNRGTFRCYGYENNTPYVWSEPSDPLQLLVSGVSRKPS
LLTLQGPVVAPGDNLTLQCGSDVGYIRYALYKEGGDGLPQRPGQQ
SQAGLSQASFTLNPVRGSHGGQYRCYGAHNVSSKWSAPSDPLDIL
IAGQIPDRPSLSVQLGPTVASGEKVTLLCQSWGPMFTFLLAKEGA
AHPPLRLRSTYRAQQYQAEFPMSPVTSAHAGTYRCYGSRSSDPYL
LSHSSEPLELVVSEATETLNPAQNKSDSKTAPHLQDYTVENLIRM
GIAGLVLVFLGILLFEAQQSQRSPTRCSQEVNSREDNAPFRVVEP
WEQI

ILT7 is selectively expressed on the surface of human pDCs, and not on myeloid DCs or other peripheral blood leukocytes. ILT7 transcripts are minimally detected in most human tissues but are moderately enriched in lymphoid organs, where pDCs reside. Without being bound by theory, the anti-ILT7 antibodies or antigen binding fragments provided herein are useful for reducing pDC activities by NK/neutrophil-mediated ADCC (antibody-dependent cellular cytotoxicity) and/or macrophage-mediated ADCP (antibody-dependent cellular phagocytosis), and are therefore useful in, for example, treating and preventing autoimmune diseases.

Before the present disclosure is further described, it is to be understood that the disclosure is not limited to the particular embodiments set forth herein, and it is also to be understood that the terminology used herein is for the purpose of describing particular embodiments, and is not intended to be limiting.

### A. Definitions

Unless otherwise defined herein, scientific and technical terms used in the present disclosures shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

The term “a” or “an” entity refers to one or more of that entity; for example, “an antibody,” is understood to represent one or more antibodies.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the term “about” is used to indicate that a value includes the inherent variation of error for the device, the method being employed to determine the value, or the variation that exists among the study subjects. The term “about” encompasses the exact number recited. In some embodiments, “about” means within plus or minus 10% of a given value or range. In certain embodiments, “about” means that the variation is ±5%, ±4%, ±3%, ±2%, ±1%, ±0.5%, ±0.2%, or 0.1% of the value to which “about” refers. In some embodiments, “about” means that the variation is ±1%, ±0.5%, ±0.2%, or ±0.1% of the value to which “about” refers.

The term “antibody,” and its grammatical equivalents as used herein refer to an immunoglobulin molecule that recognizes and specifically binds a target, such as a protein, polypeptide, peptide, carbohydrate, polynucleotide, lipid, or a combination of any of the foregoing, through at least one antigen-binding site wherein the antigen-binding site is usually within the variable region of the immunoglobulin molecule. As used herein, the term encompasses intact polyclonal antibodies, intact monoclonal antibodies, single-domain antibodies (sdAbs; e.g., camelid antibodies, alpaca antibodies), single-chain Fv (scFv) antibodies, heavy chain antibodies (HCAbs), light chain antibodies (LCAbs), multispecific antibodies, bispecific antibodies, monospecific antibodies, monovalent antibodies, and any other modified immunoglobulin molecule comprising an antigen-binding site (e.g., dual variable domain immunoglobulin molecules) as long as the antibodies exhibit the desired biological activity. Antibodies also include, but are not limited to, mouse antibodies, camel antibodies, chimeric antibodies, humanized antibodies, and human antibodies. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. Unless expressly indicated otherwise, the term “antibody” as used herein includes an “antigen-binding fragment” of intact antibodies. The term “antigen-binding fragment” as used herein refers to a portion or fragment of an intact antibody that is the antigenic determining variable region of an intact antibody. Examples of antigen-binding fragments include, but are not limited to, Fab, Fab′, F(ab′)2, Fv, linear antibodies, single chain antibody molecules (e.g., scFv), heavy chain antibodies (HCAbs), light chain antibodies (LCAbs), disulfide-linked scFv (dsscFv), diabodies, tribodies, tetrabodies, minibodies, dual variable domain antibodies (DVD), single variable domain antibodies (sdAbs; e.g., camelid antibodies, alpaca antibodies), and single variable domain of heavy chain antibodies (VHH), and bispecific or multispecific antibodies formed from antibody fragments. A “bispecific” antibody is an artificial hybrid antibody having two different antigen binding sites, which recognize and specifically bind two different targets. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The term “humanized antibody” as used herein refers to forms of non-human (e.g., murine) antibodies that are specific immunoglobulin chains, chimeric immunoglobulins, or fragments thereof that contain minimal non-human sequences. Typically, humanized antibodies are human immunoglobulin. In some instances, the Fv framework region residues of a human immunoglobulin are replaced with the corresponding residues in an antibody from a non-human species. In some instances, residues of the CDRs are replaced by residues from the CDRs of a non-human species (e.g., mouse, rat, hamster, camel) that have the desired specificity, affinity, and/or binding capability. The humanized antibody can be further modified by the substitution of additional residues either in the Fv framework region and/or within the replaced non-human residues to refine and optimize antibody specificity, affinity, and/or binding capability. The term “human antibody” as used herein refers to an antibody produced by a human or an antibody having an amino acid sequence corresponding to an antibody produced by a human made using any of the techniques known in the art.

The term “heavy chain” when used in reference to an antibody refers to a polypeptide chain of about 50-70 kDa, wherein the amino-terminal portion includes a variable region of about 120 to 130 or more amino acids and a carboxy-terminal portion that includes a constant region. The constant region can be one of five distinct types, referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while μ and ε contain approximately 550 amino acids. When combined with a light chain, these distinct types of heavy chains give rise to five well known classes of antibodies, IgA, IgD, IgE, IgG and IgM, respectively, including four subclasses of IgG, namely IgG1, IgG2, IgG3 and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids and a carboxy-terminal portion that includes a constant region. The approximate length of a light chain is 211 to 217 amino acids. There are two distinct types, referred to as kappa (κ) of lambda (λ) based on the amino acid sequence of the constant domains. Light chain amino acid sequences are well known in the art. A light chain can be a human light chain.

The term “variable domain” or “variable region” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable domains differ extensively in sequence between different antibodies. The variability in sequence is concentrated in the CDRs while the less variable portions in the variable domain are referred to as framework regions (FR). The CDRs of the light and heavy chains are primarily responsible for the interaction of the antibody with antigen. Numbering of amino acid positions used herein is according to the EU Index, as in Kabat et al. (1991) Sequences of proteins of immunological interest. (U.S. Department of Health and Human Services, Washington, D.C.) 5^{th} ed. A variable region can be a human variable region.

A CDR refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences. CDR regions are well known to those skilled in the art and have been defined by a variety of methods/systems. These systems and/or definitions have been developed and refined over years and include Kabat, Chothia, IMGT, AbM, and Contact. For example, Kabat defines the regions of most hypervariability within the antibody variable (V) domains (Kabat et al, J. Biol. Chem. 252:6609-6616 (1977); Kabat, Adv. Prot. Chem. 32: 1-75 (1978)). The Chothia definition is based on the location of the structural loop regions, which defines CDR region sequences as those residues that are not part of the conserved β-sheet framework, and thus are able to adapt different conformations (Chothia and Lesk, J. Mol. Biol. 196:901-917 (1987)). Both terminologies are well recognized in the art. Additionally, the IMGT system is based on sequence variability and location within the structure of the variable regions. The AbM definition is a compromise between Kabat and Chothia. The Contact definition is based on analyses of the available antibody crystal structures. Software programs (e.g., abYsis) are available and known to those of skill in the art for analysis of antibody sequence and determination of CDRs. The positions of CDRs within a canonical antibody variable domain have been determined by comparison of numerous structures (Al-Lazikani et al, J. Mol. Biol. 273:927-948 (1997); Morea et al, Methods 20:267-279 (2000)). Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra (1997)). Such nomenclature is similarly well known to those skilled in the art.

For example, CDRs defined according to either the Kabat (hypervariable) or Chothia (structural) designations, are set forth in the table below.

| | Kabat¹ | Chothia² | Loop Location |
|---|---|---|---|
| VHCDR1 | 31-35 | 26-32 | linking B and C strands |
| VHCDR2 | 50-65 | 53-55 | linking C′ and C″ strands |
| VHCDR3 | 95-102 | 96-101 | linking F and G strands |
| VLCDR1 | 24-34 | 26-32 | linking B and C strands |
| VLCDR2 | 50-56 | 50-52 | linking C′ and C″ strands |
| VLCDR3 | 89-97 | 91-96 | linking F and G strands |

¹Residue numbering follows the nomenclature of Kabat et al., supra
²Residue numbering follows the nomenclature of Chothia et al., supra

One or more CDRs also can be incorporated into a molecule either covalently or noncovalently to make it an immunoadhesin. An immunoadhesin can incorporate the CDR(s) as part of a larger polypeptide chain, can covalently link the CDR(s) to another polypeptide chain, or can incorporate the CDR(s) noncovalently. The CDRs permit the immunoadhesin to bind to a particular antigen of interest. The CDR regions can be analyzed by, for example, the abysis website (abysis.org).

The terms “epitope” and “antigenic determinant” are used interchangeably herein and refer to the site on the surface of a target molecule to which an antibody or antigen-binding fragment binds, such as a localized region on the surface of an antigen. The target molecule can comprise a protein, a peptide, a nucleic acid, a carbohydrate, or a lipid. An epitope having immunogenic activity is a portion of a target molecule that elicits an immune response in an animal. An epitope of a target molecule having antigenic activity is a portion of the target molecule to which an antibody binds, as determined by any method well known in the art, including, for example, by an immunoassay. Antigenic epitopes need not necessarily be immunogenic. Epitopes often consist of chemically active surface groupings of molecules such as amino acids or sugar side chains and have specific three dimensional structural characteristics as well as specific charge characteristics. The term, “epitope” includes linear epitopes and conformational epitopes. A region of a target molecule (e.g., a polypeptide) contributing to an epitope can be contiguous amino acids of the polypeptide or the epitope can come together from two or more non-contiguous regions of the target molecule. The epitope may or may not be a three-dimensional surface feature of the target molecule. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

The term “specifically binds,” as used herein, means that a polypeptide or molecule interacts more frequently, more rapidly, with greater duration, with greater affinity, or with some combination of the above to the epitope, protein, or target molecule than with alternative substances, including related and unrelated proteins. A binding moiety (e.g., antibody) that specifically binds a target molecule (e.g., antigen) can be identified, for example, by immunoassays, ELISAs, Bio-Layer Interferometry (“BLI”), SPR (e.g., Biacore), or other techniques known to those of skill in the art. Typically, a specific reaction will be at least twice background signal or noise and can be more than 10 times background. See, e.g., Paul, ed., 1989, Fundamental Immunology Second Edition, Raven Press, New York at pages 332-336 for a discussion regarding antibody specificity. A binding moiety that specifically binds a target molecule can bind the target molecule at a higher affinity than its affinity for a different molecule. In some embodiments, a binding moiety that specifically binds a target molecule can bind the target molecule with an affinity that is at least 20 times greater, at least 30 times greater, at least 40 times greater, at least 50 times greater, at least 60 times greater, at least 70 times greater, at least 80 times greater, at least 90 times greater, or at least 100 times greater, than its affinity for a different molecule. In some embodiments, a binding moiety that specifically binds a particular target molecule binds a different molecule at such a low affinity that binding cannot be detected using an assay described herein or otherwise known in the art. In some embodiments, “specifically binds” means, for instance, that a binding moiety binds a molecule target with a K_{D} of about 0.1 mM or less. In some embodiments, “specifically binds” means that a polypeptide or molecule binds a target with a K_{D} of at about 10 μM or less or about 1 μM or less. In some embodiments, “specifically binds” means that a polypeptide or molecule binds a target with a K_{D} of at about 0.1 μM or less, about 0.01 μM or less, or about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a binding moiety (e.g., antibody) that specifically binds a first target may or may not specifically bind a second target. As such, “specific binding” does not necessarily require (although it can include) exclusive binding, i.e., binding to a single target. Thus, a binding moiety (e.g., antibody) can, in some embodiments, specifically bind more than one target. For example, an antibody can, in certain instances, comprise two identical antigen-binding sites, each of which specifically binds the same epitope on two or more proteins. In certain alternative embodiments, an antibody can be bispecific and comprise at least two antigen-binding sites with differing specificities.

The term “binding affinity” as used herein generally refers to the strength of the sum total of noncovalent interactions between a binding moiety and a target molecule (e.g., antigen). The binding of a binding moiety and a target molecule is a reversible process, and the affinity of the binding is typically reported as an equilibrium dissociation constant (K_{D}). K_{D} is the ratio of a dissociation rate (k_{off} or k_{d}) to the association rate (kₒₙ or kₐ). The lower the K_{D} of a binding pair, the higher the affinity. A variety of methods of measuring binding affinity are known in the art, any of which can be used for purposes of the present disclosure. Specific illustrative embodiments include the following. In some embodiments, the “K_{D}” or “K_{D} value” can be measured by assays known in the art, for example by a binding assay. The K_{D} may be measured in a radiolabeled antigen binding assay (RIA) (Chen, et al., (1999) J. Mol Biol 293:865-881). The K_{D} or K_{D} value can also be measured by using biolayer interferometry (BLI) using, for example, the Gator system (Probe Life), or the Octet-96 system (Sartorius AG). The K_{D} or K_{D} value can also be measured by using surface plasmon resonance assays (SPR) by Biacore, using, for example, a BIAcore™-2000 or a BIAcore™-3000 BIAcore, Inc., Piscataway, NJ). The binding affinity can also be quantified with EC₅₀, which is the concentration of ligand at which half of the target is present in the bound state in a binding assay.

The terms “polypeptide,” “peptide,” “protein,” and their grammatical equivalents as used interchangeably herein refer to polymers of amino acids of any length, which can be linear or branched. It can include unnatural or modified amino acids or be interrupted by non-amino acids. A polypeptide, peptide, or protein can also be modified with, for example, disulfide bond formation, glycosylation, lipidation, acetylation, phosphorylation, or any other manipulation or modification.

The term “variant” as used herein in relation to a protein or a polypeptide with particular sequence features (the “reference protein” or “reference polypeptide”) refers to a different protein or polypeptide having one or more (such as, for example, about 1 to about 30, about 1 to about 25, about 1 to about 20, about 1 to about 15, about 1 to about 10, or about 1 to about 5) amino acid substitutions, deletions, and/or additions as compared to the reference protein or reference polypeptide. The changes to an amino acid sequence can be amino acid substitutions. The changes to an amino acid sequence can be conservative amino acid substitutions. The changes to an amino acid sequence can be amino acid deletions. A variant can be a fragment of the reference protein or polypeptide. A functional variant of a protein or polypeptide maintains the basic structural and functional properties of the reference protein or polypeptide.

The terms “polynucleotide,” “nucleic acid,” and their grammatical equivalents as used interchangeably herein refers to a polymer or oligomer of nucleotides of any length. The nucleotides can be deoxyribonucleotides, ribonucleotides, modified nucleotides or bases (such as methylated, hydroxymethylated, or glycosylated), non-natural nucleotides, non-nucleotide building blocks that exhibit similar structure and/or function as natural nucleotides (i.e., “nucleotide analogs”), and/or any substrate that can be incorporated into a polymer by DNA or RNA polymerase. The nucleic acids or polynucleotides can be heterogenous or homogenous in composition, can be isolated from naturally occurring sources, or can be artificially or synthetically produced. In addition, the nucleic acids may be DNA or RNA, or a mixture thereof, and can exist permanently or transitionally in single-stranded or double-stranded form, including homoduplex, heteroduplex, and hybrid states. Nucleic acid structures also include, for instance, a DNA/RNA helix, peptide nucleic acid (PNA), morpholino nucleic acid (see, e.g., Braasch and Corey, Biochemistry, 4(14): 4503-4510 (2002) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, Am. Chem. Soc., 122: 8595-8602 (2000)), and/or a ribozyme.

The terms “identical,” percent “identity,” and their grammatical equivalents as used herein in the context of two or more polynucleotides or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two polynucleotides or polypeptides provided herein are substantially identical, meaning they have at least 70%, at least 75%, at least 80%, at least 85%, at least 90%, and in some embodiments at least 95%, at least 96%, at least 97%, at least 98%, or at least 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

The term “vector,” and its grammatical equivalents as used herein refer to a vehicle that is used to carry genetic material (e.g., a polynucleotide sequence), which can be introduced into a host cell, where it can be replicated and/or expressed. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more polynucleotides are to be co-expressed, both polynucleotides can be inserted, for example, into a single expression vector or in separate expression vectors. For single vector expression, the encoding polynucleotides can be operationally linked to one common expression control sequence or linked to different expression control sequences, such as one inducible promoter and one constitutive promoter. The introduction of polynucleotides into a host cell can be confirmed using methods well known in the art. It is understood by those skilled in the art that the polynucleotides are expressed in a sufficient amount to produce a desired product (e.g., an anti-ILT7 antibody or antigen-binding fragment as described herein), and it is further understood that expression levels can be optimized to obtain sufficient expression using methods well known in the art.

As used herein, the term “encode” and its grammatical equivalents refer to the inherent property of specific sequences of nucleotides in a polynucleotide or a nucleic acid, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein. Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

A polypeptide, peptide, protein, antibody, polynucleotide, vector, cell, or composition which is “isolated” is a polypeptide, peptide, protein, antibody, polynucleotide, vector, cell, or composition which is in a form not found in nature. Isolated polypeptides, peptides, proteins, antibodies, polynucleotides, vectors, cells, or compositions include those which have been purified to a degree that they are no longer in a form in which they are found in nature. In some embodiments, a polypeptide, peptide, protein, antibody, polynucleotide, vector, cell, or composition which is isolated is substantially pure.

The term “treat” and its grammatical equivalents as used herein in connection with a disease or a condition, or a subject having a disease or a condition refer to an action that suppresses, eliminates, reduces, and/or ameliorates a symptom, the severity of the symptom, and/or the frequency of the symptom associated with the disease or disorder being treated.

The term “administer” and its grammatical equivalents as used herein refer to the act of delivering, or causing to be delivered, a therapeutic or a pharmaceutical composition to the body of a subject by a method described herein or otherwise known in the art. The therapeutic can be a compound, a polypeptide, an antibody, a cell, or a population of cells. Administering a therapeutic or a pharmaceutical composition includes prescribing a therapeutic or a pharmaceutical composition to be delivered into the body of a subject. Exemplary forms of administration include oral dosage forms, such as tablets, capsules, syrups, suspensions; injectable dosage forms, such as intravenous (IV), intramuscular (IM), or intraperitoneal (IP); transdermal dosage forms, including creams, jellies, powders, or patches; buccal dosage forms; inhalation powders, sprays, suspensions, and rectal suppositories.

The terms “effective amount,” “therapeutically effective amount,” and their grammatical equivalents as used herein refer to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to the subject. The therapeutically effective amount can be ascertained by measuring relevant physiological effects. The exact amount required vary from subject to subject, depending on the age, weight, and general condition of the subject, the severity of the condition being treated, the judgment of the clinician, and the like. An appropriate “effective amount” in any individual case can be determined by one of ordinary skill in the art using routine experimentation.

The term “pharmaceutically acceptable carrier” or “pharmaceutically acceptable excipient” refers to a material that is suitable for drug administration to an individual along with an active agent without causing undesirable biological effects or interacting in a deleterious manner with any of the other components of the pharmaceutical composition.

The term “subject” as used herein refers to any animal (e.g., a mammal), including, but not limited to, humans, non-human primates, canines, felines, rodents, and the like, which is to be the recipient of a particular treatment. A subject can be a human. A subject can have a particular disease or condition.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, and 6. This applies regardless of the breadth of the range.

Exemplary genes and polypeptides are described herein with reference to GenBank numbers, GI numbers and/or SEQ ID NOs. It is understood that one skilled in the art can readily identify homologous sequences by reference to sequence sources, including but not limited to GenBank (ncbi.nlm.nih.gov/genbank/) and EMBL (embl.org/).

### B. Anti-ILT7 Antibodies and Antigen-Binding Fragments

Provided herein are antibodies or antigen-binding fragments thereof that specifically bind ILT7 (e.g., human ILT7). In some embodiments, provided herein are anti-ILT7 antibodies. In some embodiments, the antibody is an IgA, IgD, IgE, IgG, or IgM antibody. In some embodiments, the antibody is an IgA antibody. In some embodiments, the antibody is an IgD antibody. In some embodiments, the antibody is an IgE antibody. In some embodiments, the antibody is an IgG antibody. In some embodiments, the antibody is an IgM antibody. In some embodiments, the antibodies provided herein can be an IgG1 antibody, an IgG2 antibody, an IgG3 antibody, or an IgG4 antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody.

In some embodiments, provided herein are antigen-binding fragments of an anti-ILT7 antibody. In some embodiments, antigen-binding fragments provided herein can be a single domain antibody (sdAb), a heavy chain antibody (HCAb), a Fab, a Fab′, a F(ab′)2, a Fv, a single-chain variable fragment (scFv), or a (scFv)₂. In some embodiments, the antigen-binding fragment of an anti-ILT7 antibody is a single domain antibody (sdAb). In some embodiments, the antigen-binding fragment of an anti-ILT7 antibody is a heavy chain antibody (HCAb). In some embodiments, the antigen-binding fragment of an anti-ILT7 antibody is a Fab. In some embodiments, the antigen-binding fragment of an anti-ILT7 antibody is a Fab′. In some embodiments, the antigen-binding fragment of an anti-ILT7 antibody is a F(ab′)2. In some embodiments, the antigen-binding fragment of an anti-ILT7 antibody is a Fv. In some embodiments, the antigen-binding fragment of an anti-ILT7 antibody is a scFv. In some embodiments, the antigen-binding fragment of an anti-ILT7 antibody is a disulfide-linked scFv [(scFv)₂]. In some embodiments, the antigen-binding fragment of an anti-ILT7 antibody is a diabody (dAb).

In some embodiments, the anti-ILT7 antibodies or antigen-binding fragments provided herein comprise recombinant antibodies or antigen-binding fragments. In some embodiments, the anti-ILT7 antibodies or antigen-binding fragments provided herein comprise monoclonal antibodies or antigen-binding fragments. In some embodiments, the anti-ILT7 antibodies or antigen-binding fragments provided herein comprise polyclonal antibodies or antigen-binding fragments. In some embodiments, the anti-ILT7 antibodies or antigen-binding fragments provided herein comprise camelid (e.g., camels, dromedary and llamas) antibodies or antigen-binding fragments. In some embodiments, the anti-ILT7 antibodies or antigen-binding fragments provided herein comprise chimeric antibodies or antigen-binding fragments. In some embodiments, the anti-ILT7 antibodies or antigen-binding fragments provided herein comprise humanized antibodies or antigen-binding fragments. In some embodiments, the anti-ILT7 antibodies or antigen-binding fragments provided herein comprise human antibodies or antigen-binding fragments. In some embodiments, provided herein are anti-ILT7 human scFvs.

In some embodiments, the anti-ILT7 antibodies or antigen-binding fragments provided herein are isolated. In some embodiments, the anti-ILT7 antibodies or antigen-binding fragments provided herein are substantially pure.

In some embodiments, the anti-ILT7 antibody or antigen-binding fragment provided herein comprises a multispecific antibody or antigen-binding fragment. In some embodiments, the anti-ILT7 antibody or antigen-binding fragment provided herein comprises a bispecific antibody or antigen-binding fragment. In some embodiments, the bispecific antibody or antigen-binding fragment comprises an anti-ILT7 antibody or antigen-binding fragment provided herein. In some embodiments, the bispecific antibody or antigen-binding fragment comprises an anti-ILT7 scFv provided herein.

In some embodiments, the anti-ILT7 antibody or antigen-binding fragment provided herein comprises a monovalent antigen-binding site. In some embodiments, an anti-ILT7 antibody or antigen-binding fragment comprises a monospecific binding site. In some embodiments, an anti-ILT7 antibody or antigen-binding fragment comprises a bivalent binding site.

In some embodiments, an anti-ILT7 antibody or antigen-binding fragment is a monoclonal antibody or antigen-binding fragment. Monoclonal antibodies can be prepared by any method known to those of skill in the art. One exemplary approach is screening protein expression libraries, e.g., phage or ribosome display libraries. Phage display is described, for example, in Ladner et al., U.S. Pat. No. 5,223,409; Smith (1985) Science 228:1315-1317; and WO 92/18619. In some embodiments, recombinant monoclonal antibodies are isolated from phage display libraries expressing variable regions or CDRs of a desired species. Screening of phage libraries can be accomplished by various techniques known in the art.

In some embodiments, a monoclonal antibody is modified by using recombinant DNA technology to generate alternative antibodies. In some embodiments, the constant domains of the light chain and heavy chain of a mouse monoclonal antibody are replaced with the constant regions of a human antibody to generate a chimeric antibody. In some embodiments, the constant regions are truncated or removed to generate a desired antibody fragment of a monoclonal antibody. In some embodiments, site-directed or high-density mutagenesis of the variable region(s) is used to optimize specificity and/or affinity of a monoclonal antibody.

In some embodiments, provided herein is the anti-ILT7 antibody clone Ab12. The sequence features are described below. The specific CDR sequences defined herein are generally based on either Kabat or Chothia definition. However, it is understood that a general reference to a heavy chain CDR or CDRs and/or a light chain CDR or CDRs of a specific antibody encompass all CDR definitions as known to those of skill in the art.

TABLE 1
Amino acid sequences of light chain variable
region CDRs (VL CDRs) of Ab12

| | VL CDR1 | VL CDR2 | VL CDR3 |
|---|---|---|---|
| Kabat | RASQSVST | YASN | QHSWD |
| | SSYSYMH | LES | IPYT |
| | (SEQ | (SEQ | (SEQ |
| | ID | ID | ID |
| | NO: | NO: | NO: |
| | 11) | 12) | 13) |
| Chothia | RASQSVST | YASN | QHSWD |
| | SSYSYMH | LES | IPYT |
| | (SEQ | (SEQ | (SEQ |
| | ID | ID | ID |
| | NO: | NO: | NO: |
| | 11) | 12) | 13) |

TABLE 2
Amino acid sequences of heavy chain variable
region CDRs (VH CDRs) of Ab12

| Sequence | VH CDR1 | VH CDR2 | VH CDR3 |
|---|---|---|---|
| Kabat | EYPMH | MIYTDTGE | ELGQNFDY |
| | (SEQ | PAYAEEFKG | (SEQ |
| | ID | (SEQ | ID |
| | NO: | ID | NO: |
| | 14) | NO: | 16) |
| | | 15) | |
| Chothia | GYTFTEY | YTDTGE | ELGQNFDY |
| | (SEQ | (SEQ | (SEQ |
| | ID | ID | ID |
| | NO: | NO: | NO: |
| | 17) | 18) | 16) |

In some embodiments, anti-ILT7 antibodies or antigen-binding fragments provided herein comprise one, two, three, four, five, and/or six CDRs of any one of the antibodies described herein. In some embodiments, anti-ILT7 antibodies or antigen-binding fragments provided herein comprise a light chain variable region (VL) comprising one, two, and/or three, light chain CDRs (VL CDRs) from Table 1. In some embodiments, anti-ILT7 antibodies or antigen-binding fragments provided herein comprise a heavy chain variable region (VH) comprising one, two, and/or three heavy chain CDRs (VH CDRs) from Table 2. In some embodiments, anti-ILT7 antibodies or antigen-binding fragments provided herein comprise one, two, and/or three VL CDRs from Table 1 and one, two, and/or three VH CDRs from Table 2.

In some embodiments, provided herein are antibodies or antigen-binding fragments thereof that specifically bind ILT7, comprising a VL comprising (1) a VL CDR1 having the amino acid sequence of SEQ ID NO: 11; (2) a VL CDR2 having the amino acid sequence of SEQ ID NO: 12; and/or (3) a VL CDR3 having the amino acid sequence of SEQ ID NO: 13; or a variant thereof having up to about 3, about 5, about 8, about 10, about 12, or about 15 amino acid substitutions, additions, and/or deletions in the VL CDRs; and/or a VH comprising (1) a VH CDR1 having an amino acid sequence selected from the group consisting of SEQ ID NO: 14 or 17; (2) a VH CDR2 having the amino acid sequence of SEQ ID NO: 15 or 18; and/or (3) a VH CDR3 having the amino acid sequence of SEQ ID NO: 16; or a variant thereof having up to about 3, about 5, about 8, about 10, about 12, or about 15 amino acid substitutions, additions, and/or deletions in the VH CDRs.

In some embodiments, provided herein are antibodies or antigen-binding fragments thereof that specifically bind ILT7, comprising a VL comprising (1) a VL CDR1 having the amino acid sequence of SEQ ID NO: 11; (2) a VL CDR2 having the amino acid sequence of SEQ ID NO: 12; or (3) a VL CDR3 having the amino acid sequence of SEQ ID NO: 13; or a variant thereof having up to about 3, about 5, about 8, about 10, about 12, or about 15 amino acid substitutions, additions, and/or deletions in the VL CDR. In some embodiments, the variant has up to about 5 amino acid substitutions, additions, and/or deletions in the VL CDR. In some embodiments, provided herein are antibodies or antigen-binding fragments thereof that specifically bind ILT7, comprising a VL comprising (1) a VL CDR1 having the amino acid sequence of SEQ ID NO: 11; (2) a VL CDR2 having the amino acid sequence of SEQ ID NO: 12; and (3) a VL CDR3 having the amino acid sequence of SEQ ID NO: 13; or a variant thereof having up to about 3, about 5, about 8, about 10, about 12, or about 15 amino acid substitutions, additions, and/or deletions in the VL CDRs. In some embodiments, the variant has up to about 5 amino acid substitutions, additions, and/or deletions in the VL CDRs.

In some embodiments, provided herein are antibodies or antigen-binding fragments thereof that specifically bind ILT7 having a VL, wherein the VL comprises VL CDR1, CDR2 and CDR3 having the amino acid sequences of SEQ ID NOs: 11, 12 and 13, respectively, as defined by Kabat; or a variant thereof having up to about 3, about 5, about 8, about 10, about 12, or about 15 amino acid substitutions, additions, and/or deletions in the VL CDRs. In some embodiments, the variant has up about 5 amino acid substitutions, additions, and/or deletions in the VL CDRs. In some embodiments, provided herein are antibodies or antigen-binding fragments thereof that specifically bind ILT7 having a VL, wherein the VL comprises VL CDR1, CDR2 and CDR3 having the amino acid sequences of SEQ ID NOs: 11, 12 and 13, respectively, as defined by Chothia; or a variant thereof having up to about 3, about 5, about 8, about 10, about 12, or about 15 amino acid substitutions, additions, and/or deletions in the VL CDRs. In some embodiments, the variant has up about 5 amino acid substitutions, additions, and/or deletions in the VL CDRs.

In some embodiments, provided herein are antibodies or antigen-binding fragments thereof that specifically bind ILT7 comprising a VH comprising (1) a VH CDR1 having an amino acid sequence selected from the group consisting of SEQ ID NO: 14 or 17; (2) a VH CDR2 having the amino acid sequence of SEQ ID NO: 15 or 18; or (3) a VH CDR3 having an amino acid sequence selected from the group consisting of SEQ ID NO: 16; or a variant thereof having up to about 3, about 5, about 8, about 10, about 12, or about 15 amino acid substitutions, additions, and/or deletions in the VH CDR. In some embodiments, the variant has up about 5 amino acid substitutions, additions, and/or deletions in the VH CDR. In some embodiments, provided herein are antibodies or antigen-binding fragments thereof that specifically bind ILT7 comprising a VH comprising (1) a VH CDR1 having an amino acid sequence selected from the group consisting of SEQ ID NO: 14 or 17; (2) a VH CDR2 having the amino acid sequence of SEQ ID NO: 15 or 18; and (3) a VH CDR3 having an amino acid sequence selected from the group consisting of SEQ ID NO: 16; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs.

In some embodiments, provided herein are antibodies or antigen-binding fragments thereof that specifically bind ILT7 having a VH, wherein the VH comprises VH CDR1, CDR2 and CDR3 having the amino acid sequences of SEQ ID NOs: 14, 15, and 16, respectively, as defined by Kabat; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs. In some embodiments, provided herein are antibodies or antigen-binding fragments thereof that specifically bind ILT7 having a VH, wherein the VH comprises VH CDR1, CDR2 and CDR3 having the amino acid sequences of SEQ ID NOs: 17, 18, and 16, respectively; as defined by Chothia; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs.

In some embodiments, provided herein are antibodies or antigen-binding fragments thereof that specifically bind ILT7, comprising, as defined by Kabat, (a) a VL comprising VL CDR1, VL CDR2, and VL CDR3 having the amino acid sequences of SEQ ID NOs: 11, 12, and 13, respectively; or a variant thereof having up to about 3, about 5, about 8, about 10, about 12, or about 15 amino acid substitutions, additions, and/or deletions in the VL CDRs; and/or (b) a VH comprising VH CDR1, VH CDR2, and VH CDR3 having the amino acid sequences of SEQ ID NOs: 14, 15, and 16, respectively; or a variant thereof having up to having up to about 3, about 5, about 8, about 10, about 12, or about 15 amino acid substitutions, additions, and/or deletions in the VH CDRs.

In some embodiments, provided herein are antibodies or antigen-binding fragments thereof that specifically bind ILT7 comprising VL CDR1, VL CDR2, VL CDR3, VH CDR1, VH CDR2 and VH CDR3, having the amino acid sequences of SEQ ID NOs: 11, 12, 13, 14, 15, and 16, respectively, as defined by Kabat, or a variant thereof having up to having up to about 3, about 5, about 8, about 10, about 12, or about 15 amino acid substitutions, additions, and/or deletions in the CDRs.

In some embodiments, provided herein are antibodies or antigen-binding fragments thereof that specifically bind ILT7, comprising, as defined by Chothia (a) a VL comprising VL CDR1, VL CDR2, and VL CDR3 having the amino acid sequences of SEQ ID NOs: 11, 12, and 13, respectively; or a variant thereof having up to about 3, about 5, about 8, about 10, about 12, or about 15 amino acid substitutions, additions, and/or deletions in the VL CDRs; and/or (b) a VH comprising VH CDR1, VH CDR2, and VH CDR3 having the amino acid sequences of NOs: 17, 18, and 16, respectively; or a variant thereof having up to about 3, about 5, about 8, about 10, about 12, or about 15 amino acid substitutions, additions, and/or deletions in the VH CDRs.

In some embodiments, provided herein are antibodies or antigen-binding fragments thereof that specifically bind ILT7 comprising VL CDR1, VL CDR2, VL CDR3, VH CDR1, VH CDR2 and VH CDR3, having the amino acid sequences of SEQ ID NOs: 11, 12, 13, 17, 18, and 16, respectively, as defined by Chothia, or a variant thereof having up to having up to about 3, about 5, about 8, about 10, about 12, or about 15 amino acid substitutions, additions, and/or deletions in the CDRs.

TABLE 3
Amino acid sequences of light chain variable region (VL) and heavy chain variable
region (VH) of chimeric antibody Ab12 (cmAb12) and the humanized VLs (hu-VL) and VHs
(hu-VH).

| Ab domain | Sequence |
|---|---|
| cmAb12 VL | DIVLTQSPASLAVSLGQRATISCRASQSVSTSSYSYMHWYQQKPGQPPKLLIKYA |
| | SNLESGVPARFSGSGSGTDFTLNIHPVEEEDTATYYCQHSWDIPYTFGGGTKLEI |
| | K (SEQ ID NO: 9) |
| cmAb12 VH | QIQLVQSGPELKKPGETVKISCKASGYTFTEYPMHWVKQAPGKGFKWMGMIYT |
| | DTGEPAYAEEFKGRFAFSLETSASTAYLQINNLKNEDTATYFCVRELGQNFDYW |
| | GQGTTLTVSS (SEQ ID NO: 10) |
| Hu-VL1 | DIVLTQSPASLAVSPGQRATITCRASQSVSTSSYSYMHWYQQKPGQPPKLLIYYA |
| | SNLESGVPARFSGSGSGTDFTLTINPVEAEDTANYYCQHSWDIPYTFGQGTKLEI |
| | K (SEQ ID NO: 19) |
| Hu-VL2 | DIVLTQSPASLAVSPGQRATITCRASQSVSTSSYSYMHWYQQKPGQPPKLLIKYA |
| | SNLESGVPARFSGSGSGTDFTLTINPVEANDTANYYCQHSWDIPYTFGQGTKLEI |
| | K (SEQ ID NO: 20) |
| Hu-VL3 | DIVMTQSPDSLAVSLGERATINCRASQSVSTSSYSYMHWYQQKPGQPPKLLIYY |
| | ASNLESGVPDRFSGSGSGTDFTLTISSLQAEDVAVYYCQHSWDIPYTFGQGTKLE |
| | IK (SEQ ID NO: 21) |
| Hu-VL4 | DIVLTQSPDSLAVSLGERATINCRASQSVSTSSYSYMHWYQQKPGQPPKLLIKYA |
| | SNLESGVPDRFSGSGSGTDFTLTISSLQAEDVAVYYCQHSWDIPYTFGQGTKLEI |
| | K (SEQ ID NO: 22) |
| Hu-VH1 | QVQLVQSGSELKKPGASVKVSCKASGYTFTEYPMHWVRQAPGQGLEWMGMIY |
| | TDTGEPAYAEEFKGRFVFSLDTSVSTAYLQISSLKAEDTAVYYCARELGQNFDY |
| | WGQGTTVTVSS (SEQ ID NO: 23) |
| Hu-VH2 | QVQLVQSGSELKKPGASVKVSCKASGYTFTEYPMHWVKQAPGQGLKWMGMI |
| | YTDTGEPAYAEEFKGRFVFSLDTSVSTAYLQISSLKAEDTAVYYCARELGQNED |
| | YWGQGTTVTVSS (SEQ ID NO: 24) |
| Hu-VH3 | QVQLVQSGSELKKPGASVKVSCKASGYTFTEYPMHWVKQAPGQGLKWMGMI |
| | YTDTGEPAYAEEFKGRFVFSLDTSVSTAYLQINSLKAEDTAVYYCARELGQNFD |
| | YWGQGTTVTVSS (SEQ ID NO: 25) |
| Hu-VH4 | QVQLVQSGAEVKKPGASVKVSCKASGYTFTEYPMHWVRQAPGQRLEWMGMI |
| | YTDTGEPAYAEEFKGRVTITRDTSASTAYMELSSLRSEDTAVYYCARELGQNED |
| | YWGQGTTVTVSS (SEQ ID NO: 26) |
| Hu-VH5 | QVQLVQSGAEVKKPGASVKVSCKASGYTFTEYPMHWVRQAPGQGLEWMGMI |
| | YTDTGEPAYAEEFKGRVTITLDTSASTAYMELSSLRSEDTAVYYCARELGQNFD |
| | YWGQGTTVTVSS (SEQ ID NO: 27) |
| Hu-VH6 | QVQLVQSGAEVKKPGASVKVSCKASGYTFTEYPMHWVRQAPGQGLKWMGMI |
| | YTDTGEPAYAEEFKGRVTITLDTSASTAYMELNSLRSEDTAVYYCARELGQNFD |
| | YWGQGTTVTVSS (SEQ ID NO: 28) |

In some embodiments, provided herein are antibodies or antigen-binding fragments thereof that specifically bind ILT7 comprising a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 9. In some embodiments, provided herein are antibodies or antigen-binding fragments thereof that specifically bind ILT7 comprising a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 10.

In some embodiments, provided herein are antibodies or antigen-binding fragments thereof that specifically bind ILT7 comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 9; and (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 10. In some embodiments, provided herein are antibodies or antigen-binding fragments thereof that specifically bind ILT7 comprising a VL and a VH, wherein the VL and VH have the amino acid sequences of SEQ ID NOs: 9 and 10, respectively.

In some embodiments, provided herein are antibodies or antigen-binding fragments thereof that specifically bind ILT7 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 9. In some embodiments, the anti-ILT7 antibody or antigen-binding fragment thereof has a VL having at least 85% sequence identity to SEQ ID NO: 9. In some embodiments, the anti-ILT7 antibody or antigen-binding fragment thereof has a VL having at least 90% sequence identity to SEQ ID NO: 9. In some embodiments, the anti-ILT7 antibody or antigen-binding fragment thereof has a VL having at least 95% sequence identity to SEQ ID NO: 9. In some embodiments, the anti-ILT7 antibody or antigen-binding fragment thereof has a VL having at least 98% sequence identity to SEQ ID NO: 9. In some embodiments, provided herein are antibodies or antigen-binding fragments thereof that specifically bind ILT7 comprising a VL having the amino acid sequence of SEQ ID NO: 9.

In some embodiments, provided herein are antibodies or antigen-binding fragments thereof that specifically bind ILT7 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 10. In some embodiments, the anti-ILT7 antibody or antigen-binding fragment thereof has a VH having at least 85% sequence identity to SEQ ID NO: 10. In some embodiments, the anti-ILT7 antibody or antigen-binding fragment thereof has a VH having at least 90% sequence identity to SEQ ID NO: 10. In some embodiments, the anti-ILT7 antibody or antigen-binding fragment thereof has a VH having at least 95% sequence identity to SEQ ID NO: 10. In some embodiments, the anti-ILT7 antibody or antigen-binding fragment thereof has a VH having at least 98% sequence identity to SEQ ID NO: 10. In some embodiments, provided herein are antibodies or antigen-binding fragments thereof that specifically bind ILT7 comprising a VH having the amino acid sequence of SEQ ID NO: 10.

In some embodiments, an anti-ILT7 antibody or antigen-binding fragment thereof comprises a humanized antibody or antigen-binding fragment. In some embodiments, an anti-ILT7 antibody or antigen-binding fragment thereof comprises a VL CDR1, VL CDR2, VL CDR3, VH CDR1, VH CDR2, and/or VH CDR3 from an antibody or antigen-binding fragment described herein. In some embodiments, an anti-ILT7 antibody or antigen-binding fragment thereof comprises a variant of an anti-ILT7 antibody or antigen-binding fragment described herein. A variant of an anti-ILT7 antibody or antigen-binding fragment can comprise one to 30 amino acid substitutions, additions, and/or deletions in the anti-ILT7 antibody or antigen-binding fragment. A variant of an anti-ILT7 antibody or antigen-binding fragment can comprise one to 25 amino acid substitutions, additions, and/or deletions in the anti-ILT7 antibody or antigen-binding fragment. In some embodiments, a variant of an anti-ILT7 antibody or antigen-binding fragment comprises one to 20 substitutions, additions, and/or deletions in the anti-ILT7 antibody or antigen-binding fragment. In some embodiments, a variant of an anti-ILT7 antibody or antigen-binding fragment comprises one to 15 substitutions, additions, and/or deletions in the anti-ILT7 antibody or antigen-binding fragment. In some embodiments, a variant of an anti-ILT7 antibody or antigen-binding fragment comprises one to 10 substitutions, additions, and/or deletions in the anti-ILT7 antibody or antigen-binding fragment. In some embodiments, a variant of an anti-ILT7 antibody or antigen-binding fragment comprises one to five conservative amino acid substitutions, additions, and/or deletions in the anti-ILT7 antibody or antigen-binding fragment. In some embodiments, a variant of an anti-ILT7 antibody or antigen-binding fragment comprises one to three amino acid substitutions, additions, and/or deletions in the anti-ILT7 antibody or antigen-binding fragment. In some embodiments, the amino acid substitutions, additions, and/or deletions are conservative amino acid substitutions. In some embodiments, the conservative amino acid substitution(s) is in a CDR of the antibody or antigen-binding fragment. In some embodiments, the conservative amino acid substitution(s) is not in a CDR of the antibody or antigen-binding fragment. In some embodiments, the conservative amino acid substitution(s) is in a framework region of the antibody or antigen-binding fragment.

In some embodiments, provided herein are humanized antibodies or antigen-binding fragments thereof that specifically bind ILT7 comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs: 19-22; and/or (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs: 23-28. In some embodiments, the humanized antibodies or antigen-binding fragments thereof comprise a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 19; and a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 26.

In some embodiments, provided herein are humanized antibodies or antigen-binding fragments thereof that specifically bind ILT7 comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs: 19-22, the VL also having, as defined by Kabat or Chothia, VL CDR1, VL CDR2, and VL CDR3 having the amino acid sequences of SEQ ID NOs: 11, 12, and 13, respectively; and/or (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs: 23-28, the VH also having (1) as defined by Kabat, VH CDR1, VH CDR2, and VH CDR3 having the amino acid sequences of SEQ ID NOs: 14, 15, and 16, respectively, or (2) as defined by Chothia, VH CDR1, VH CDR2, and VH CDR3 having the amino acid sequences of SEQ ID NOs: 17, 18, and 16, respectively. In some embodiments. VL has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 19, the VL also having, as defined by Kabat or Chothia, VL CDR1, VL CDR2, and VL CDR3 having the amino acid sequences of SEQ ID NOs: 11, 12, and 13, respectively; and VH has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NOs: 26, the VH also having (1) as defined by Kabat, VH CDR1, VH CDR2, and VH CDR3 having the amino acid sequences of SEQ ID NOs: 14, 15, and 16, respectively, or (2) as defined by Chothia, VH CDR1, VH CDR2, and VH CDR3 having the amino acid sequences of SEQ ID NOs: 17, 18, and 16, respectively.

In some embodiments, provided herein are antibodies or antigen-binding fragments thereof that specifically bind ILT7 comprising a VL and a VH, wherein the VL and VH have the amino acid sequences of SEQ ID NOs: 19 and 23, respectively. In some embodiments, VL and VH have the amino acid sequences of SEQ ID NOs: 19 and 24, respectively. In some embodiments, VL and VH have the amino acid sequences of SEQ ID NOs: 19 and 25, respectively. In some embodiments, VL and VH have the amino acid sequences of SEQ ID NOs: 19 and 26, respectively. In some embodiments, VL and VH have the amino acid sequences of SEQ ID NOs: 19 and 27, respectively. In some embodiments, VL and VH have the amino acid sequences of SEQ ID NOs: 19 and 28, respectively. In some embodiments, provided herein are antibodies or antigen-binding fragments thereof that specifically bind ILT7 comprising a VL and a VH, wherein the VL and VH have the amino acid sequences of SEQ ID NOs: 20 and 23, respectively. In some embodiments, VL and VH have the amino acid sequences of SEQ ID NOs: 20 and 24, respectively. In some embodiments, VL and VH have the amino acid sequences of SEQ ID NOs: 20 and 25, respectively. In some embodiments, VL and VH have the amino acid sequences of SEQ ID NOs: 20 and 26, respectively. In some embodiments, VL and VH have the amino acid sequences of SEQ ID NOs: 20 and 27, respectively. In some embodiments, VL and VH have the amino acid sequences of SEQ ID NOs: 20 and 28, respectively. In some embodiments, provided herein are antibodies or antigen-binding fragments thereof that specifically bind ILT7 comprising a VL and a VH, wherein the VL and VH have the amino acid sequences of SEQ ID NOs: 21 and 23, respectively. In some embodiments, VL and VH have the amino acid sequences of SEQ ID NOs: 21 and 24, respectively. In some embodiments, VL and VH have the amino acid sequences of SEQ ID NOs: 21 and 25, respectively. In some embodiments, VL and VH have the amino acid sequences of SEQ ID NOs: 21 and 26, respectively. In some embodiments, VL and VH have the amino acid sequences of SEQ ID NOs: 21 and 27, respectively. In some embodiments, VL and VH have the amino acid sequences of SEQ ID NOs: 21 and 28, respectively. In some embodiments, provided herein are antibodies or antigen-binding fragments thereof that specifically bind ILT7 comprising a VL and a VH, wherein the VL and VH have the amino acid sequences of SEQ ID NOs: 22 and 23, respectively. In some embodiments, VL and VH have the amino acid sequences of SEQ ID NOs: 22 and 24, respectively. In some embodiments, VL and VH have the amino acid sequences of SEQ ID NOs: 22 and 25, respectively. In some embodiments, VL and VH have the amino acid sequences of SEQ ID NOs: 22 and 26, respectively. In some embodiments, VL and VH have the amino acid sequences of SEQ ID NOs: 22 and 27, respectively. In some embodiments, VL and VH have the amino acid sequences of SEQ ID NOs: 22 and 28, respectively.

In some embodiments, provided herein are humanized antibodies or antigen-binding fragments thereof that specifically bind ILT7 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 19. The humanized anti-ILT7 antibody or antigen-binding fragment thereof can have a VL having at least 85% sequence identity to SEQ ID NO: 19. The humanized anti-ILT7 antibody or antigen-binding fragment thereof can have a VL having at least 90% sequence identity to SEQ ID NO: 19. The humanized anti-ILT7 antibody or antigen-binding fragment thereof can have a VL having at least 95% sequence identity to SEQ ID NO: 19. The humanized anti-ILT7 antibody or antigen-binding fragment thereof can have a VL having at least 98% sequence identity to SEQ ID NO: 19. In some embodiments, provided herein are humanized antibodies or antigen-binding fragments thereof that specifically bind ILT7 comprising a VL having the amino acid sequence of SEQ ID NO: 19.

In some embodiments, provided herein are humanized antibodies or antigen-binding fragments thereof that specifically bind ILT7 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 20. The humanized anti-ILT7 antibody or antigen-binding fragment thereof can have a VL having at least 85% sequence identity to SEQ ID NO: 20. The humanized anti-ILT7 antibody or antigen-binding fragment thereof can have a VL having at least 90% sequence identity to SEQ ID NO: 20. The humanized anti-ILT7 antibody or antigen-binding fragment thereof can have a VL having at least 95% sequence identity to SEQ ID NO: 20. The humanized anti-ILT7 antibody or antigen-binding fragment thereof can have a VL having at least 98% sequence identity to SEQ ID NO: 20. In some embodiments, provided herein are humanized antibodies or antigen-binding fragments thereof that specifically bind ILT7 comprising a VL having the amino acid sequence of SEQ ID NO: 20.

In some embodiments, provided herein are humanized antibodies or antigen-binding fragments thereof that specifically bind ILT7 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 21. The humanized anti-ILT7 antibody or antigen-binding fragment thereof can have a VL having at least 85% sequence identity to SEQ ID NO: 21. The humanized anti-ILT7 antibody or antigen-binding fragment thereof can have a VL having at least 90% sequence identity to SEQ ID NO: 21. The humanized anti-ILT7 antibody or antigen-binding fragment thereof can have a VL having at least 95% sequence identity to SEQ ID NO: 21. The humanized anti-ILT7 antibody or antigen-binding fragment thereof can have a VL having at least 98% sequence identity to SEQ ID NO: 21. In some embodiments, provided herein are humanized antibodies or antigen-binding fragments thereof that specifically bind ILT7 comprising a VL having the amino acid sequence of SEQ ID NO: 21.

In some embodiments, provided herein are humanized antibodies or antigen-binding fragments thereof that specifically bind ILT7 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 22. The humanized anti-ILT7 antibody or antigen-binding fragment thereof can have a VL having at least 85% sequence identity to SEQ ID NO: 22. The humanized anti-ILT7 antibody or antigen-binding fragment thereof can have a VL having at least 90% sequence identity to SEQ ID NO: 22. The humanized anti-ILT7 antibody or antigen-binding fragment thereof can have a VL having at least 95% sequence identity to SEQ ID NO: 22. The humanized anti-ILT7 antibody or antigen-binding fragment thereof can have a VL having at least 98% sequence identity to SEQ ID NO: 22. In some embodiments, provided herein are humanized antibodies or antigen-binding fragments thereof that specifically bind ILT7 comprising a VL having the amino acid sequence of SEQ ID NO: 22.

In some embodiments, provided herein are humanized antibodies or antigen-binding fragments thereof that specifically bind ILT7 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 23. The humanized anti-ILT7 antibody or antigen-binding fragment thereof can have a VH having at least 85% sequence identity to SEQ ID NO: 23. The humanized anti-ILT7 antibody or antigen-binding fragment thereof can have a VH having at least 90% sequence identity to SEQ ID NO: 23. The humanized anti-ILT7 antibody or antigen-binding fragment thereof can have a VH having at least 95% sequence identity to SEQ ID NO: 23. The humanized anti-ILT7 antibody or antigen-binding fragment thereof can have a VH having at least 98% sequence identity to SEQ ID NO: 23. In some embodiments, provided herein are humanized antibodies or antigen-binding fragments thereof that specifically bind ILT7 comprising a VH having the amino acid sequence of SEQ ID NO: 23.

In some embodiments, provided herein are humanized antibodies or antigen-binding fragments thereof that specifically bind ILT7 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 24. The humanized anti-ILT7 antibody or antigen-binding fragment thereof can have a VH having at least 85% sequence identity to SEQ ID NO: 24. The humanized anti-ILT7 antibody or antigen-binding fragment thereof can have a VH having at least 90% sequence identity to SEQ ID NO: 24. The humanized anti-ILT7 antibody or antigen-binding fragment thereof can have a VH having at least 95% sequence identity to SEQ ID NO: 24. The humanized anti-ILT7 antibody or antigen-binding fragment thereof can have a VH having at least 98% sequence identity to SEQ ID NO: 24. In some embodiments, provided herein are humanized antibodies or antigen-binding fragments thereof that specifically bind ILT7 comprising a VH having the amino acid sequence of SEQ ID NO: 24.

In some embodiments, provided herein are humanized antibodies or antigen-binding fragments thereof that specifically bind ILT7 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 25. The humanized anti-ILT7 antibody or antigen-binding fragment thereof can have a VH having at least 85% sequence identity to SEQ ID NO: 25. The humanized anti-ILT7 antibody or antigen-binding fragment thereof can have a VH having at least 90% sequence identity to SEQ ID NO: 25. The humanized anti-ILT7 antibody or antigen-binding fragment thereof can have a VH having at least 95% sequence identity to SEQ ID NO: 25. The humanized anti-ILT7 antibody or antigen-binding fragment thereof can have a VH having at least 98% sequence identity to SEQ ID NO: 25. In some embodiments, provided herein are humanized antibodies or antigen-binding fragments thereof that specifically bind ILT7 comprising a VH having the amino acid sequence of SEQ ID NO: 25.

In some embodiments, provided herein are humanized antibodies or antigen-binding fragments thereof that specifically bind ILT7 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 26. The humanized anti-ILT7 antibody or antigen-binding fragment thereof can have a VH having at least 85% sequence identity to SEQ ID NO: 26. The humanized anti-ILT7 antibody or antigen-binding fragment thereof can have a VH having at least 90% sequence identity to SEQ ID NO: 26. The humanized anti-ILT7 antibody or antigen-binding fragment thereof can have a VH having at least 95% sequence identity to SEQ ID NO: 26. The humanized anti-ILT7 antibody or antigen-binding fragment thereof can have a VH having at least 98% sequence identity to SEQ ID NO: 26. In some embodiments, provided herein are humanized antibodies or antigen-binding fragments thereof that specifically bind ILT7 comprising a VH having the amino acid sequence of SEQ ID NO: 26.

In some embodiments, provided herein are humanized antibodies or antigen-binding fragments thereof that specifically bind ILT7 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 27. The humanized anti-ILT7 antibody or antigen-binding fragment thereof can have a VH having at least 85% sequence identity to SEQ ID NO: 27. The humanized anti-ILT7 antibody or antigen-binding fragment thereof can have a VH having at least 90% sequence identity to SEQ ID NO: 27. The humanized anti-ILT7 antibody or antigen-binding fragment thereof can have a VH having at least 95% sequence identity to SEQ ID NO: 27. The humanized anti-ILT7 antibody or antigen-binding fragment thereof can have a VH having at least 98% sequence identity to SEQ ID NO: 27. In some embodiments, provided herein are humanized antibodies or antigen-binding fragments thereof that specifically bind ILT7 comprising a VH having the amino acid sequence of SEQ ID NO: 27.

In some embodiments, provided herein are humanized antibodies or antigen-binding fragments thereof that specifically bind ILT7 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 28. The humanized anti-ILT7 antibody or antigen-binding fragment thereof can have a VH having at least 85% sequence identity to SEQ ID NO: 28. The humanized anti-ILT7 antibody or antigen-binding fragment thereof can have a VH having at least 90% sequence identity to SEQ ID NO: 28. The humanized anti-ILT7 antibody or antigen-binding fragment thereof can have a VH having at least 95% sequence identity to SEQ ID NO: 28. The humanized anti-ILT7 antibody or antigen-binding fragment thereof can have a VH having at least 98% sequence identity to SEQ ID NO: 28. In some embodiments, provided herein are humanized antibodies or antigen-binding fragments thereof that specifically bind ILT7 comprising a VH having the amino acid sequence of SEQ ID NO: 28.

The anti-ILT7 antibodies or antigen-binding fragments thereof can comprise a combination of any VL disclosed herein and any VH disclosed herein. In some embodiments, the VL and VH are connected by a linker. The linker can be a flexible linker or a rigid linker. In some embodiments, the linker has the amino acid sequence of (GGGGS)n, n=1, 2, 3, 4, or 5 (SEQ ID NO: 35). In some embodiments, the linker has the amino acid sequence of (EAAAK)n, n=1, 2, 3, 4, or 5 (SEQ ID NO: 36). In some embodiments, the linker has the amino acid sequence of (PA)nP, n=1, 2, 3, 4, or 5 (SEQ ID NO: 37).

In some embodiments, provided herein are anti-ILT7 antibodies or antigen-binding fragments thereof that comprise VL CDRs from a VL described herein (SEQ ID NO: 9, 19, 20, 21, or 22), and/or VH CDRs from a VH described herein (SEQ ID NO: 10, 23, 24, 25, 26, 27 or 28). Methods to identify CDRs are well known in the art. For example, software programs (abYsis) on publicly available website are known to those of skill in the art for analysis of antibody sequence and determination of CDRs.

In some embodiments, provided herein are antibodies or antigen-binding fragments thereof that specifically bind ILT7 comprising (a) a VL comprising VL CDRs 1, 2, and 3 from a VL having the amino acid sequence of SEQ ID NO: 9; and/or (b) a VH comprising VH CDRs 1, 2, and 3 from a VH having an amino acid sequence of SEQ ID NO: 10.

In some embodiments, provided herein are antibodies or antigen-binding fragments thereof that specifically bind ILT7 comprising (a) a VL comprising VL CDRs 1, 2, and 3 from a VL having an amino acid sequence selected from the group consisting of SEQ ID NOs: 19-22; and/or (b) a VH comprising VH CDRs 1, 2, and 3 from a VH having an amino acid sequence selected from the group consisting of SEQ ID NOs: 23-28.

In some embodiments, provided herein are antibodies or antigen-binding fragments thereof that specifically bind ILT7 comprising a VL, wherein the VL comprises VL CDRs 1, 2, and 3 from a VL having the amino acid sequence of SEQ ID NO: 9. In some embodiments, provided herein are antibodies or antigen-binding fragments thereof that specifically bind ILT7 comprising a VL, wherein the VL comprises VL CDRs 1, 2, and 3 from a VL having the amino acid sequence of SEQ ID NO: 19. In some embodiments, provided herein are antibodies or antigen-binding fragments thereof that specifically bind ILT7 comprising a VL, wherein the VL comprises VL CDRs 1, 2, and 3 from a VL having the amino acid sequence of SEQ ID NO: 20. In some embodiments, provided herein are antibodies or antigen-binding fragments thereof that specifically bind ILT7 comprising a VL, wherein the VL comprises VL CDRs 1, 2, and 3 from a VL having the amino acid sequence of SEQ ID NO: 21. In some embodiments, provided herein are antibodies or antigen-binding fragments thereof that specifically bind ILT7 comprising a VL, wherein the VL comprises VL CDRs 1, 2, and 3 from a VL having the amino acid sequence of SEQ ID NO: 22.

In some embodiments, provided herein are humanized antibodies or antigen-binding fragments thereof that specifically bind ILT7 comprising a VH, wherein the VH comprises VH CDRs 1, 2, and 3 from a VH having the amino acid sequence of SEQ ID NO: 10. In some embodiments, provided herein are antibodies or antigen-binding fragments thereof that specifically bind ILT7 comprising a VH, wherein the VH comprises VH CDRs 1, 2, and 3 from a VH having the amino acid sequence of SEQ ID NO: 23. In some embodiments, provided herein are antibodies or antigen-binding fragments thereof that specifically bind ILT7 comprising a VH, wherein the VH comprises VH CDRs 1, 2, and 3 from a VH having the amino acid sequence of SEQ ID NO: 24. In some embodiments, provided herein are antibodies or antigen-binding fragments thereof that specifically bind ILT7 comprising a VH, wherein the VH comprises VH CDRs 1, 2, and 3 from a VH having the amino acid sequence of SEQ ID NO: 25. In some embodiments, provided herein are antibodies or antigen-binding fragments thereof that specifically bind ILT7 comprising a VH, wherein the VH comprises VH CDRs 1, 2, and 3 from a VH having the amino acid sequence of SEQ ID NO: 26. In some embodiments, provided herein are antibodies or antigen-binding fragments thereof that specifically bind ILT7 comprising a VH, wherein the VH comprises VH CDRs 1, 2, and 3 from a VH having the amino acid sequence of SEQ ID NO: 27. In some embodiments, provided herein are antibodies or antigen-binding fragments thereof that specifically bind ILT7 comprising a VH, wherein the VH comprises VH CDRs 1, 2, and 3 from a VH having the amino acid sequence of SEQ ID NO: 28.

In some embodiments, the anti-ILT7 antibody or antigen-binding fragment thereof provided herein is the antibody designated as cmAb12 (chimeric Ab12). In some embodiments, the anti-ILT7 antibody or antigen-binding fragment thereof provided herein has a VL from cmAb12 (SEQ ID NO: 9). In some embodiments, the anti-ILT7 antibody or antigen-binding fragment thereof provided herein has a VH from cmAb12 (SEQ ID NO: 10). The anti-ILT7 antibody or antigen-binding fragment thereof provided herein can have both the VL and the VH from cmAb12. In some embodiments, the anti-ILT7 antibody or antigen-binding fragment thereof provided herein has a VL that comprises VL CDRs 1, 2, and 3 from the VL from cmAb12 (SEQ ID NO: 9). In some embodiments, the anti-ILT7 antibody or antigen-binding fragment thereof provided herein has a VH that comprises VH CDRs 1, 2, and 3 from the VH from cmAb12 (SEQ ID NO: 10). The anti-ILT7 antibody or antigen-binding fragment thereof provided herein can have a VL comprising VL CDRs 1, 2, and 3 and a VH comprising VH CDRs 1, 2, and 3 from the VL and VH of cmAb12, respectively. In some embodiments, the anti-ILT7 antibody or antigen-binding fragment thereof provided herein is a variant of emAb12. The cmAb12 variant can have a VL that is a variant of the VL of cmAb12 having up to about 3, about 5, about 8, about 10, about 12, or about 15 amino acid substitutions, additions, and/or deletions in SEQ ID NO: 9. The cmAb12 variant can have a VL that is a variant of the VL of cmAb12 having up to about 5 amino acid substitutions, additions, and/or deletions in SEQ ID NO: 9. The cmAb12 variant can have a VH that is a variant of the VH of cmAb12 having up to about 3, about 5, about 8, about 10, about 12, or about 15 amino acid substitutions, additions, and/or deletions in SEQ ID NO: 10. The cmAb12 variant can have a VH that is a variant of the VH of cmAb12 having up to about 5 amino acid substitutions, additions, and/or deletions in SEQ ID NO: 10. The amino acid substitutions, additions, and/or deletions can be in the VH CDRs or VL CDRs. In some embodiments, the amino acid substitutions, additions, and/or deletions are not in the CDRs. In some embodiments, the variant of cmAb12 has up to about 5 conservative amino acid substitutions. In some embodiments, the variant of cmAb12 has up to 3 conservative amino acid substitutions. In some embodiments, the anti-ILT7 antibody or antigen-binding fragment thereof provided herein is a humanized antibody or antigen-binding fragment derived from cmAb12. In some embodiments, the anti-ILT7 antibody or antigen-binding fragment thereof provided herein is a human antibody or antigen-binding fragment derived from cmAb12.

In some embodiments, provided herein are humanized antibodies of Ab12 (e.g., humanized Ab12, hu-cmAb12, hu-Ab12, or hu-12). In some embodiments, the humanized anti-ILT7 antibody or antigen-binding fragment thereof provided herein comprises a VL having an amino acid sequence selected from the group consisting of SEQ ID NOs: 19-22. In some embodiments, the humanized anti-ILT7 antibody or antigen-binding fragment thereof provided herein comprises a VH having an amino acid sequence selected from SEQ ID NOs: 23-28. In some embodiments, the humanized anti-ILT7 antibody or antigen-binding fragment thereof provided herein comprises a VL having an amino acid sequence selected from the group consisting of SEQ ID NOs: 19-22 and a VH having an amino acid sequence selected from the group consisting of SEQ ID NOs: 23-28. In some embodiments, the anti-ILT7 antibody or antigen-binding fragment thereof provided herein is a variant of a humanized Ab12 provided herein. The variant can have a VL that is a variant of the VL of a humanized Ab12 having up to about 3, about 5, about 8, about 10, about 12, or about 15 amino acid substitutions, additions, and/or deletions in an amino acid sequence selected from the group consisting of SEQ ID NOs: 19-22. The variant can have a VL that is a variant of the VL of a humanized Ab12 having up to about 5 amino acid substitutions, additions, and/or deletions in an amino acid sequence selected from the group consisting of SEQ ID NOs: 19-22. The variant can have a VH that is a variant of the VH of a humanized Ab12 having up to about 3, about 5, about 8, about 10, about 12, or about 15 amino acid substitutions, additions, and/or deletions in an amino acid sequence selected from the group consisting of SEQ ID NOs: 23-28. The variant can have a VH that is a variant of the VH of a humanized Ab12 having up to about 5 amino acid substitutions, additions, and/or deletions in an amino acid sequence selected from the group consisting of SEQ ID NOs: 23-28. In some embodiments, the variant of a humanized Ab12 has up to about 5 conservative amino acid substitutions. In some embodiments, the humanized antibody is the antibody “hu-cmAb12,” i.e., a humanized antibody comprising a VL having the amino acid sequence of SEQ ID NO: 19, and a VH having the amino acid sequence of SEQ ID NO: 26. Selected properties of humanized antibody hu-cmAB12 are demonstrated in Examples 8-17.

In some embodiments, anti-ILT7 antibodies provided herein are IgA, IgD, IgE, IgG, or IgM antibodies. In some embodiments, the antibody is an IgA antibody. In some embodiments, the antibody is an IgD antibody. In some embodiments, the antibody is an IgE antibody. In some embodiments, the antibody is an IgG antibody. In some embodiments, the antibody is an IgM antibody. In some embodiments, the antibodies provided herein can be an IgG1 antibody, an IgG2 antibody, an IgG3 antibody, or an IgG4 antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody.

In some embodiments, anti-ILT7 antibodies provided herein comprise a light chain and a heavy chain. The light chain can comprise a light chain constant domain (CL) and a light chain variable domain (VL). The heavy chain can comprise a heavy chain variable domain (VH) and a heavy chain constant domain (CH). The VL/VH can be any VL/VH disclosed herein. In some embodiments, the light chain constant region (CL) is kappa CL (CK; SEQ ID NO: 29). In some embodiments, the light chain constant region (CL) is lambda CL (CC; SEQ ID NO: 30). In some embodiments, the heavy chain comprises a heavy chain constant domain (CH) from human IgA. In some embodiments, the heavy chain comprises a heavy chain constant domain (CH) from human IgD. In some embodiments, the heavy chain comprises a heavy chain constant domain (CH) from human IgE. In some embodiments, the heavy chain comprises a heavy chain constant domain (CH) from human IgG. In some embodiments, the heavy chain comprises a heavy chain constant domain (CH) from human IgM. In some embodiments, the heavy chain comprises a heavy chain constant domain (CH) from human IgG1 (e.g., SEQ ID NO: 31). In some embodiments, the heavy chain comprises a heavy chain constant domain (CH) from human IgG2 (e.g., SEQ ID NO: 32). In some embodiments, the heavy chain comprises a heavy chain constant domain (CH) from human IgG3 (e.g., SEQ ID NO: 33). In some embodiments, the heavy chain comprises a heavy chain constant domain (CH) from human IgG4 (e.g., SEQ ID NO: 34). Expressly contemplated here are any and all combinations of the VL/VH pairs disclosed herein that specifically bind ILT7 (e.g., human ILT7) and the CL/CH disclosed herein or otherwise known in the art.

| Light Chain | |
|---|---|
| Constant Region | Sequences |
| kappa CL (Cκ) | RTVAAPSVFIFPPSDEQLKSGTASVVCLL |
| | NNFYPREAKVQWKVDNALQSGNSQESVTE |
| | QDSKDSTYSLSSTLTLSKADYEKHKVYAC |
| | EVTHQGLSSPVTKSFNRGEC |
| | (SEQ ID NO: 29) |
| lambda CL (Cλ) | GQPKAAPSVTLFPPSSEELQANKATLVCL |
| | ISDFYPGAVTVAWKADSSPVKAGVETTTP |
| | SKQSNNKYAASSYLSLTPEQWKSHRSYSC |
| | QVTHEGSTVEKTVAPTECS |
| | (SEQ ID NO: 30) |

| Heavy Chain | |
|---|---|
| Constant Region | Sequences |
| IgG1 CH | ASTKGPSVFPLAPSSKSTSGGTAALGCLVKDYFPEPVTVSWNSG |
| | ALTSGVHTFPAVLQSSGLYSLSSVVTVPSSSLGTQTYICNVNHK |
| | PSNTKVDKKVEPKSCDKTHTCPPCPAPELLGGPSVFLFPPKPKD |
| | TLMISRTPEVTCVVVDVSHEDPEVKFNWYVDGVEVHNAKTKP |
| | REEQYNSTYRVVSVLTVLHQDWLNGKEYKCKVSNKALPAPIE |
| | KTISKAKGQPREPQVYTLPPSRDELTKNQVSLTCLVKGFYPSDI |
| | AVEWESNGQPENNYKTTPPVLDSDGSFFLYSKLTVDKSRWQQ |
| | GNVFSCSVMHEALHNHYTQKSLSLSPGK (SEQ ID NO: 31) |
| IgG2 CH | ASTKGPSVFPLAPCSRSTSESTAALGCLVKDYFPEPVTVSWNSG |
| | ALTSGVHTFPAVLQSSGLYSLSSVVTVPSSNFGTQTYTCNVDHK |
| | PSNTKVDKTVERKCCVECPPCPAPPVAGPSVFLFPPKPKDTLMI |
| | SRTPEVTCVVVDVSHEDPEVQFNWYVDGVEVHNAKTKPREEQ |
| | FNSTFRVVSVLTVVHQDWLNGKEYKCKVSNKGLPAPIEKTISK |
| | TKGQPREPQVYTLPPSREEMTKNQVSLTCLVKGFYPSDISVEWE |
| | SNGQPENNYKTTPPMLDSDGSFFLYSKLTVDKSRWQQGNVFSC |
| | SVMHEALHNHYTQKSLSLSPGK (SEQ ID NO: 32) |
| IgG3 CH | ASTKGPSVFPLAPCSRSTSGGTAALGCLVKDYFPEPVTVSWNSG |
| | ALTSGVHTFPAVLQSSGLYSLSSVVTVPSSSLGTQTYTCNVNHK |
| | PSNTKVDKRVELKTPLGDTTHTCPRCPEPKSCDTPPPCPRCPEP |
| | KSCDTPPPCPRCPEPKSCDTPPPCPRCPAPELLGGPSVFLFPPKPK |
| | DTLMISRTPEVTCVVVDVSHEDPEVQFKWYVDGVEVHNAKTK |
| | PREEQYNSTFRVVSVLTVLHQDWLNGKEYKCKVSNKALPAPIE |
| | KTISKTKGQPREPQVYTLPPSREEMTKNQVSLTCLVKGFYPSDI |
| | AVEWESSGQPENNYNTTPPMLDSDGSFFLYSKLTVDKSRWQQ |
| | GNIFSCSVMHEALHNRFTQKSLSLSPGK (SEQ ID NO: 33) |
| IgG4 CH | ASTKGPSVFPLAPCSRSTSESTAALGCLVKDYFPEPVTVSWNSG |
| | ALTSGVHTFPAVLQSSGLYSLSSVVTVPSSSLGTKTYTCNVDHK |
| | PSNTKVDKRVESKYGPPCPSCPAPEFLGGPSVFLFPPKPKDTLMI |
| | SRTPEVTCVVVDVSQEDPEVQFNWYVDGVEVHNAKTKPREEQ |
| | FNSTYRVVSVLTVLHQDWLNGKEYKCKVSNKGLPSSIEKTISK |
| | AKGQPREPQVYTLPPSQEEMTKNQVSLTCLVKGFYPSDIAVEW |
| | ESNGQPENNYKTTPPVLDSDGSFFLYSRLTVDKSRWQEGNVFS |
| | CSVMHEALHNHYTQKSLSLSLGK (SEQ ID NO: 34) |

In some embodiments, the antibodies provided herein have a light chain constant region (CL) having at least 85%, at least 90%, at least 95%, at least 98%, or at least 99% sequence identity to SEQ ID NO: 29. In some embodiments, the antibodies provided herein have a CL having the amino acid sequence of SEQ ID NO: 29. In some embodiments, the antibodies provided herein have a light chain constant region (CL) having at least 85%, at least 90%, at least 95%, at least 98%, or at least 99% sequence identity to SEQ ID NO: 30. In some embodiments, the antibodies provided herein have a CL having the amino acid sequence of SEQ ID NO: 30. In some embodiments, the antibodies provided herein have a heavy chain constant region (CH) having at least 85%, at least 90%, at least 95%, at least 98%, or at least 99% sequence identity to SEQ ID NO: 31. In some embodiments, the antibodies provided herein have a CH having the amino acid sequence of SEQ ID NO: 31. In some embodiments, the antibodies provided herein have a CH having at least 85%, at least 90%, at least 95%, at least 98%, or at least 99% sequence identity to SEQ ID NO: 32. In some embodiments, the antibodies provided herein have a CH having the amino acid sequence of SEQ ID NO: 32. In some embodiments, the antibodies provided herein have a CH having at least 85%, at least 90%, at least 95%, at least 98%, or at least 99% sequence identity to SEQ ID NO: 33. In some embodiments, the antibodies provided herein have a CH having the amino acid sequence of SEQ ID NO: 33. In some embodiments, the antibodies provided herein have a CH having at least 85%, at least 90%, at least 95%, at least 98%, or at least 99% sequence identity to SEQ ID NO: 34. In some embodiments, the antibodies provided herein have a CH having the amino acid sequence of SEQ ID NO: 34.

In some embodiments, provided herein are also antibodies or antigen-binding fragments that compete with the antibody or antigen-binding fragment provided above for binding to ILT7 (e.g., human ILT7). Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments, e.g., BIACORE© surface plasmon resonance (SPR) analysis. In some embodiments, an anti-ILT7 antibody or antigen-binding fragment competes with, and inhibits binding of another antibody or antigen-binding fragment to ILT7 by at least 50%, 60%, 70%, 80%, 90% or 100%. Competition assays can be conducted as described, for example, in Ed Harlow and David Lane, Cold Spring Harb Protoc; 2006; doi: 10.H01/pdb.prot4277 or in Chapter 11 of “Using Antibodies” by Ed Harlow and David Lane, Cold Spring Harbor Laboratory Press, Cold Spring Harbor, NY, USA 1999.

In some embodiments, provided herein are antibodies or antigen-binding fragments that compete with an anti-ILT7 antibody or antigen-binding fragment disclosed herein for binding to ILT7 (e.g., human ILT7). In some embodiments, provided herein are antibodies or antigen-binding fragments that compete with chimeric Ab12 for binding to ILT7 (e.g., human ILT7). In some embodiments, provided herein are antibodies or antigen-binding fragments that compete with a humanized Ab12 disclosed herein for binding to ILT7 (e.g., human ILT7).

Epitope mapping is a method of identifying the binding site, region, or epitope on a target protein where an antibody binds. A variety of methods are known in the art for mapping epitopes on target proteins. These methods include mutagenesis, including but not limited to, shotgun mutagenesis, site-directed mutagenesis, and alanine scanning; domain or fragment scanning; peptide scanning (e.g., Pepscan technology); display methods (e.g., phage display, microbial display, and ribosome/mRNA display); methods involving proteolysis and mass spectroscopy; and structural determination (e.g., X-ray crystallography and NMR). In some embodiments, anti-ILT7 antibodies or antigen-binding fragments described herein are characterized by assays including, but not limited to, N-terminal sequencing, amino acid analysis, HPLC, mass spectrometry, ion exchange chromatography, and papain digestion.

As provided in further detail in Experimental section below, Ab12 does not compete for binding to human ILT7 with benchmark antibody daxdilimab. In some embodiments, the anti-ILT7 antibodies and antigen-binding fragments provided herein do not compete for human ILT7 binding with daxdilimab.

The anti-ILT7 antibodies or antigen-binding fragments of the present disclosure can be analyzed for their physical, chemical, and/or biological properties by various methods known in the art. In some embodiments, an anti-ILT7 antibody is tested for its ability to bind ILT7 (e.g., human ILT7). In some embodiments, an anti-ILT7 antibody is tested for its ability to bind an FcγR. In some embodiments, an anti-ILT7 antibody is tested for its ability to bind FcγRIIA/CD32A. In some embodiments, an anti-ILT7 antibody is tested for its ability to bind FcγRIIIA/CD16A. Binding assays include, but are not limited to, BLI, SPR (e.g., Biacore), ELISA, and FACS. In addition, antibodies can be evaluated for solubility, stability, thermostability, viscosity, expression levels, expression quality, and/or purification efficiency.

In some embodiments, anti-ILT7 antibodies or antigen-binding fragments described herein bind to human ILT7 with high affinity, for example, with a K_{D} of 10⁻⁶ M or less, 5×10⁻⁷ M or less, 10⁻⁷ M or less, 5×10⁻⁸ M or less, 10⁻⁸M or less, 5×10⁻⁹ M or less, 10⁻⁹ M or less, 5×10⁻¹⁰ M or less, or 10⁻¹⁰ M or less. In some embodiments, anti-ILT7 antibodies or antigen-binding fragments described herein bind to human ILT7 with a K_{D} of 5×10⁻⁷ M or less. In some embodiments, anti-ILT7 antibodies or antigen-binding fragments described herein bind to human ILT7 with high affinity, for example, with a K_{D} of about 10⁻⁶ M, about 5×10⁻⁷ M, about 10⁻⁷ M, about 5×10⁻⁸ M, about 10⁻⁸ M, about 5×10⁻⁹ M, about 10⁻⁹ M, about 5×10⁻¹⁰ M, or about 10⁻¹⁰ M. In some embodiments, anti-ILT7 antibodies or antigen-binding fragments described herein bind to human ILT7 with a K_{D} of about 10⁻⁷ M. In some embodiments, anti-ILT7 antibodies or antigen-binding fragments described herein bind to human ILT7 with a K_{D} ranging from 10⁻¹⁰ M to 10⁻⁶ M, from 10⁻⁹ M to 10⁻⁶ M, from 10⁻⁸ M 10⁻⁶ M, from 10⁻⁷ M to 10⁻⁶ M, from 10⁻¹⁰ M to 5×10⁻⁷ M, from 10⁻⁹ M to 5×10⁻⁷ M, from 10⁻⁸ M to 5×10⁻⁷ M, or from 10⁻⁷ M to 5×10⁻⁷ M. In some embodiments, anti-ILT7 antibodies or antigen-binding fragments described herein bind to human ILT7 with high affinity, for example, with a K_{D} from 10⁻⁷ M to 10⁻⁶ M. In some embodiments, the K_{D} is determined by BLI. In some embodiments, the K_{D} is determined by SPR. In some embodiments, anti-ILT7 antibodies or antigen-binding fragments described herein bind to human ILT7 with high affinity, for example, with a K_{D} of 10⁻⁶ M or less, 5×10⁻⁷ M or less, 10⁻⁷ M or less, 5×10⁻⁸ M or less, 10⁻⁸ M or less, 5×10⁻⁹ M or less, 10⁻⁹ M or less, 5×10⁻¹⁰ M or less, or 10⁻¹⁰ M or less; or ranging from 10⁻¹⁰ M to 10⁻⁶ M, from 10⁻⁹ M to 10⁻⁶ M, from 10⁻⁸ M 10⁻⁶ M, from 10⁻⁷ M to 10⁻⁶ M, from 10⁻¹⁰ M to 5×10⁻⁷ M, from 10⁻⁹ M to 5×10⁻⁷ M, from 10⁻⁷ M to 5×10⁻⁷ M, or from 10⁻⁷ M to 5×10⁻⁷ M, as measured by SPR.

In some embodiments, the anti-ILT7 antibodies or antigen-binding fragments described herein bind to both human ILT7 and cynomolgus ILT7.

In some embodiments, anti-ILT7 antibodies or antigen-binding fragments described herein do not bind to other LILR family members. In some embodiments, the affinities of the anti-ILT7 antibodies or antigen-binding fragments described herein to other LILR family member proteins are comparable to those of an isotype antibody (e.g., hIgG1). Other LILR family member proteins include, but are not limited to: LILRA1, LILRA2/ILT1, LILRA3/ILT6, LILRA5/ILT11, LILRA6/ILT8, LILRB1/ILT2, LILRB2/ILT4, LILRB3/ILT5, LILRB4/ILT3, and LILB5. In some embodiments, anti-ILT7 antibodies or antigen-binding fragments described herein do not bind to one or more of LILR family member proteins selected from the group consisting of LILRA1, LILRA2/ILT1, LILRA3/ILT6, LILRA5/ILT11, LILRA6/ILT8, LILRB1/ILT2, LILRB2/ILT4, LILRB3/ILT5, LILRB4/ILT3, and LILB5. In some embodiments, the anti-ILT7 antibody or antigen-binding fragment described herein does not bind to any of LILR family member proteins: LILRA1, LILRA2/ILT1, LILRA3/ILT6, LILRA5/ILT11, LILRA6/ILT8, LILRB1/ILT2, LILRB2/ILT4, LILRB3/ILT5, LILRB4/ILT3, and LILB5.

In some embodiments, anti-ILT7 antibodies or antigen-binding fragments described herein inhibit IFNα release by PBMCs. In some embodiments, anti-ILT7 antibodies or antigen-binding fragments described herein inhibit IFNα release by CpG-stimulated PBMCs in vitro. In some embodiments, anti-ILT7 antibodies or antigen-binding fragments described herein inhibit IFNα release by PBMCs in vivo. In some embodiments, the anti-ILT7 antibodies or antigen-binding fragments described herein reduce IFNα level in vivo. In some embodiments, anti-ILT7 antibodies or antigen-binding fragments described herein inhibit IFNα release by CpG-stimulated PBMCs in vitro with an EC₅₀ of 10 nM or less, 5 nM or less, 1 nM or less, 0.5 nM or less, 0.1 nM or less, 0.08 nM or less, 0.05 nM or less, or 0.01 nM or less. In some embodiments, anti-ILT7 antibodies or antigen-binding fragments described herein inhibit IFNα release by CpG-stimulated PBMCs in vitro with an EC₅₀ of 0.1 nM or less. In some embodiments, anti-ILT7 antibodies or antigen-binding fragments described herein inhibit IFNα release by CpG-stimulated PBMCs in vitro with an EC₅₀ of about 10 nM, about 5 nM, about 1 nM, about 0.5 nM, about 0.1 nM, about 0.08 nM, about 0.05 nM, or about 0.01 nM. In some embodiments, anti-ILT7 antibodies or antigen-binding fragments described herein inhibit IFNα release by CpG-stimulated PBMCs in vitro with an EC₅₀ of about 0.05 nM. In some embodiments, anti-ILT7 antibodies or antigen-binding fragments described herein inhibit IFNα release by CpG-stimulated PBMCs in vitro with an EC₅₀ ranging from 0.01 nM to 10 nM, from 0.01 nM to 5 nM, from 0.01 nM to 1 nM, from 0.01 nM to 0.5 nM, from 0.01 nM to 0.1 nM, or from 0.01 nM to 0.05 nM. In some embodiments, anti-ILT7 antibodies or antigen-binding fragments described herein inhibit IFNα release by CpG-stimulated PBMCs in vitro with an EC₅₀ from 0.01 nM to 0.1 nM. In some embodiments, anti-ILT7 antibodies or antigen-binding fragments described herein inhibit IFNα release by CpG-stimulated PBMCs in vitro with an EC₅₀ from 0.01 nM to 1 nM. In some embodiments, anti-ILT7 antibodies or antigen-binding fragments described herein inhibit IFNα release by CpG-stimulated PBMCs in vitro with an EC₅₀ that is 60% or less, 50% or less, 40% or less, or 30% or less, of that of the reference antibody daxdilimab. In some embodiments, anti-ILT7 antibodies or antigen-binding fragments described herein inhibit IFNα release by CpG-stimulated PBMCs in vitro with an EC₅₀ that is 50% or less of that of the reference antibody daxdilimab. In some embodiments, anti-ILT7 antibodies or antigen-binding fragments described herein inhibit IFNα release by CpG-stimulated PBMCs in vitro with an EC₅₀ that is 10-60%, 10-50%, 10-40%, 20-60%, 20-50%, or 20-40% of that of the reference antibody daxdilimab. In some embodiments, anti-ILT7 antibodies or antigen-binding fragments described herein inhibit IFNα release by CpG-stimulated PBMCs in vitro with an EC₅₀ that is 10⁻⁵⁰% of that of the reference antibody daxdilimab.

In some embodiments, anti-ILT7 antibodies or antigen-binding fragments described herein bind selectively to pDCs in human PBMCs. In some embodiments, anti-ILT7 antibodies or antigen-binding fragments described herein do not bind to T cells, B cells, NK cells, NKT cells, or monocytes in PBMCs.

In some embodiments, anti-ILT7 antibodies or antigen-binding fragments described herein exhibit ADCC activities and ADCP activities against ILT7-expressing cells, such as pDCs. The ADCC activity can be NK-dependent ADCC. The ADCC activity can be neutrophil-dependent ADCC. In some embodiments, anti-ILT7 antibodies or antigen-binding fragments described herein deplete pDCs in vivo.

In some embodiments, the anti-ILT7 antibodies or antigen-binding fragments described herein exhibit an NK-dependent ADCC activity with an EC₅₀ of 0.05 nM or less, 0.02 nM or less, 0.01 nM or less, 0.008 nM or less, 0.005 nM or less, 0.002 nM or less, or 0.001 nM or less. In some embodiments, the anti-ILT7 antibodies or antigen-binding fragments described herein exhibit an NK-dependent ADCC activity with an EC₅₀ of 0.01 nM or less. In some embodiments, the anti-ILT7 antibodies or antigen-binding fragments described herein exhibit an NK-dependent ADCC activity with an EC₅₀ of 0.008 nM or less. In some embodiments, the anti-ILT7 antibodies or antigen-binding fragments described herein exhibit an NK-dependent ADCC activity with an EC₅₀ of 0.005 nM or less. In some embodiments, the anti-ILT7 antibodies or antigen-binding fragments described herein exhibit an NK-dependent ADCC activity with an EC₅₀ of about 0.05 nM, about 0.02 nM, about 0.01 nM, about 0.008 nM, about 0.005 nM, about 0.002 nM, or about 0.001 nM. In some embodiments, the anti-ILT7 antibodies or antigen-binding fragments described herein exhibit an NK-dependent ADCC activity with an EC₅₀ of about 0.008 nM. In some embodiments, the anti-ILT7 antibodies or antigen-binding fragments described herein exhibit an NK-dependent ADCC activity with an EC₅₀ of about 0.005 nM. In some embodiments, the anti-ILT7 antibodies or antigen-binding fragments described herein exhibit an NK-dependent ADCC activity with an EC₅₀ ranging from 0.001 nM to 0.05 nM, from 0.001 nM to 0.02 nM, from 0.001 nM to 0.01 nM, from 0.001 nM to 0.05 nM, from 0.005 nM to 0.05 nM, from 0.005 nM to 0.02 nM, from 0.005 nM to 0.01 nM, or from 0.001 nM to 0.05 nM. In some embodiments, the anti-ILT7 antibodies or antigen-binding fragments described herein exhibit an NK-dependent ADCC activity with an EC₅₀ ranging from 0.001 nM to 0.01 nM. In some embodiments, the anti-ILT7 antibodies or antigen-binding fragments described herein exhibit an NK-dependent ADCC activity with an EC₅₀ that is 10-60%, 10-50%, 10-40%, 20-60%, 20-50%, or 20-40% of that of the reference antibody daxdilimab. In some embodiments, anti-ILT7 antibodies or antigen-binding fragments described herein exhibit an NK-dependent ADCC activity with an EC₅₀ that is 10⁻⁵⁰% of that of the reference antibody daxdilimab. In some embodiments, anti-ILT7 antibodies or antigen-binding fragments described herein exhibit an NK-dependent ADCC activity with an EC₅₀ that is about 40% of that of the reference antibody daxdilimab.

In some embodiments, the anti-ILT7 antibodies or antigen-binding fragments described herein exhibit a neutrophil-dependent ADCC activity. In some embodiments, the anti-ILT7 antibodies or antigen-binding fragments described herein exhibit a neutrophil-dependent ADCC activity with an EC₅₀ of 500 nM or less, 200 nM or less, 100 nM or less, 80 nM or less, 50 nM or less, 20 nM or less, 10 nM or less, 5 nM or less, or 1 nM or less. In some embodiments, the anti-ILT7 antibodies or antigen-binding fragments described herein exhibit a neutrophil-dependent ADCC activity with an EC₅₀ of 100 nM or less. In some embodiments, the anti-ILT7 antibodies or antigen-binding fragments described herein exhibit a neutrophil-dependent ADCC activity with an EC₅₀ of 50 nM or less. In some embodiments, the anti-ILT7 antibodies or antigen-binding fragments described herein exhibit a neutrophil-dependent ADCC activity with an EC₅₀ ranging from 1 nM to 500 nM, from 1 nM to 200 nM, from 1 nM to 100 nM, from 1 nM to 80 nM, from 1 nM to 50 nM, from 1 nM to 20 nM, from 5 nM to 500 nM, from 5 nM to 200 nM, from 5 nM to 100 nM, from 5 nM to 80 nM, from 5 nM to 50 nM, or from 5 nM to 20 nM. In some embodiments, the anti-ILT7 antibodies or antigen-binding fragments described herein exhibit a neutrophil-dependent ADCC activity with an EC₅₀ ranging from 1 nM to 50 nM. In some embodiments, the anti-ILT7 antibodies or antigen-binding fragments described herein exhibit a neutrophil-dependent ADCC activity with an EC₅₀ of about 500 nM, about 200 nM, about 100 nM, about 80 nM, about 50 nM, about 20 nM, about 10 nM, about 5 nM, or about 1 nM. In some embodiments, the anti-ILT7 antibodies or antigen-binding fragments described herein exhibit a neutrophil-dependent ADCC activity with an EC₅₀ of about 10 nM.

In some embodiments, the anti-ILT7 antibodies or antigen-binding fragments described herein exhibit a macrophage-dependent ADCP activity. In some embodiments, the anti-ILT7 antibodies or antigen-binding fragments described herein exhibit a macrophage-dependent ADCP activity with an EC₅₀ of 10 nM or less, 8 nM or less, 5 nM or less, 2 nM or less, 1 nM or less, 0.5 nM or less, 0.2 nM or less, or 0.1 nM or less. In some embodiments, the anti-ILT7 antibodies or antigen-binding fragments described herein exhibit a macrophage-dependent ADCP activity with an EC₅₀ of 10 nM or less. In some embodiments, the anti-ILT7 antibodies or antigen-binding fragments described herein exhibit a macrophage-dependent ADCP activity with an EC₅₀ of 8 nM or less. In some embodiments, the anti-ILT7 antibodies or antigen-binding fragments described herein exhibit a macrophage-dependent ADCP activity with an EC₅₀ of about 10 nM, about 8 nM, about 5 nM, about 2 nM, about 1 nM, about 0.5 nM, about 0.2 nM, or about 0.1 nM. In some embodiments, the anti-ILT7 antibodies or antigen-binding fragments described herein exhibit a macrophage-dependent ADCP activity with an EC₅₀ of about 1 nM. In some embodiments, the anti-ILT7 antibodies or antigen-binding fragments described herein exhibit a macrophage-dependent ADCP activity with an EC₅₀ ranging from 0.01 to 100 nM, from 0.01 to 50 nM, from 0.01 to 10 nM, from 0.1 to 100 nM, from 0.1 to 50 nM, from 0.1 to 10 nM, or from 0.1 to 5 nM. In some embodiments, the anti-ILT7 antibodies or antigen-binding fragments described herein exhibit a macrophage-dependent ADCP activity with an EC₅₀ ranging from 0.1 to 10 nM. In some embodiments, the anti-ILT7 antibodies or antigen-binding fragments described herein exhibit a macrophage-dependent ADCP activity with an EC₅₀ ranging from 0.5 to 5 nM.

In some embodiments, the anti-ILT7 antibodies or antigen-binding fragments described herein exhibit a maximum phagocytic index of at least 20%, at least 30%, at least 40%, at least 50%, at least 60%, at least 70%, or at least 80%. In some embodiments, the anti-ILT7 antibodies or antigen-binding fragments described herein exhibit a maximum phagocytic index of about 20%, about 30%, about 40%, about 50%, about 60%, about 70%, or about 80%. In some embodiments, the anti-ILT7 antibodies or antigen-binding fragments described herein exhibit a maximum phagocytic index ranging from 20 to 80%, from 20 to 70%, from 20 to 60%, from 30 to 80%, from 30 to 70%, or from 30 to 60%. In some embodiments, the anti-ILT7 antibodies or antigen-binding fragments described herein exhibit a maximum phagocytic index ranging from 20 to 80%. In some embodiments, the anti-ILT7 antibodies or antigen-binding fragments described herein exhibit a maximum phagocytic index ranging from 30 to 60%.

### C. Variants and Conjugates

The present disclosure further contemplates additional variants and equivalents that are substantially homologous to the recombinant, monoclonal, chimeric, humanized, and human antibodies, or antibody fragments thereof, described herein. In some embodiments, it is desirable to improve the binding affinity of the antibody. In some embodiments, it is desirable to modulate biological properties of the antibody, including but not limited to, specificity, thermostability, expression level, effector function(s), glycosylation, immunogenicity, and/or solubility. Those skilled in the art will appreciate that amino acid changes may alter post-translational processes of an antibody, such as changing the number or position of glycosylation sites or altering membrane anchoring characteristics.

Variations can be a substitution, deletion, or insertion of one or more nucleotides encoding the antibody or polypeptide that results in a change in the amino acid sequence as compared with the native antibody or polypeptide sequence. In some embodiments, amino acid substitutions are the result of replacing one amino acid with another amino acid having similar structural and/or chemical properties, such as the replacement of a leucine with a serine, e.g., conservative amino acid replacements. Insertions or deletions can be in the range of about 1 to 5 amino acids. In some embodiments, the substitution, deletion, or insertion includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the parent molecule. In some embodiments, variations in the amino acid sequence that are biologically useful and/or relevant can be determined by systematically making insertions, deletions, or substitutions in the sequence and testing the resulting variant proteins for activity as compared to the parent protein.

In some embodiments, provided herein are variants of anti-ILT7 antibodies or antigen-binding fragments described herein. In some embodiments, provided herein are variants of anti-ILT7 antibody clone Ab12 (cmAb12 or hu-Ab12). In some embodiments, a variant comprises one to 30 amino acid substitutions, additions, and/or deletions in the parent antibody or antigen-binding fragment. In some embodiments, a variant comprises one to 25 amino acid substitutions, additions, and/or deletions in the parent antibody or antigen-binding fragment. In some embodiments, a variant comprises one to 20 substitutions, additions, and/or deletions in the parent antibody or antigen-binding fragment. In some embodiments, a variant comprises one to 15 substitutions, additions, and/or deletions in the parent antibody or antigen-binding fragment. In some embodiments, a variant comprises one to 10 substitutions, additions, and/or deletions in the parent antibody or antigen-binding fragment. In some embodiments, a variant comprises one to five amino acid substitutions, additions, and/or deletions in the parent antibody or antigen-binding fragment. In some embodiments, a variant comprises one to three amino acid substitutions, additions, and/or deletions in the parent antibody or antigen-binding fragment. In some embodiments, the amino acid substitution(s) is in a CDR of the antibody or antigen-binding fragment. In some embodiments, the amino acid substitution(s) is not in a CDR of the antibody or antigen-binding fragment. In some embodiments, the amino acid substitution(s) is in a framework region of the antibody or antigen-binding fragment. In some embodiments, the amino acid substitutions, additions, and/or deletions are conservative amino acid substitutions.

It is known in the art that the constant region(s) of an antibody mediates several effector functions and these effector functions can vary depending on the isotype of the antibody. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fe receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production.

As known in the art, allotypes are polymorphic markers of an IG subclass that correspond to amino acid changes and are detected serologically by antibody reagents. Among others, the allotypes of the human heavy gamma chains of the IgG are designated as Gm (“gamma marker”). The allotypes G1m, G2m, and G3m are carried by the constant region of the gamma1, gamma2, and gamma3 chains, encoded by the IGHG1, IGHG2, and IGHG3 genes, respectively. The gamma1 chains can express G1m alleles (combinations of G1m allotypes): G1m3; G1m3,1; G1m17,1; G1m17,1,2; G1m17,1,27; Gm17,1,28; and Gm17,1,27,28. The C regions of the G1m3,1; G1m17,1; and G1m17,1,2 chains differ from that of the G1m3 chains by two, three, and four amino acids, respectively. The correspondence between the G1m alleles and IGHG1 alleles is known in the art, e.g., Lefranc, Chapter 26—IMGT® Immunoglobulin Repertoire Analysis and Antibody Humanization, MOLECULAR BIOLOGY OF B CELLS (SECOND EDITION), Academic Press, 2015, Pages 481-514 (Table 7). In the IGHG1 CH1, the lysine at position 120 (K120) in strand G corresponds to the G1m17 allotype. The isoleucine 1103 (strand F) is specific of the gamma1 chain isotype. If an arginine is expressed at position 120 (R120), the simultaneous presence of R120 and 1103 corresponds to the expression of the G1m3 allotype. For the gamma3 and gamma4 isotypes (which also have R120 but T in 103), R120 only corresponds to the expression of the nG1m17 isoallotype (an isoallotype or nGm is detected by antibody reagents that identify this marker as an allotype in one IgG subclass and as an isotype for other subclasses). In the IGHG1 CH3, the aspartate D12 and leucine L14 (strand A) correspond to G1 ml, whereas glutamate E12 and methionine M14 correspond to the nG1 m1 isoallotype. A glycine at position 110 corresponds to G1m2, whereas an alanine does not correspond to any allotype (G1m2-negative chain).

See exemplary allotypes of human IgG1 heavy chain constant region (IgG1 CH) below. In some embodiments, provided herein are IgG1 antibodies having a heavy chain constant region (CH) having at least 85% sequence identity to an amino acid sequence selected from the group consisting of SEQ ID NOs: 31 and 40-44. In some embodiments, the IgG1 antibodies provided herein have a CH having at least 85%, at least 90%, at least 95%, at least 98%, or at least 99% sequence identity to SEQ ID NO: 31. In some embodiments, the IgG1 antibodies provided herein have a CH having at least 85%, at least 90%, at least 95%, at least 98%, or at least 99% sequence identity to SEQ ID NO: 40. In some embodiments, the IgG1 antibodies provided herein have a CH having at least 85%, at least 90%, at least 95%, at least 98%, or at least 99% sequence identity to SEQ ID NO: 41. In some embodiments, the IgG1 antibodies provided herein have a CH having at least 85%, at least 90%, at least 95%, at least 98%, or at least 99% sequence identity to SEQ ID NO: 42. In some embodiments, the IgG1 antibodies provided herein have a CH having at least 85%, at least 90%, at least 95%, at least 98%, or at least 99% sequence identity to SEQ ID NO: 43. In some embodiments, the IgG1 antibodies provided herein have a CH having at least 85%, at least 90%, at least 95%, at least 98%, or at least 99% sequence identity to SEQ ID NO: 44.

| Allotype | Sequences |
|---|---|
| IgG1 allotype 0 - CH | ASTKGPSVFPLAPSSKSTSGGTAALGCLVKDYFPEPVTVSWNSG |
| | ALTSGVHTFPAVLQSSGLYSLSSVVTVPSSSLGTQTYICNVNHK |
| | PSNTKVDKKVEPKSCDKTHTCPPCPAPELLGGPSVFLFPPKPKD |
| | TLMISRTPEVTCVVVDVSHEDPEVKFNWYVDGVEVHNAKTKP |
| | REEQYNSTYRVVSVLTVLHQDWLNGKEYKCKVSNKALPAPIE |
| | KTISKAKGQPREPQVYTLPPSRDELTKNQVSLTCLVKGFYPSDI |
| | AVEWESNGQPENNYKTTPPVLDSDGSFFLYSKLTVDKSRWQQ |
| | GNVFSCSVMHEALHNHYTQKSLSLSPGK |
| | (SEQ ID NO: 31) |
| IgG1 allotype 1 - CH | ASTKGPSVFPLAPSSKSTSGGTAALGCLVKDYFPEPVTVSWNSG |
| | ALTSGVHTFPAVLQSSGLYSLSSVVTVPSSSLGTQTYICNVNHK |
| | PSNTKVDKRVEPKSCDKTHTCPPCPAPELLGGPSVFLFPPKPKD |
| | TLMISRTPEVTCVVVDVSHEDPEVKFNWYVDGVEVHNAKTKP |
| | REEQYNSTYRVVSVLTVLHQDWLNGKEYKCKVSNKALPAPIE |
| | KTISKAKGQPREPQVYTLPPSRDELTKNQVSLTCLVKGFYPSDI |
| | AVEWESNGQPENNYKTTPPVLDSDGSFFLYSKLTVDKSRWQQ |
| | GNVFSCSVMHEALHNHYTQKSLSLSPGK |
| | (SEQ ID NO: 40) |
| IgG1 allotype 2 - CH | ASTKGPSVFPLAPSSKSTSGGTAALGCLVKDYFPEPVTVSWNSG |
| | ALTSGVHTFPAVLQSSGLYSLSSVVTVPSSSLGTQTYICNVNHK |
| | PSNTKVDKRVEPKSCDKTHTCPPCPAPELLGGPSVFLFPPKPKD |
| | TLMISRTPEVTCVVVDVSHEDPEVKFNWYVDGVEVHNAKTKP |
| | REEQYNSTYRVVSVLTVLHQDWLNGKEYKCKVSNKALPAPIE |
| | KTISKAKGQPREPQVYTLPPSREEMTKNQVSLTCLVKGFYPSDI |
| | AVEWESNGQPENNYKTTPPVLDSDGSFFLYSKLTVDKSRWQQ |
| | GNVFSCSVMHEALHNHYTQKSLSLSPGK |
| | (SEQ ID NO: 41) |
| IgG1 allotype 3 - CH | ASTKGPSVFPLAPSSKSTSGGTAALGCLVKDYFPEPVTVSWNSG |
| | ALTSGVHTFPAVLQSSGLYSLSSVVTVPSSSLGTQTYICNVNHK |
| | PSNTKVDKKVEPKSCDKTHTCPPCPAPELLGGPSVFLFPPKPKD |
| | TLMISRTPEVTCVVVDVSHEDPEVKFNWYVDGVEVHNAKTKP |
| | REEQYNSTYRVVSVLTVLHQDWLNGKEYKCKVSNKALPAPIE |
| | KTISKAKGQPREPQVYTLPPSRDELTKNQVSLTCLVKGFYPSDI |
| | AVEWESNGQPENNYKTTPPVLDSDGSFFLYSKLTVDKSRWQQ |
| | GNIFSCSVMHEALHNHYTQKSLSLSPGK |
| | (SEQ ID NO: 42) |
| IgG1 allotype 4 - CH | ASTKGPSVFPLAPSSKSTSGGTAALGCLVKDYFPEPVTVSWNSG |
| | ALTSGVHTFPAVLQSSGLYSLSSVVTVPSSSLGTQTYICNVNHK |
| | PSNTKVDKKVEPKSCDKTHTCPPCPAPELLGGPSVFLFPPKPKD |
| | TLMISRTPEVTCVVVDVSHEDPEVKFNWYVDGVEVHNAKTKP |
| | REEQYNSTYRVVSVLTVLHQDWLNGKEYKCKVSNKALPAPIE |
| | KTISKAKGQPREPQVYTLPPSRDELTKNQVSLTCLVKGFYPSDI |
| | AVEWESNGQPENNYKTTPPVLDSDGSFFLYSKLTVDKSRWQQ |
| | GNVFSCSVMHEALHNRYTQKSLSLSPGK |
| | (SEQ ID NO: 43) |
| IgG1 allotype 5 - CH | ASTKGPSVFPLAPSSKSTSGGTAALGCLVKDYFPEPVTVSWNSG |
| | ALTSGVHTFPAVLQSSGLYSLSSVVTVPSSSLGTQTYICNVNHK |
| | PSNTKVDKKVEPKSCDKTHTCPPCPAPELLGGPSVFLFPPKPKD |
| | TLMISRTPEVTCVVVDVSHEDPEVKFNWYVDGVEVHNAKTKP |
| | REEQYNSTYRVVSVLTVLHQDWLNGKEYKCKVSNKALPAPIE |
| | KTISKAKGQPREPQVYTLPPSRDELTKNQVSLTCLVKGFYPSDI |
| | AVEWESNGQPENNYKTTPPVLDSDGSFFLYSKLTVDKSRWQQ |
| | GNVFSCSVMHEGLHNHTTQKSLSLSPGK |
| | (SEQ ID NO: 44 ) |

The gamma2 chains can express G2m alleles. Position 45.1 (first position of the transversal CD strand) corresponds to the presence of the only identified G2m allotype (G2m23) or to its absence (G2m . . . ). Valine V45.1 corresponds to G2m . . . , whereas a methionine would correspond to G2m23.

The gamma3 chains can express G3m alleles (combinations of G3m allotypes). G3m16 (W83) and G3m21 (L82), nG3m21 (P82) are located on the CH2. The other G3m allotypes form two mosaics on the CH3. G3m26 (R115), G3m5 (R115, F116), G3m28 (R115, Y116), nG3m5 (H115, Y116), G3m14 (M84, R115, F116) and G3m15 (M39, H115, Y116) form a first mosaic. G3m11 (S44), nG3m11 (N44), G3m10 (S44, I101), G3m24 (S44, V101), G3m27 (I101), G3m6 (S44, E98), G3m13 (S44, Q98) form a second mosaic.

In some embodiments, anti-ILT7 antibody or antigen-binding fragment described herein comprise a constant region of a human IgA antibody. In some embodiments, anti-ILT7 antibody or antigen-binding fragment described herein comprise a constant region of a human IgD antibody. In some embodiments, anti-ILT7 antibody or antigen-binding fragment described herein comprise a constant region of a human IgE antibody. In some embodiments, anti-ILT7 antibody or antigen-binding fragment described herein comprise a constant region of a human IgG antibody. In some embodiments, anti-ILT7 antibody or antigen-binding fragment described herein comprise a constant region of a human IgM antibody. In some embodiments, anti-ILT7 antibody or antigen-binding fragment described herein comprise a constant region of a human IgG1 antibody. In some embodiments, anti-ILT7 antibody or antigen-binding fragment described herein comprise a constant region of a human IgG2 antibody. In some embodiments, anti-ILT7 antibody or antigen-binding fragment described herein comprise a constant region of a human IgG3 antibody. In some embodiments, anti-ILT7 antibody or antigen-binding fragment described herein comprise a constant region of a human IgG4 antibody. In some embodiments, anti-ILT7 antibody or antigen-binding fragment described herein comprise a constant region of a human IgG1 antibody, wherein the IgG1 antibody can be of any allotype known in the art. In some embodiments, the IgG1 antibody is of allotype G1m3; G1m3,1; G1m17,1; G1m17,1,2; G1m17,1,27; Gm17,1,28; or Gm17,1,27,28. In some embodiments, the IgG1 antibody is of allotype G1m3. In some embodiments, the IgG1 antibody is of allotype G1m3,1. In some embodiments, the IgG1 antibody is of allotype G1m17,1. In some embodiments, the IgG1 antibody is of allotype G1m17,1,2. In some embodiments, the IgG1 antibody is of allotype Gm17,1,28. In some embodiments, the IgG1 antibody is of allotype Gm17,1,27,28. In some embodiments, anti-ILT7 antibody or antigen-binding fragment described herein comprise a constant region of a human IgG2 antibody, wherein the IgG2 antibody can be of any allotype known in the art. In some embodiments, the IgG2 antibody is of allotype G2m23. In some embodiments, the IgG2 antibody is of allotype G2m . . . . In some embodiments, anti-ILT7 antibody or antigen-binding fragment described herein comprise a constant region of a human IgG3 antibody, wherein the IgG3 antibody can be of any allotype known in the art. In some embodiments, the IgG3 antibody is of allotype G3m16, G3m21, G3m26, G3m5, G3m28, G3m14, G3m15, G3 m11, G3m10, G3m24, G3m27, G3m6, or G3m13. In some embodiments, the IgG3 antibody is of allotype G3m16. In some embodiments, the IgG3 antibody is of allotype G3m21. In some embodiments, the IgG3 antibody is of allotype G3m26. In some embodiments, the IgG3 antibody is of allotype G3m5. In some embodiments, the IgG3 antibody is of allotype G3m28. In some embodiments, the IgG3 antibody is of allotype G3m14. In some embodiments, the IgG3 antibody is of allotype G3m15. In some embodiments, the IgG3 antibody is of allotype G3m11. In some embodiments, the IgG3 antibody is of allotype G3m10. In some embodiments, the IgG3 antibody is of allotype G3m24. In some embodiments, the IgG3 antibody is of allotype G3m27. In some embodiments, the IgG3 antibody is of allotype G3m6. In some embodiments, the IgG3 antibody is of allotype G3m13.

In some embodiments, at least one or more of the constant regions has been modified or deleted in the anti-ILT7 antibody or antigen-binding fragment described herein. In some embodiments, the antibodies comprise modifications to one or more of the three heavy chain constant regions (CH1, CH2 or CH3) and/or to the light chain constant region (CL).

In some embodiments, the heavy chain constant region of the modified antibodies comprises at least one human constant region. In some embodiments, the heavy chain constant region of the modified antibodies comprises more than one human constant region. In some embodiments, modifications to the constant region comprise additions, deletions, or substitutions of one or more amino acids in one or more regions. In some embodiments, one or more regions are partially or entirely deleted from the constant regions of the modified antibodies. In some embodiments, the entire CH2 domain has been removed from an antibody (ΔCH2 constructs). In some embodiments, a deleted constant region is replaced by a short amino acid spacer that provides some of the molecular flexibility typically imparted by the absent constant region. In some embodiments, a modified antibody comprises a CH3 domain directly fused to the hinge region of the antibody. In some embodiments, a modified antibody comprises a peptide spacer inserted between the hinge region and modified CH2 and/or CH3 domains.

In some embodiments, an anti-ILT7 antibody or antigen-binding fragment comprises a Fc region. In some embodiments, the Fc region is fused via a hinge. The hinge can be an IgG1 hinge, an IgG2 hinge, or an IgG3 hinge. The amino acid sequences of the Fc region of human IgG1, IgG2, IgG3, and IgG4 are known to those of ordinary skill in the art. In some cases, Fe regions with amino acid variations have been identified in native antibodies. In some embodiments, the modified antibodies (e.g., modified Fe region) provide for altered effector functions that, in turn, affect the biological profile of the antibody. For example, in some embodiments, the deletion or inactivation (through point mutations or other means) of a constant region reduces Fc receptor binding of the modified antibody as it circulates. In some embodiments, the constant region modifications reduce the immunogenicity of the antibody. In some embodiments, the constant region modifications increase the serum half-life of the antibody. In some embodiments, the constant region modifications reduce the serum half-life of the antibody. In some embodiments, the constant region modifications enhance ADCC and/or complement dependent cytotoxicity (CDC) of the antibody. In some embodiments, the constant region modifications enhance antibody-dependent cellular phagocytosis (ADCP) of the antibody. In some embodiments, the constant region modifications decrease or remove ADCC and/or CDC of the antibody. In some embodiments, specific amino acid substitutions in a human IgG1 Fe region with corresponding IgG2 or IgG4 residues reduce effector functions (e.g., ADCC and CDC) in the modified antibody. In some embodiments, an antibody does not have one or more effector functions (e.g., “effectorless” antibodies). In some embodiments, the antibody does not bind an Fc receptor and/or complement factors. In some embodiments, the antibody has no effector function(s). In some embodiments, the constant region modifications increase or enhance ADCC and/or ADCP of the antibody. In some embodiments, the constant region is modified to eliminate disulfide linkages or oligosaccharide moieties. In some embodiments, the constant region is modified to add/substitute one or more amino acids to provide one or more cytotoxin, oligosaccharide, or carbohydrate attachment sites. In some embodiments, an anti-ILT7 antibody or antigen-binding fragment comprises a variant Fc region that is engineered with substitutions at specific amino acid positions as compared to a native Fc region.

In some embodiments, an anti-ILT7 antibody or antigen-binding fragment described herein comprises an IgG1 heavy chain constant region that comprises one or more amino acid substitutions selected from the group consisting of L234, L235, G236, S239, F243, H268, D270, R292, S298, Y300, V305, A330, I332, K326, E333, K334, and P396, numbered according to the EU Index.

In some embodiments, an anti-ILT7 antibody or antigen-binding fragment described herein comprises an IgG1 heavy chain constant region that comprises at least one amino acid substitution. The IgG1 heavy chain constant region can comprise a L234 substitution. The L234 substitution can be, e.g., L234Y. The IgG1 heavy chain constant region can comprise a L235 substitution. The L235 substitution can be, e.g., L235Q or L235V. The IgG1 heavy chain constant region can comprise a G236 substitution. The G236 substitution can be, e.g., G236A or G236W. The IgG1 heavy chain constant region can comprise an S239 substitution. The S239 substitution can be, e.g., S239D or S239M. The IgG1 heavy chain constant region can comprise an F243 substitution. The F243 substitution can be, e.g., F243L. The IgG1 heavy chain constant region can comprise an H268 substitution. The H268 substitution can be, e.g., H268D. The IgG1 heavy chain constant region can comprise a D270 substitution. The D270 substitution can be, e.g., D270E. The IgG1 heavy chain constant region can comprise an R292 substitution. The R292 substitution can be, e.g., R292P. The IgG1 heavy chain constant region can comprise an S298 substitution. The S298 substitution can be, e.g., S298A. The IgG1 heavy chain constant region can comprise a Y300 substitution. The Y300 substitution can be, e.g., Y300L. The IgG1 heavy chain constant region can comprise a V305 substitution. The V305 substitution can be, e.g., V305I. The IgG1 heavy chain constant region can comprise a K326 substitution. The K326 substitution can be, e.g., K326D. The IgG1 heavy chain constant region can comprise an A330 substitution. The A330 substitution can be, e.g., A330M or A330L. The IgG1 heavy chain constant region can comprise an 1332 substitution. The 1332 substitution can be, e.g., I332E. The IgG1 heavy chain constant region can comprise an E333 substitution. The E333 substitution can be, e.g., E333A. The IgG1 heavy chain constant region can comprise a K334 substitution. The K334 substitution can be, e.g., K334A or K334E. The IgG1 heavy chain constant region can comprise a P396 substitution. The P396 substitution can be, e.g., P396L.

In some embodiments, an anti-ILT7 antibody or antigen-binding fragment described herein comprises an IgG1 heavy chain constant region that comprises one or more amino acid substitutions selected from the group consisting of L234Y, L235Q, L235V, G236A, G236W, S239D, S239M, F243L, H268D, D270E, R292P, S298A, Y300L, V305I, K326D, A330M, A330L, I332E, E333A, K334A, K334E, and P396L, numbered according to the EU Index. In some embodiments, the IgG1 heavy chain constant region comprises one or more amino acid substitutions selected from the group consisting of K214R, L234A, L235E, G237A, A330S, P331S, D356E, and L358M, numbered according to the EU Index. In some embodiments, the anti-ILT7 antibodies and antigen-binding fragments described herein comprise a variant of human IgG1 heavy chain constant region modified by amino acid substitutions S298A, E333A, and K334A. In some embodiments, the anti-ILT7 antibodies and antigen-binding fragments described herein comprise a variant of human IgG1 heavy chain constant region modified by amino acid substitutions S239D and I332E. In some embodiments, the anti-ILT7 antibodies and antigen-binding fragments described herein comprise a variant of human IgG1 heavy chain constant region modified by amino acid substitutions S239D, A330L, and I332E. In some embodiments, the anti-ILT7 antibodies and antigen-binding fragments described herein comprise a variant of human IgG1 heavy chain constant region modified by amino acid substitution G236A. In some embodiments, the anti-ILT7 antibodies and antigen-binding fragments described herein comprise a variant of human IgG1 heavy chain constant region modified by amino acid substitutions G236A, S239D, and I332E. In some embodiments, the anti-ILT7 antibodies and antigen-binding fragments described herein comprise a variant of human IgG1 heavy chain constant region modified by amino acid substitutions G236A, A330L, and I332E. In some embodiments, the anti-ILT7 antibodies and antigen-binding fragments described herein comprise a variant of human IgG1 heavy chain constant region modified by amino acid substitutions G236A, S239D, A330L, and I332E. In some embodiments, the anti-ILT7 antibodies and antigen-binding fragments described herein comprise a variant of human IgG1 heavy chain constant region modified by amino acid substitutions F243L, R292P, Y300L, V305I, and P396L. In some embodiments, the anti-ILT7 antibodies and antigen-binding fragments described herein comprise a variant of human IgG1 heavy chain constant region modified by amino acid substitutions L235V, F243L, R292P, Y300L, and P396L. In some embodiments, the anti-ILT7 antibodies and antigen-binding fragments described herein comprise a variant of human IgG1 heavy chain constant region modified by amino acid substitutions L234Y, L235Q, G236W, S239M, H268D, D270E, and S298A. In some embodiments, the anti-ILT7 antibodies and antigen-binding fragments described herein comprise a variant of human IgG1 heavy chain constant region modified by amino acid substitutions D270E, K326D, A330M, and K334E. All are numbered according to the EU Index. Provided below are the heavy chain constant region (CH) of exemplary IgG1 allotypes with different mutations that increase or enhance ADCC and/or ADCP of the antibody. Expressly contemplated for inclusion into antibodies disclosed herein are heavy chain constant region (CH) of any immunoglobulin (e.g., human IgG1) disclosed herein or otherwise known in the art with any combinations of mutations disclosed herein or otherwise known in the art for increasing or enhancing ADCC and/or ADCP of the antibody.

| IgG1 Heavy Chain | |
|---|---|
| Constant Region | Sequences |
| IgG1 allotype 0- | ASTKGPSVFPLAPSSKSTSGGTAALGCLVKDYFPEPVTVSWNSG |
| AAA | ALTSGVHTFPAVLQSSGLYSLSSVVTVPSSSLGTQTYICNVNHK |
| | PSNTKVDKKVEPKSCDKTHTCPPCPAPELLGGPSVFLFPPKPKD |
| | TLMISRTPEVTCVVVDVSHEDPEVKFNWYVDGVEVHNAKTKP |
| | REEQYNATYRVVSVLTVLHQDWLNGKEYKCKVSNKALPAPIA |
| | ATISKAKGQPREPQVYTLPPSRDELTKNQVSLTCLVKGFYPSDI |
| | AVEWESNGQPENNYKTTPPVLDSDGSFFLYSKLTVDKSRWQQ |
| | GNVFSCSVMHEALHNHYTQKSLSLSPGK |
| | (SEQ ID NO: 45) |
| IgG1 allotype 0-DE | ASTKGPSVFPLAPSSKSTSGGTAALGCLVKDYFPEPVTVSWNSG |
| | ALTSGVHTFPAVLQSSGLYSLSSVVTVPSSSLGTQTYICNVNHK |
| | PSNTKVDKKVEPKSCDKTHTCPPCPAPELLGGPDVFLFPPKPKD |
| | TLMISRTPEVTCVVVDVSHEDPEVKFNWYVDGVEVHNAKTKP |
| | REEQYNSTYRVVSVLTVLHQDWLNGKEYKCKVSNKALPAPEE |
| | KTISKAKGQPREPQVYTLPPSRDELTKNQVSLTCLVKGFYPSDI |
| | AVEWESNGQPENNYKTTPPVLDSDGSFFLYSKLTVDKSRWQQ |
| | GNVFSCSVMHEALHNHYTQKSLSLSPGK |
| | (SEQ ID NO: 46) |
| IgG1 allotype 0- | ASTKGPSVFPLAPSSKSTSGGTAALGCLVKDYFPEPVTVSWNSG |
| DLE | ALTSGVHTFPAVLQSSGLYSLSSVVTVPSSSLGTQTYICNVNHK |
| | PSNTKVDKKVEPKSCDKTHTCPPCPAPELLGGPDVFLFPPKPKD |
| | TLMISRTPEVTCVVVDVSHEDPEVKFNWYVDGVEVHNAKTKP |
| | REEQYNSTYRVVSVLTVLHQDWLNGKEYKCKVSNKALPLPEE |
| | KTISKAKGQPREPQVYTLPPSRDELTKNQVSLTCLVKGFYPSDI |
| | AVEWESNGQPENNYKTTPPVLDSDGSFFLYSKLTVDKSRWQQ |
| | GNVFSCSVMHEALHNHYTQKSLSLSPGK |
| | (SEQ ID NO: 47) |
| IgG1 allotype 0- | ASTKGPSVFPLAPSSKSTSGGTAALGCLVKDYFPEPVTVSWNSG |
| G236A | ALTSGVHTFPAVLQSSGLYSLSSVVTVPSSSLGTQTYICNVNHK |
| | PSNTKVDKKVEPKSCDKTHTCPPCPAPELLAGPSVFLFPPKPKD |
| | TLMISRTPEVTCVVVDVSHEDPEVKFNWYVDGVEVHNAKTKP |
| | REEQYNSTYRVVSVLTVLHQDWLNGKEYKCKVSNKALPAPIE |
| | KTISKAKGQPREPQVYTLPPSRDELTKNQVSLTCLVKGFYPSDI |
| | AVEWESNGQPENNYKTTPPVLDSDGSFFLYSKLTVDKSRWQQ |
| | GNVFSCSVMHEALHNHYTQKSLSLSPGK |
| | (SEQ ID NO: 48) |
| IgG1 allotype 0- | ASTKGPSVFPLAPSSKSTSGGTAALGCLVKDYFPEPVTVSWNSG |
| LPLIL | ALTSGVHTFPAVLQSSGLYSLSSVVTVPSSSLGTQTYICNVNHK |
| | PSNTKVDKKVEPKSCDKTHTCPPCPAPELLGGPSVFLLPPKPKD |
| | TLMISRTPEVTCVVVDVSHEDPEVKFNWYVDGVEVHNAKTKP |
| | PEEQYNSTLRVVSILTVLHQDWLNGKEYKCKVSNKALPAPIEK |
| | TISKAKGQPREPQVYTLPPSRDELTKNQVSLTCLVKGFYPSDIA |
| | VEWESNGQPENNYKTTPLVLDSDGSFFLYSKLTVDKSRWQQG |
| | NVFSCSVMHEALHNHYTQKSLSLSPGK |
| | (SEQ ID NO: 49) |
| IgG1 allotype 1- | ASTKGPSVFPLAPSSKSTSGGTAALGCLVKDYFPEPVTVSWNSG |
| AAA | ALTSGVHTFPAVLQSSGLYSLSSVVTVPSSSLGTQTYICNVNHK |
| | PSNTKVDKRVEPKSCDKTHTCPPCPAPELLGGPSVFLFPPKPKD |
| | TLMISRTPEVTCVVVDVSHEDPEVKFNWYVDGVEVHNAKTKP |
| | REEQYNATYRVVSVLTVLHQDWLNGKEYKCKVSNKALPAPIA |
| | ATISKAKGQPREPQVYTLPPSRDELTKNQVSLTCLVKGFYPSDI |
| | AVEWESNGQPENNYKTTPPVLDSDGSFFLYSKLTVDKSRWQQ |
| | GNVFSCSVMHEALHNHYTQKSLSLSPGK |
| | (SEQ ID NO: 50) |
| IgG1 allotype 1- | ASTKGPSVFPLAPSSKSTSGGTAALGCLVKDYFPEPVTVSWNSG |
| DLE | ALTSGVHTFPAVLQSSGLYSLSSVVTVPSSSLGTQTYICNVNHK |
| | PSNTKVDKRVEPKSCDKTHTCPPCPAPELLGGPDVFLFPPKPKD |
| | TLMISRTPEVTCVVVDVSHEDPEVKFNWYVDGVEVHNAKTKP |
| | REEQYNSTYRVVSVLTVLHQDWLNGKEYKCKVSNKALPLPEE |
| | KTISKAKGQPREPQVYTLPPSRDELTKNQVSLTCLVKGFYPSDI |
| | AVEWESNGQPENNYKTTPPVLDSDGSFFLYSKLTVDKSRWQQ |
| | GNVFSCSVMHEALHNHYTQKSLSLSPGK |
| | (SEQ ID NO: 51) |
| IgG1 allotype 1- | ASTKGPSVFPLAPSSKSTSGGTAALGCLVKDYFPEPVTVSWNSG |
| ALE | ALTSGVHTFPAVLQSSGLYSLSSVVTVPSSSLGTQTYICNVNHK |
| | PSNTKVDKRVEPKSCDKTHTCPPCPAPELLAGPSVFLFPPKPKD |
| | TLMISRTPEVTCVVVDVSHEDPEVKFNWYVDGVEVHNAKTKP |
| | REEQYNSTYRVVSVLTVLHQDWLNGKEYKCKVSNKALPLPEE |
| | KTISKAKGQPREPQVYTLPPSRDELTKNQVSLTCLVKGFYPSDI |
| | AVEWESNGQPENNYKTTPPVLDSDGSFFLYSKLTVDKSRWQQ |
| | GNVFSCSVMHEALHNHYTQKSLSLSPGK |
| | (SEQ ID NO: 52) |
| IgG1 allotype 2- | ASTKGPSVFPLAPSSKSTSGGTAALGCLVKDYFPEPVTVSWNSG |
| AAA | ALTSGVHTFPAVLQSSGLYSLSSVVTVPSSSLGTQTYICNVNHK |
| | PSNTKVDKRVEPKSCDKTHTCPPCPAPELLGGPSVFLFPPKPKD |
| | TLMISRTPEVTCVVVDVSHEDPEVKFNWYVDGVEVHNAKTKP |
| | REEQYNATYRVVSVLTVLHQDWLNGKEYKCKVSNKALPAPIA |
| | ATISKAKGQPREPQVYTLPPSREEMTKNQVSLTCLVKGFYPSDI |
| | AVEWESNGQPENNYKTTPPVLDSDGSFFLYSKLTVDKSRWQQ |
| | GNVFSCSVMHEALHNHYTQKSLSLSPGK |
| | (SEQ ID NO: 53) |
| IgG1 allotype 2- | ASTKGPSVFPLAPSSKSTSGGTAALGCLVKDYFPEPVTVSWNSG |
| G236A | ALTSGVHTFPAVLQSSGLYSLSSVVTVPSSSLGTQTYICNVNHK |
| | PSNTKVDKRVEPKSCDKTHTCPPCPAPELLAGPSVFLFPPKPKD |
| | TLMISRTPEVTCVVVDVSHEDPEVKFNWYVDGVEVHNAKTKP |
| | REEQYNSTYRVVSVLTVLHQDWLNGKEYKCKVSNKALPAPIE |
| | KTISKAKGQPREPQVYTLPPSREEMTKNQVSLTCLVKGFYPSDI |
| | AVEWESNGQPENNYKTTPPVLDSDGSFFLYSKLTVDKSRWQQ |
| | GNVFSCSVMHEALHNHYTQKSLSLSPGK |
| | (SEQ ID NO: 54) |
| IgG1 allotype 2- | ASTKGPSVFPLAPSSKSTSGGTAALGCLVKDYFPEPVTVSWNSG |
| ADE | ALTSGVHTFPAVLQSSGLYSLSSVVTVPSSSLGTQTYICNVNHK |
| | PSNTKVDKRVEPKSCDKTHTCPPCPAPELLAGPDVFLFPPKPKD |
| | TLMISRTPEVTCVVVDVSHEDPEVKFNWYVDGVEVHNAKTKP |
| | REEQYNSTYRVVSVLTVLHQDWLNGKEYKCKVSNKALPAPEE |
| | KTISKAKGQPREPQVYTLPPSREEMTKNQVSLTCLVKGFYPSDI |
| | AVEWESNGQPENNYKTTPPVLDSDGSFFLYSKLTVDKSRWQQ |
| | GNVFSCSVMHEALHNHYTQKSLSLSPGK |
| | (SEQ ID NO: 55) |
| IgG1 allotype 3- | ASTKGPSVFPLAPSSKSTSGGTAALGCLVKDYFPEPVTVSWNSG |
| AAA | ALTSGVHTFPAVLQSSGLYSLSSVVTVPSSSLGTQTYICNVNHK |
| | PSNTKVDKKVEPKSCDKTHTCPPCPAPELLGGPSVFLFPPKPKD |
| | TLMISRTPEVTCVVVDVSHEDPEVKFNWYVDGVEVHNAKTKP |
| | REEQYNATYRVVSVLTVLHQDWLNGKEYKCKVSNKALPAPIA |
| | ATISKAKGQPREPQVYTLPPSRDELTKNQVSLTCLVKGFYPSDI |
| | AVEWESNGQPENNYKTTPPVLDSDGSFFLYSKLTVDKSRWQQ |
| | GNIFSCSVMHEALHNHYTQKSLSLSPGK |
| | (SEQ ID NO: 56) |
| IgG1 allotype 3- | ASTKGPSVFPLAPSSKSTSGGTAALGCLVKDYFPEPVTVSWNSG |
| ADLE | ALTSGVHTFPAVLQSSGLYSLSSVVTVPSSSLGTQTYICNVNHK |
| | PSNTKVDKKVEPKSCDKTHTCPPCPAPELLAGPDVFLFPPKPKD |
| | TLMISRTPEVTCVVVDVSHEDPEVKFNWYVDGVEVHNAKTKP |
| | REEQYNSTYRVVSVLTVLHQDWLNGKEYKCKVSNKALPLPEE |
| | KTISKAKGQPREPQVYTLPPSRDELTKNQVSLTCLVKGFYPSDI |
| | AVEWESNGQPENNYKTTPPVLDSDGSFFLYSKLTVDKSRWQQ |
| | GNIFSCSVMHEALHNHYTQKSLSLSPGK |
| | (SEQ ID NO: 57) |
| IgG1 allotype 3- | ASTKGPSVFPLAPSSKSTSGGTAALGCLVKDYFPEPVTVSWNSG |
| LPLIL | ALTSGVHTFPAVLQSSGLYSLSSVVTVPSSSLGTQTYICNVNHK |
| | PSNTKVDKKVEPKSCDKTHTCPPCPAPELLGGPSVFLLPPKPKD |
| | TLMISRTPEVTCVVVDVSHEDPEVKFNWYVDGVEVHNAKTKP |
| | PEEQYNSTLRVVSILTVLHQDWLNGKEYKCKVSNKALPAPIEK |
| | TISKAKGQPREPQVYTLPPSRDELTKNQVSLTCLVKGFYPSDIA |
| | VEWESNGQPENNYKTTPLVLDSDGSFFLYSKLTVDKSRWQQG |
| | NIFSCSVMHEALHNHYTQKSLSLSPGK |
| | (SEQ ID NO: 58) |
| IgG1 allotype 4- | ASTKGPSVFPLAPSSKSTSGGTAALGCLVKDYFPEPVTVSWNSG |
| AAA | ALTSGVHTFPAVLQSSGLYSLSSVVTVPSSSLGTQTYICNVNHK |
| | PSNTKVDKKVEPKSCDKTHTCPPCPAPELLGGPSVFLFPPKPKD |
| | TLMISRTPEVTCVVVDVSHEDPEVKFNWYVDGVEVHNAKTKP |
| | REEQYNATYRVVSVLTVLHQDWLNGKEYKCKVSNKALPAPIA |
| | ATISKAKGQPREPQVYTLPPSRDELTKNQVSLTCLVKGFYPSDI |
| | AVEWESNGQPENNYKTTPPVLDSDGSFFLYSKLTVDKSRWQQ |
| | GNVFSCSVMHEALHNRYTQKSLSLSPGK |
| | (SEQ ID NO: 59) |
| IgG1 allotype 4- | ASTKGPSVFPLAPSSKSTSGGTAALGCLVKDYFPEPVTVSWNSG |
| DLE | ALTSGVHTFPAVLQSSGLYSLSSVVTVPSSSLGTQTYICNVNHK |
| | PSNTKVDKKVEPKSCDKTHTCPPCPAPELLGGPDVFLFPPKPKD |
| | TLMISRTPEVTCVVVDVSHEDPEVKFNWYVDGVEVHNAKTKP |
| | REEQYNSTYRVVSVLTVLHQDWLNGKEYKCKVSNKALPLPEE |
| | KTISKAKGQPREPQVYTLPPSRDELTKNQVSLTCLVKGFYPSDI |
| | AVEWESNGQPENNYKTTPPVLDSDGSFFLYSKLTVDKSRWQQ |
| | GNVFSCSVMHEALHNRYTQKSLSLSPGK |
| | (SEQ ID NO: 60) |
| IgG1 allotype 4- | ASTKGPSVFPLAPSSKSTSGGTAALGCLVKDYFPEPVTVSWNSG |
| LPLIL | ALTSGVHTFPAVLQSSGLYSLSSVVTVPSSSLGTQTYICNVNHK |
| | PSNTKVDKKVEPKSCDKTHTCPPCPAPELLGGPSVFLLPPKPKD |
| | TLMISRTPEVTCVVVDVSHEDPEVKFNWYVDGVEVHNAKTKP |
| | PEEQYNSTLRVVSILTVLHQDWLNGKEYKCKVSNKALPAPIEK |
| | TISKAKGQPREPQVYTLPPSRDELTKNQVSLTCLVKGFYPSDIA |
| | VEWESNGQPENNYKTTPLVLDSDGSFFLYSKLTVDKSRWQQG |
| | NVFSCSVMHEALHNRYTQKSLSLSPGK |
| | (SEQ ID NO: 61) |
| IgG1 allotype 5- | ASTKGPSVFPLAPSSKSTSGGTAALGCLVKDYFPEPVTVSWNSG |
| AAA | ALTSGVHTFPAVLQSSGLYSLSSVVTVPSSSLGTQTYICNVNHK |
| | PSNTKVDKKVEPKSCDKTHTCPPCPAPELLGGPSVFLFPPKPKD |
| | TLMISRTPEVTCVVVDVSHEDPEVKFNWYVDGVEVHNAKTKP |
| | REEQYNATYRVVSVLTVLHQDWLNGKEYKCKVSNKALPAPIA |
| | ATISKAKGQPREPQVYTLPPSRDELTKNQVSLTCLVKGFYPSDI |
| | AVEWESNGQPENNYKTTPPVLDSDGSFFLYSKLTVDKSRWQQ |
| | GNVFSCSVMHEGLHNHTTQKSLSLSPGK |
| | (SEQ ID NO: 62) |
| IgG1 allotype 5- | ASTKGPSVFPLAPSSKSTSGGTAALGCLVKDYFPEPVTVSWNSG |
| LPLIL | ALTSGVHTFPAVLQSSGLYSLSSVVTVPSSSLGTQTYICNVNHK |
| | PSNTKVDKKVEPKSCDKTHTCPPCPAPELLGGPSVFLLPPKPKD |
| | TLMISRTPEVTCVVVDVSHEDPEVKFNWYVDGVEVHNAKTKP |
| | PEEQYNSTLRVVSILTVLHQDWLNGKEYKCKVSNKALPAPIEK |
| | TISKAKGQPREPQVYTLPPSRDELTKNQVSLTCLVKGFYPSDIA |
| | VEWESNGQPENNYKTTPLVLDSDGSFFLYSKLTVDKSRWQQG |
| | NVFSCSVMHEGLHNHTTQKSLSLSPGK |
| | (SEQ ID NO: 63) |
| IgG1 allotype 5- | ASTKGPSVFPLAPSSKSTSGGTAALGCLVKDYFPEPVTVSWNSG |
| VLPLL | ALTSGVHTFPAVLQSSGLYSLSSVVTVPSSSLGTQTYICNVNHK |
| | PSNTKVDKKVEPKSCDKTHTCPPCPAPELVGGPSVFLLPPKPKD |
| | TLMISRTPEVTCVVVDVSHEDPEVKFNWYVDGVEVHNAKTKP |
| | PEEQYNSTLRVVSVLTVLHQDWLNGKEYKCKVSNKALPAPIEK |
| | TISKAKGQPREPQVYTLPPSRDELTKNQVSLTCLVKGFYPSDIA |
| | VEWESNGQPENNYKTTPLVLDSDGSFFLYSKLTVDKSRWQQG |
| | NVFSCSVMHEGLHNHTTQKSLSLSPGK |
| | (SEQ ID NO: 64) |

In some embodiments, provided herein are IgG1 antibodies having a heavy chain constant region (CH) having at least 85% sequence identity to an amino acid sequence selected from the group consisting of SEQ ID NOs: 45-64. In some embodiments, the IgG1 antibodies provided herein have a CH having at least 85%, at least 90%, at least 95%, at least 98%, or at least 99% sequence identity to SEQ ID NO: 45. In some embodiments, the IgG1 antibodies provided herein have a CH having at least 85%, at least 90%, at least 95%, at least 98%, or at least 99% sequence identity to SEQ ID NO: 46. In some embodiments, the IgG1 antibodies provided herein have a CH having at least 85%, at least 90%, at least 95%, at least 98%, or at least 99% sequence identity to SEQ ID NO: 47. In some embodiments, the IgG1 antibodies provided herein have a CH having at least 85%, at least 90%, at least 95%, at least 98%, or at least 99% sequence identity to SEQ ID NO: 48. In some embodiments, the IgG1 antibodies provided herein have a CH having at least 85%, at least 90%, at least 95%, at least 98%, or at least 99% sequence identity to SEQ ID NO: 49. In some embodiments, the IgG1 antibodies provided herein have a CH having at least 85%, at least 90%, at least 95%, at least 98%, or at least 99% sequence identity to SEQ ID NO: 50. In some embodiments, the IgG1 antibodies provided herein have a CH having at least 85%, at least 90%, at least 95%, at least 98%, or at least 99% sequence identity to SEQ ID NO: 51. In some embodiments, the IgG1 antibodies provided herein have a CH having at least 85%, at least 90%, at least 95%, at least 98%, or at least 99% sequence identity to SEQ ID NO: 52. In some embodiments, the IgG1 antibodies provided herein have a CH having at least 85%, at least 90%, at least 95%, at least 98%, or at least 99% sequence identity to SEQ ID NO: 53. In some embodiments, the IgG1 antibodies provided herein have a CH having at least 85%, at least 90%, at least 95%, at least 98%, or at least 99% sequence identity to SEQ ID NO: 54. In some embodiments, the IgG1 antibodies provided herein have a CH having at least 85%, at least 90%, at least 95%, at least 98%, or at least 99% sequence identity to SEQ ID NO: 55. In some embodiments, the IgG1 antibodies provided herein have a CH having at least 85%, at least 90%, at least 95%, at least 98%, or at least 99% sequence identity to SEQ ID NO: 56. In some embodiments, the IgG1 antibodies provided herein have a CH having at least 85%, at least 90%, at least 95%, at least 98%, or at least 99% sequence identity to SEQ ID NO: 57. In some embodiments, the IgG1 antibodies provided herein have a CH having at least 85%, at least 90%, at least 95%, at least 98%, or at least 99% sequence identity to SEQ ID NO: 58. In some embodiments, the IgG1 antibodies provided herein have a CH having at least 85%, at least 90%, at least 95%, at least 98%, or at least 99% sequence identity to SEQ ID NO: 59. In some embodiments, the IgG1 antibodies provided herein have a CH having at least 85%, at least 90%, at least 95%, at least 98%, or at least 99% sequence identity to SEQ ID NO: 60. In some embodiments, the IgG1 antibodies provided herein have a CH having at least 85%, at least 90%, at least 95%, at least 98%, or at least 99% sequence identity to SEQ ID NO: 61. In some embodiments, the IgG1 antibodies provided herein have a CH having at least 85%, at least 90%, at least 95%, at least 98%, or at least 99% sequence identity to SEQ ID NO: 62. In some embodiments, the IgG1 antibodies provided herein have a CH having at least 85%, at least 90%, at least 95%, at least 98%, or at least 99% sequence identity to SEQ ID NO: 63. In some embodiments, the IgG1 antibodies provided herein have a CH having at least 85%, at least 90%, at least 95%, at least 98%, or at least 99% sequence identity to SEQ ID NO: 64.

In some embodiments, the provided antibody or antigen-binding fragment includes a CH having the amino acid sequence of SEQ ID NO: 55. In some embodiments (e.g., embodiments in which the antibody or antigen-binding fragment is the humanized antibody hu-cmAb12), the antibody or antigen-binding fragment includes a CH having the amino acid sequence of SEQ ID NO: 55, a VH having the amino acid sequence of SEQ ID NO: 26, and a VL having the amino acid sequence of SEQ ID NO: 19. In some embodiments, the antibody or antigen-binding fragment includes a CH having the amino acid sequence of SEQ ID NO: 55, a CL having the amino acid sequence of SEQ ID NO: 30, a VH having the amino acid sequence of SEQ ID NO: 26, and a VL having the amino acid sequence of SEQ ID NO: 19. Selected properties of humanized antibody hu-cmAB12 are demonstrated in Examples 8-17.

In some embodiments, variants can include addition of amino acid residues at the amino- and/or carboxyl-terminal end of the antibody or polypeptide. The length of additional amino acids residues can range from one residue to a hundred or more residues. In some embodiments, a variant comprises an N-terminal methionyl residue. In some embodiments, the variant comprises an additional polypeptide/protein (e.g., Fc region) to create a fusion protein. In some embodiments, a variant is engineered to be detectable and can comprise a detectable label and/or protein (e.g., a fluorescent tag or an enzyme).

The variant antibodies or antigen-binding fragments described herein can be generated using methods known in the art, including but not limited to, site-directed mutagenesis, alanine scanning mutagenesis, and PCR mutagenesis. Methods for mutagenesis and nucleotide sequence alterations are well known in the art. See, for example, Walker and Gaastra, eds. (1983) TECHNIQUES IN MOLECULAR BIOLOGY (MacMillan Publishing Company, New York); Kunkel, Proc. Natl. Acad. Sci. USA 82:488-492 (1985); Kunkel et al., Methods Enzymol. 54:367-382 (1987); Sambrook et al. (1989) MOLECULAR CLONING: A LABORATORY MANUAL (Cold Spring Harbor, N.Y.); U.S. Pat. No. 4,873,192; and the references cited therein; herein incorporated by reference. Guidance as to appropriate amino acid substitutions that do not affect biological activity of the polypeptide of interest can be found in the model of Dayhoff et al. (1978) in Atlas of Protein Sequence and Structure (Natl. Biomed. Res. Found., Washington, D.C.), pp. 345-352, herein incorporated by reference in its entirety. The model of Dayhoff et al. uses the Point Accepted Mutation (PAM) amino acid similarity matrix (PAM 250 matrix) to determine suitable conservative amino acid substitutions. Conservative substitutions, such as exchanging one amino acid with another having similar properties, can be beneficial. Examples of conservative amino acid substitutions as taught by the PAM 250 matrix of the Dayhoff et al. model include, but are not limited to, Gly→Ala, Val→Ile→Leu, Asp→Glu, Lys→Arg, Asn→Gln, and Phe→Trp→Tyr.

In constructing variants of an anti-ILT7 binding molecule, e.g., an antibody or antigen-binding fragment, variant, or derivative thereof, modifications are made such that variants continue to possess the desired properties, e.g., being capable of specifically binding to an ILT7, and in certain embodiments being able to inhibit IFN-alpha release, and/or to deplete pDCs in vivo. Obviously, any mutations made in the DNA encoding the variant polypeptide must not place the sequence out of reading frame. In some embodiment, mutations made in the DNA do not create complementary regions that could produce secondary mRNA structure.

In some embodiments, a variant of an anti-ILT7 antibody or antigen-binding fragment disclosed herein can retain the ability to bind ILT7 to a similar extent, the same extent, or to a higher extent, as the parent antibody or antigen-binding fragment. In some embodiments, the variant can be at least about 80%, about 85%, about 90%, about 91%, about 92%, about 93%, about 94%, about 95%, about 96%, about 97%, about 98%, about 99% or more identical in amino acid sequence to the parent antibody or antigen-binding fragment. In certain embodiments, a variant of an anti-ILT7 antibody or antigen-binding fragment comprises the amino acid sequence of the parent anti-ILT7 antibody or antigen-binding fragment with one or more conservative amino acid substitution. Conservative amino acid substitutions are known in the art and include amino acid substitutions in which one amino acid having certain physical and/or chemical properties is exchanged for another amino acid that has the same or similar chemical or physical properties.

In some embodiments, a variant of an anti-ILT7 antibody or antigen-binding fragment comprises the amino acid sequence of the parent antibody or antigen-binding fragment with one or more non-conservative amino acid substitutions. In some embodiments, a variant of an anti-ILT7 antibody or antigen-binding fragment comprises the amino acid sequence of the parent binding antibody or antigen-binding fragment with one or more non-conservative amino acid substitution, wherein the one or more non-conservative amino acid substitutions do not interfere with or inhibit one or more biological activities of the variant (e.g., ILT7 binding). In certain embodiments, the one or more conservative amino acid substitutions and/or the one or more non-conservative amino acid substitutions can enhance a biological activity of the variant, such that the biological activity of the functional variant is increased as compared to the parent antibody or antigen-binding fragment.

In some embodiments, the variant has 1, 2, 3, 4, or 5 amino acid substitutions in the CDRs (e.g., VH CDR1, VH CDR2, VH CDR3, VL CDR1, VL CDR2 and VL CDR3) of the binding moiety.

In some embodiments, anti-ILT7 antibodies or antigen-binding fragments described herein are chemically modified naturally or by intervention. In some embodiments, the anti-ILT7 antibodies or antigen-binding fragments have been chemically modified by glycosylation, acetylation, pegylation, phosphorylation, amidation, derivatization by known protecting/blocking groups, proteolytic cleavage, and/or linkage to a cellular ligand or other protein. Any of numerous chemical modifications can be carried out by known techniques. The anti-ILT7 antibodies or antigen-binding fragments can comprise one or more analogs of an amino acid (including, for example, unnatural amino acids), as well as other modifications known in the art.

In some embodiments, anti-ILT7 antibodies or antigen-binding fragments disclosed herein is linked to at least one agent to form an antibody conjugate. The conjugate can be, for example, an antibody conjugated to another protein, carbohydrate, lipid, steroids, immunosuppressors, or mixed moiety molecule(s). Such antibody conjugates include, but are not limited to, modifications that include linking the antibody to one or more polymers. For example, an antibody or antigen-binding fragment can be linked to one or more water-soluble polymers. Linkage to a water-soluble polymer reduces the likelihood that the antibody or antigen-binding fragment precipitates in an aqueous environment, such as a physiological environment. One skilled in the art can select a suitable water-soluble polymer based on considerations including, but not limited to, whether the polymer/antibody conjugate will be used in the treatment of a patient and, if so, the pharmacological profile of the antibody (e.g., half-life, dosage, activity, antigenicity, and/or other factors).

In order to increase the efficacy of antibody molecules as diagnostic or therapeutic agents, it is conventional to link or covalently bind or complex at least one desired molecule or moiety. Such a molecule or moiety can be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which can be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, an enzyme (e.g., that catalyzes a colorimetric or fluorometric or bioluminescent reaction), a substrate, a solid matrix, such as biotin. An antibody can comprise one, two, or more of any of these labels.

In some embodiments, anti-ILT7 antibodies or antigen-binding fragments described herein are chemically modified naturally or by intervention. In some embodiments, the anti-ILT7 antibodies or antigen-binding fragments have been chemically modified by glycosylation, acetylation, pegylation, phosphorylation, amidation, derivatization by known protecting/blocking groups, proteolytic cleavage, and/or linkage to a cellular ligand or other protein. Any of numerous chemical modifications can be carried out by known techniques. The anti-ILT7 antibodies or antigen-binding fragments can comprise one or more analogs of an amino acid (including, for example, unnatural amino acids), as well as other modifications known in the art.

Antibody conjugates can be used to deliver cytotoxic agents to target cells. Cytotoxic agents of this type can improve antibody-mediated cytotoxicity, and include such moieties as cytokines that directly or indirectly stimulate cell death, radioisotopes, chemotherapeutic drugs (including prodrugs), bacterial toxins (e.g., Pseudomonas exotoxin, diphtheria toxin, etc.), plant toxins (e.g., ricin, gelonin, etc.), chemical conjugates (e.g., maytansinoid toxins, calicheamicin, etc.), radioconjugates, enzyme conjugates (e.g., RNase conjugates, granzyme antibody-directed enzyme/prodrug therapy), and the like.

Antibody conjugates are also used as diagnostic agents. In some embodiments, an anti-ILT7 antibody or antigen-binding fragment described herein is conjugated to a detectable substance or molecule that allows the agent to be used for diagnosis and/or detection. A detectable substance can include, but is not limited to, enzymes; prosthetic groups (e.g., biotin and flavine(s)); fluorescent materials; bioluminescent materials, such as luciferase; radioactive materials; positron emitting metals; and magnetic metal ions positron emitting metals; and magnetic metal ions.

Antibody diagnostics generally fall within two classes, those for use in in vitro diagnostics, such as in a variety of immunoassays, and those for use in in vivo diagnostic protocols, generally known as “antibody-directed imaging.” Many appropriate imaging agents are known in the art, as are methods for their attachment to antibodies (see, e.g., U.S. Pat. Nos. 5,021,236, 4,938,948, and 4,472,509). The imaging moieties used can be paramagnetic ions, radioactive isotopes, fluorochromes, NMR-detectable substances, MR hyperpolarized molecules, targeted ultrasound bubbles, and X-ray imaging agents.

The paramagnetic ions contemplated for use as conjugates include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and bismuth (III). Alternative useful isotopes are those used for hyperpolarized MRI, such as carbon-13 and silica-29.

The radioactive isotopes contemplated for use in imaging and radiotherapy as conjugates or covalent incorporation include astatine-211, actinium-225, carbon-14, bismuth-212, chromium-51, chlorine-36, cobalt-57, cobalt-58, copper-64, copper-67, europium-152, fluorine-18, gallium-68, gallium-67, gold-198, hydrogen-3, iodine-123, iodine-125, iodine-131, indium-111, iron-52, iron-59, lead-212, lutetium-177, phosphorus-32, rhenium-186, rhenium-188, rubidium-82, rhodium-99, selenium-75, sulphur-35, samarium-153, strontium-92, strontium-89, thallium-201, thorium-227, technetium-94m, technetium-99m, yttrium-86, yttrium-90, zirconium-86, and/or zirconium-89. F-18, Zr-89 and Cu-64 often being preferred for PET imaging. Lu-177, At-211, and Yt-90 often being preferred for radiotherapy. Radioactively labeled monoclonal antibodies and antibody fragments of the present disclosure can be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure can be labeled with technetium-99m by ligand exchange process, for example, by reducing pertechnetate with stannous solution, chelating the reduced technetium onto a Sephadex column and applying the antibody to this column. Alternatively, direct labeling techniques can be used, e.g., by incubating pertechnetate, a reducing agent such as SNC12, a buffer solution such as sodium-potassium phthalate solution, and the antibody. Intermediary functional groups that incorporate chelators, which are often used to bind radioisotopes that exist as metallic ions to an antibody are diethylene-triamine-pentaacetic acid (DTPA), ethylene diamine-tetraacetic acid (EDTA), monomeric or dendrimeric 1,4,7,10-tetraazacyclododecane-1,4,7,10-tetraacetic acid (DOTA), 1,4,7-triazacyclononane-1,4,7-triacetic acid (NOTA), deferoxamine (DFO), or 1-hydroxy-2(1H)-pyridinone derivatives (e.g., 3,4,3-LI(1,2-HOPO) or HOPO).

The fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, cyanine (Cy3), Cy5,6-FAM, dansyl chloride, dichlorotriazinylamine fluorescein, fluorescein isothiocyanate (FITC), HEX, 6-JOE, Oregon Green 488, Oregon Green 500, Oregon Green 514, Pacific Blue, Phycoerythrin, REG, Rhodamine Green, Rhodamine Red, Renografin, ROX, TAMRA, TET, tetramethylrhodamine isothiocyanate (TRITC), Texas Red, and/or Umbelliferone.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include beta-galactosidase, acetylcholinesterase, urease, alkaline phosphatase, (horseradish) hydrogen peroxidase or glucose oxidase. Preferred secondary binding ligands are biotin and avidin and streptavidin compounds.

Several methods are known in the art for the attachment or conjugation of an antibody to its conjugate moiety. Some attachment methods involve the use of a metal chelate complex employing, for example, an organic chelating agent such a diethylene-triamine-pentaacetic acid anhydride (DTPA); ethylene-diamine-tetraacetic acid; monomeric or dendrimeric 1,4,7,10-tetraazacyclododecane-1,4,7,10-tetraacetic acid (DOTA); 1,4,7-triazacyclononane-1,4,7-triacetic acid (NOTA); DFO; HOPO; N-chloro-p-toluenesulfonamide; and/or tetrachloro-3a-6a-diphenylglycouril-3 attached to the antibody (U.S. Pat. Nos. 4,472,509 and 4,938,948). Monoclonal antibodies can also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl) propionate.

Another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction.

Molecules containing azido groups can also be used to form covalent bonds to proteins through reactive nitrene intermediates that are generated by low intensity ultraviolet light. In particular, 2- and 8-azido analogues of purine nucleotides have been used as site-directed photoprobes to identify nucleotide binding proteins in crude cell extracts. The 2- and 8-azido nucleotides have also been used to map nucleotide binding domains of purified proteins and can be used as antibody binding agents.

Derivatization of immunoglobulins by selectively introducing sulfhydryl groups in the Fe region of an immunoglobulin, using reaction conditions that do not alter the antibody combining site are also contemplated. Antibody conjugates produced according to this methodology are disclosed to exhibit improved longevity, specificity, and sensitivity (U.S. Pat. No. 5,196,066, incorporated herein by reference). Site-specific attachment of effector or reporter molecules, wherein the reporter or effector molecule is conjugated to a carbohydrate residue in the Fc region, have also been disclosed in the literature. This approach has been reported to produce diagnostically and therapeutically promising antibodies which are currently in clinical evaluation.

In some embodiments, the anti-ILT7 antibodies or antigen-binding fragments described herein are conjugated to a steroid or an immunosuppressor. In some embodiments, the antibody or antigen-binding fragment is conjugated to a steroid or an immunosuppressor to form an ADC (antibody-drug conjugate). In some embodiments, the anti-ILT7 antibodies or antigen-binding fragments described herein are conjugated to a steroid, which can be a corticosteroid. The corticosteroid can be, for example, dexamethasone, hydrocortisone, methylprednisolone, and prednisone. In some embodiments, the anti-ILT7 antibodies or antigen-binding fragments described herein are conjugated to an immunosuppressor, which can be an antimalarial (such as hydroxychloroquine, chloroquine), an antimetabolite (such as methotrexate, azathioprine, mercaptopurine), a calcineurin inhibitor (such as cyclosporin, tacrolimus), mycophenolic acid, mycophenolate mofetil, thalidomide, or acitretin.

In some embodiments, the anti-ILT7 antibodies or antigen-binding fragments described herein are conjugated to a cytotoxic agent or moiety. In some embodiments, the antibody or antigen-binding fragment is conjugated to a cytotoxic agent to form an ADC (antibody-drug conjugate). In some embodiments, antibody drug conjugates, or ADCs, are a class of highly potent biopharmaceutical drugs designed as a targeted therapy. ADCs are composed of an antibody (a whole mAb or an antibody fragment, such as a scFv) linked, via a stable chemical linker with labile bonds, to a biological active cytotoxic/anti-viral payload or drug. Antibody drug conjugates are examples of bioconjugates and immunoconjugates. By combining the unique targeting capabilities of monoclonal antibodies with cytotoxic drugs, ADCs allow sensitive discrimination between healthy and diseased tissue. This means that, in contrast to traditional systemic approaches, ADCs target and attack the diseased cell so that healthy cells are less severely affected.

In the development of ADC-based anti-tumor therapies, a warhead (e.g., a cell toxin or cytotoxin) is coupled to an antibody that specifically targets a certain cell marker (e.g., a protein that, ideally, is only to be found in or on diseased cells). Antibodies target these proteins in the body and attach themselves to the surface of the diseased cells. The biochemical reaction between the antibody and the target protein (antigen) triggers a signal in the targeted cell, which then absorbs or internalizes the antibody together with the cytotoxin. After the ADC is internalized, the cytotoxic drug is released and kills the cell or impairs cellular replication. In other cases, the linker is cleavable on the surface of the target cell or early endosomes, and as such, full internalization is not required. Due to this targeting, ideally the drug has lower side effects and gives a wider therapeutic window than other agents.

In some embodiments, the cytotoxic moiety of the ADCs having the anti-ILT7 antibodies or antigen-binding fragments described herein is a chemotherapeutic agent including, but not limited to, methotrexate, adriamycin/doxorubicin, melphalan, mitomycin C, chlorambucil, duocarmycin, daunorubicin, pyrrolobenzodiazepines (PBDs), or other intercalating agents. In some embodiments, the cytotoxic moiety is a microtubule inhibitor including, but not limited to, auristatins, maytansinoids (e.g., DM1 and DM4), and tubulysins. In some embodiments, the cytotoxic moiety is an enzymatically active toxin of bacterial, fungal, plant, or animal origin, or fragments thereof, including, but not limited to, diphtheria A chain, non-binding active fragments of diphtheria toxin, exotoxin A chain, ricin A chain, abrin A chain, modeccin A chain, alpha-sarcin, Aleurites fordii proteins, dianthin proteins, Phytolacca americana proteins (PAPI, PAPII, and PAP-S), Momordica charantia inhibitor, curcin, crotin, Saponaria officinalis inhibitor, gelonin, mitogellin, restrictocin, phenomycin, enomycin, and the trichothecenes. In some embodiments, an antibody or antigen-binding fragment is conjugated to one or more small molecule toxins, such as calicheamicins, maytansinoids, trichothecenes, and CC1065.

A stable link between the antibody and cytotoxic agent is a crucial aspect of an ADC. Linkers are based on chemical motifs including disulfides, hydrazones or peptides (cleavable), or thioethers (non-cleavable), and control the distribution and delivery of the cytotoxic agent to the target cell. Cleavable and non-cleavable types of linkers have been proven to be safe in preclinical and clinical trials. The availability of better and more stable linkers has changed the function of the chemical bond. The type of linker, cleavable or non-cleavable, lends specific properties to the cytotoxic (e.g., anti-cancer) drug. For example, a non-cleavable linker keeps the drug within the cell. As a result, the entire antibody, linker, and cytotoxic agent enter the targeted cell where the antibody is degraded to the level of amino acids. The resulting complex—amino acid, linker, and cytotoxic agent—now becomes the active drug. In contrast, cleavable linkers are catalyzed by enzymes in or on the host cell, thereby releasing the cytotoxic agent. Commonly used mechanisms for linker cleavage are protease sensitivity, pH sensitivity, and glutathione sensitivity. Another type of cleavable linker adds an extra molecule between the cytotoxic drug and the cleavage site. This linker technology allows researchers to create ADCs with more flexibility without changing cleavage kinetics. A new method of peptide cleavage based on Edman degradation has also been developed. Future direction in the development of ADCs also includes the development of site-specific conjugation (TDCs) to further improve stability and therapeutic index and a-emitting immunoconjugates and antibody-conjugated nanoparticles.

An anti-ILT7 antibody or antigen-binding fragment described herein can be attached to a solid support. Such solid supports include, but are not limited to, glass, cellulose, polyacrylamide, nylon, polystyrene, polyvinyl chloride, or polypropylene. In some embodiments, an immobilized anti-ILT7 antibody or antigen-binding fragment is used in an immunoassay. In some embodiments, an immobilized anti-ILT7 antibody or antigen-binding fragment is used in purification of the target antigen (e.g., human ILT7).

### D. Polynucleotides and Vectors

Also provided herein are polynucleotides that encode a polypeptide (e.g., an anti-ILT7 antibody or antigen-binding fragment) described herein. The term “polynucleotide that encodes a polypeptide” encompasses a polynucleotide which includes only coding sequences for the polypeptide as well as a polynucleotide which includes additional coding and/or non-coding sequences. The polynucleotides of the disclosure can be in the form of RNA or in the form of DNA. DNA can be cDNA, genomic DNA, or synthetic DNA, and can be double-stranded or single-stranded. Single stranded DNA can be the coding strand or non-coding (anti-sense) strand. The polynucleotides of the disclosure can be mRNA.

Expressly contemplated herein are polynucleotides that encode any anti-ILT7 antibody or antigen-binding fragment disclosed herein. For illustrative purposes, in some embodiments, the polynucleotides provided herein encode an anti-ILT7 antibody or antigen-binding fragment comprising (1) as defined by Kabat, (a) a light chain variable region (VL) comprising VL CDR1, VL CDR2, and VL CDR3 having the amino acid sequences of SEQ ID NOs: 11, 12, and 13, respectively; or a variant thereof having up to about 5 amino acid substitutions, additions, and/or deletions in the VL CDRs; and/or (b) a heavy chain variable region (VH) comprising VH CDR1, VH CDR2, and VH CDR3 having the amino acid sequences of SEQ ID NOs: 14, 15, and 16, respectively; or a variant thereof having up to about 5 amino acid substitutions, additions, and/or deletions in the VH CDRs; or (2) as defined by Chothia, (a) a VL comprising VL CDR1, VL CDR2, and VL CDR3 having the amino acid sequences of SEQ ID NOs: 11, 12, and 13, respectively; or a variant thereof having up to about 5 amino acid substitutions, additions, and/or deletions in the VL CDRs; and/or (b) a VH comprising VH CDR1, VH CDR2, and VH CDR3 having the amino acid sequences of SEQ ID NOs: 17, 18, and 16, respectively; or a variant thereof having up to about 5 amino acid substitutions, additions, and/or deletions in the VH CDRs.

In some embodiments, the polynucleotides provided herein encode an anti-ILT7 antibody or antigen-binding fragment comprising (a) a VL having at least 85%, at least 90%, at least 95%, at least 98%, or 100% sequence identity to the amino acid sequence of SEQ ID NO: 9; and/or (b) a VH having at least 85%, at least 90%, at least 95%, at least 98%, or 100% sequence identity to the amino acid sequence of SEQ ID NO: 10. The polynucleotides can be in the form of DNA. The polynucleotides can be in the form of mRNA.

In some embodiments, the polynucleotides provided herein encode an anti-ILT7 antibody or antigen-binding fragment disclosed herein comprising a VL and a VH, wherein the VL comprises VL CDR1, CDR2 and CDR3 and the VH comprises VH CDR1, CDR2 and CDR3, and wherein the VL CDR1, VL CDR2, VL CDR3, VH CDR1, VH CDR2 and VH CDR3 have the amino acid sequences of (1) SEQ ID NOs: 11, 12, 13, 14, 15 and 16, respectively; or (2) SEQ ID NOs: 11, 12, 13, 17, 18 and 16, respectively; or a variant thereof having up to about 5 amino acid substitutions, additions, and/or deletions in the CDRs. The polynucleotides can be in the form of DNA. The polynucleotides can be in the form of mRNA.

In some embodiments, the polynucleotides provided herein encode an anti-ILT7 antibody or antigen-binding fragment disclosed herein comprising a VL and a VH, wherein the VL and VH have the amino acid sequences of (1) SEQ ID NOs: 9 and 10, respectively; (2) SEQ ID NOs: 19 and 23, respectively; (3) SEQ ID NOs: 19 and 24, respectively; (4) SEQ ID NOs: 19 and 25, respectively; (5) SEQ ID NOs: 19 and 26, respectively; (6) SEQ ID NOs: 19 and 27, respectively; (7) SEQ ID NOs: 19 and 28, respectively; (8) SEQ ID NOs: 20 and 23, respectively; (9) SEQ ID NOs: 20 and 24, respectively; (10) SEQ ID NOs: 20 and 25, respectively; (11) SEQ ID NOs: 20 and 26, respectively; (12) SEQ ID NOs: 20 and 27, respectively; (13) SEQ ID NOs: 20 and 28, respectively; (14) SEQ ID NOs: 21 and 23, respectively; (15) SEQ ID NOs: 21 and 24, respectively; (16) SEQ ID NOs: 21 and 25, respectively; (17) SEQ ID NOs: 21 and 26, respectively; (18) SEQ ID NOs: 21 and 27, respectively; (19) SEQ ID NOs: 21 and 28, respectively; (20) SEQ ID NOs: 22 and 23, respectively; (21) SEQ ID NOs: 22 and 24, respectively; (22) SEQ ID NOs: 22 and 25, respectively; (23) SEQ ID NOs: 22 and 26, respectively; (24) SEQ ID NOs: 22 and 27, respectively; or (25) SEQ ID NOs: 22 and 28, respectively. The polynucleotides can be in the form of DNA. The polynucleotides can be in the form of mRNA.

In some embodiments, the VL and VH are connected by a linker. The linker can be a flexible linker or a rigid linker. In some embodiments, the linker has the amino acid sequence of (GGGGS)n, n=1, 2, 3, 4, or 5 (SEQ ID NO: 35). In some embodiments, the linker has the amino acid sequence of (EAAAK)n, n=1, 2, 3, 4, or 5 (SEQ ID NO: 36). In some embodiments, the linker has the amino acid sequence of (PA)nP, n=1, 2, 3, 4, or 5 (SEQ ID NO: 37).

The present disclosure also provides variants of the polynucleotides described herein, wherein the variants encode, for example, fragments, analogs, and/or derivatives of an anti-ILT7 antibody or antigen-binding fragment disclosed herein. In some embodiments, the present disclosure provides a polynucleotide having a nucleotide sequence at least about 80% identical, at least about 85% identical, at least about 90% identical, at least about 95% identical, at least about 96% identical, at least about 97% identical, at least about 98% identical, or at least about 99% identical to a polynucleotide sequence encoding an anti-ILT7 antibody or antigen-binding fragment described herein. In some embodiments, the present disclosure provides a polynucleotide having a nucleotide sequence at least about 80% identical, at least about 85% identical, at least about 90% identical, at least about 95% identical, at least about 96% identical, at least about 97% identical, at least about 98% identical, or at least about 99% identical to a polynucleotide sequence encoding an anti-ILT7 antibody or antigen-binding fragment described herein.

As used herein, the phrase “a polynucleotide having a nucleotide sequence at least about 95% identical to a polynucleotide sequence” means that the nucleotide sequence of the polynucleotide is identical to a reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but do not alter the properties or activities of the encoded polypeptide. In some embodiments, a polynucleotide variant comprises silent substitutions that result in no change to the amino acid sequence of the polypeptide (due to the degeneracy of the genetic code). Polynucleotide variants can be produced for a variety of reasons, for example, to optimize codon expression for a particular host (e.g., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In some embodiments, a polynucleotide comprises the coding sequence for a polypeptide (e.g., an antibody) fused in the same reading frame to a polynucleotide which aids in expression and secretion of a polypeptide from a host cell (e.g., a leader sequence which functions as a secretory sequence for controlling transport of a polypeptide). The polypeptide can have the leader sequence cleaved by the host cell to form a “mature” form of the polypeptide.

In some embodiments, a polynucleotide comprises the coding sequence for a polypeptide (e.g., an antibody) fused in the same reading frame to a marker or tag sequence. For example, in some embodiments, a marker sequence is a hexa-histidine tag (HIS-tag) (SEQ ID NO: 65) that allows for efficient purification of the polypeptide fused to the marker. In some embodiments, a marker sequence is a hemagglutinin (HA) tag derived from the influenza hemagglutinin protein when a mammalian host (e.g., COS-7 cells) is used. In some embodiments, the marker sequence is a FLAG™ tag. In some embodiments, a marker is used in conjunction with other markers or tags.

In some embodiments, a polynucleotide is isolated. In some embodiments, a polynucleotide is substantially pure.

Vectors and cells comprising the polynucleotides described herein are also provided. In some embodiments, provided herein are vectors comprising a polynucleotide provided herein. The vectors can be expression vectors. In some embodiments, vectors provided herein comprise a polynucleotide encoding an anti-ILT7 antibody or antigen-binding fragment described herein. In some embodiments, vectors provided herein comprise a polynucleotide encoding a polypeptide that is part of an anti-ILT7 antibody or antigen-binding fragment described herein.

In some embodiments, provided herein are recombinant expression vectors, which can be used to amplify and express a polynucleotide encoding an anti-ILT7 antibody or antigen-binding fragment described herein. For example, a recombinant expression vector can be a replicable DNA construct that includes synthetic or cDNA-derived DNA fragments encoding a polypeptide chain of an anti-ILT7 antibody, operatively linked to suitable transcriptional and/or translational regulatory elements derived from mammalian, microbial, viral or insect genes. In some embodiments, a viral vector is used. DNA regions are “operatively linked” when they are functionally related to each other. For example, a promoter is operatively linked to a coding sequence if it controls the transcription of the sequence; or a ribosome binding site is operatively linked to a coding sequence if it is positioned so as to permit translation. In some embodiments, structural elements intended for use in certain expression systems include a leader sequence enabling extracellular secretion of translated protein by a host cell. In some embodiments, in situations where recombinant protein is expressed without a leader or transport sequence, a polypeptide can include an N-terminal methionine residue.

A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages. In some embodiments, an anti-ILT7 antibody or antigen-binding fragment described herein is expressed from one or more vectors.

Provided herein are host cells comprising vectors described herein. In some embodiments, the host cells are used for recombination expression of the anti-ILT7 antibodies descried herein. The host cells can include prokaryotes, yeast cells, insect cells, or higher eukaryotic cells under the control of appropriate promoters. Appropriate cloning and expression vectors for use with bacterial, fungal, yeast, and mammalian cellular hosts, as well as methods of protein production, including antibody production are well-known in the art.

Examples of suitable mammalian host cells include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

The present disclosure also provides host cells comprising the polypeptides described herein, polynucleotides encoding polypeptides described herein, or vectors comprising such polynucleotides. In some embodiments, provided herein are host cells comprising a vector comprising a polynucleotide disclosed herein. In some embodiments, host cells provided herein comprise a vector comprising a polynucleotide encoding an anti-ILT7 antibody or antigen-binding fragment described herein. In some embodiments, host cells provided herein comprise a vector comprising a polynucleotide encoding a polypeptide that is part of an anti-ILT7 antibody or antigen-binding fragment described herein. In some embodiments, host cells provided herein comprise a polynucleotide encoding an anti-ILT7 antibody or antigen-binding fragment described herein. In some embodiments, the cells produce the anti-ILT7 antibodies or antigen-binding fragments described herein.

### E. Methods of Manufacture

Provided herein are also methods of manufacturing anti-ILT7 antibodies and antigen-binding fragments thereof that include but are not limited to monoclonal antibodies, polyclonal antibodies, synthetic antibodies, human antibodies, humanized antibodies, and antigen-binding fragments thereof.

Methods of antibody production are well-known in the art. See for example, in Harlow et al., Antibodies: A Laboratory Manual, (Cold Spring Harbor Laboratory Press, 2nd ed. 1988); Hammerling et al., in: MONOCLONAL ANTIBODIES AND T-CELL HYBRIDOMAS 563 681 (Elsevier, N.Y., 1981), each of which is incorporated herein by reference in its entirety. In some embodiments, monoclonal antibodies are prepared using hybridoma methods known to one of skill in the art. For example, using a hybridoma method, a mouse, rat, rabbit, hamster, or other appropriate host animal, is immunized as described above. In some embodiments, lymphocytes are immunized in vitro. In some embodiments, the immunizing antigen is a human protein or a fragment thereof. In some embodiments, the immunizing antigen is a human protein or a fragment thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed to a chosen antigen can be identified by a variety of methods including, but not limited to, immunoprecipitation, immunoblotting, and in vitro binding assays (e.g., flow cytometry, FACS, ELISA, SPR (e.g., Biacore), and radioimmunoassay). Once hybridoma cells that produce antibodies of the desired specificity, affinity, and/or activity are identified, the clones may be subcloned by limiting dilution or other techniques. The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In some embodiments, monoclonal antibodies are made using recombinant DNA techniques as known to one skilled in the art. For example, the polynucleotides encoding an antibody are isolated from mature B-cells or hybridoma cells, such as by RT-PCR using oligonucleotide primers that specifically amplify the genes encoding the heavy and light chains of the antibody, and their sequence is determined using standard techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins.

Polynucleotides of the antibodies or antigen-binding fragments provided herein can be prepared, manipulated, and/or expressed using any of the well-established techniques known and available in the art. In some embodiments, polynucleotides of the antibodies or antigen-binding fragments provided herein can be prepared recombinantly. Many vectors can be used. Examples of vectors are plasmid, autonomously replicating sequences, and transposable elements. Exemplary transposon systems such as Sleeping Beauty and PiggyBac can be used, which can be stably integrated into the genome (e.g., Ivics et al., Cell, 91 (4): 501-510 (1997); Cadiñanos et al., (2007) Nucleic Acids Research. 35 (12): e87). Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pCl-neo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells.

In some embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally. The vector is engineered to harbor the sequence coding for the origin of DNA replication or “ori” from a lymphotropic herpes virus or a gamma herpesvirus, an adenovirus, SV40, a bovine papilloma virus, or a yeast, specifically a replication origin of a lymphotropic herpes virus or a gamma herpesvirus corresponding to oriP of EBV. In some embodiments, the lymphotropic herpes virus is Epstein Barr virus (EBV), Kaposi's sarcoma herpes virus (KSHV), Herpes virus saimiri (HS), or Marek's disease virus (MDV). Epstein Barr virus (EBV) and Kaposi's sarcoma herpes virus (KSHV) are also examples of a gamma herpesvirus. Typically, the host cell comprises the viral replication transactivator protein that activates the replication.

“Expression control sequences,” “control elements,” or “regulatory sequences” present in an expression vector are those non-translated regions of the vector—origin of replication, selection cassettes, promoters, enhancers, translation initiation signals (Shine Dalgarno sequence or Kozak sequence) introns, a polyadenylation sequence, 5′ and 3′ untranslated regions-which interact with host cellular proteins to carry out transcription and translation. Such elements can vary in their strength and specificity. Depending on the vector system and host utilized, any number of suitable transcription and translation elements, including ubiquitous promoters and inducible promoters, can be used.

Illustrative ubiquitous expression control sequences that can be used in present disclosure include, but are not limited to, a cytomegalovirus (CMV) immediate early promoter, a viral simian virus 40 (SV40) promoter (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF 1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70-kDa protein 5 (HSPA5), heat shock protein 90-kDa beta, member 1 (HSP90B1), heat shock protein 70-kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, and a β-actin promoter.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc. The anti-ILT7 antibodies or antigen-binding fragments described herein can be produced by any method known in the art, including chemical synthesis and recombinant expression techniques. The practice of the invention employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art.

The polypeptides described herein can be prepared using a wide variety of techniques known in the art including the use of hybridoma and recombinant technologies, or a combination thereof. In some embodiments, a recombinant expression vector is used to express a polynucleotide encoding a polypeptide described herein. For example, a recombinant expression vector can be a replicable DNA construct that includes synthetic or cDNA-derived DNA fragments encoding a polypeptide operatively linked to suitable transcriptional and/or translational regulatory elements derived from mammalian, microbial, viral or insect genes. In some embodiments, coding sequences of polypeptides disclosed herein can be ligated into such expression vectors for their expression in mammalian cells. In some embodiments, a viral vector is used. DNA regions are “operatively linked” when they are functionally related to each other. For example, a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In some embodiments, in situations where recombinant protein is expressed without a leader or transport sequence, a polypeptide can include an N-terminal methionine residue.

A wide variety of expression host/vector combinations can be employed. Suitable host cells for expression include prokaryotes, yeast cells, insect cells, or higher eukaryotic cells under the control of appropriate promoters. Appropriate cloning and expression vectors for use with bacterial, fungal, yeast, and mammalian cellular hosts, as well as methods of protein production, including antibody production are well-known in the art. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

Useful expression vectors for eukaryotic hosts include, for example, vectors comprising expression control sequences from SV40, bovine papilloma virus, adenovirus, and cytomegalovirus. Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

To make anti-ILT7 antibodies and antigen-binding fragments that are afucosylated, host cells that (1) overexpress N-acetylglucosaminyltransferase III (GnTIII), (2) lack a-1,6-fucosyltransferase (FUT8), or (3) have a low fucose content, or any combination of (1)-(3) can be used. In some embodiments, used herein are host cells that overexpresses N-acetylglucosaminyltransferase III (GnTIII). In some embodiments, used herein are host cells that lack a 1,6-fucosyltransferase (FUT8). In some embodiments, used herein are host cells having a low fucose content. In some embodiments, CHO host cells are used.

Peptides can be synthesized, in whole or in part, using chemical methods (see, e.g., Caruthers (1980). Nucleic Acids Res. Symp. Ser. 215; Horn (1980); and Banga, A. K., THERAPEUTIC PEPTIDES AND PROTEINS, FORMULATION, PROCESSING AND DELIVERY SYSTEMS (1995) Technomic Publishing Co., Lancaster, PA). Peptide synthesis can be performed using various solid phase techniques (see, e.g., Roberge, Science 269:202 (1995); Merrifield, Methods. Enzymol. 289:3 (1997)) and automated synthesis may be achieved, e.g., using the ABI 431A Peptide Synthesizer (Perkin Elmer) in accordance with the manufacturer's instructions. Peptides can also be synthesized using combinatorial methodologies. Synthetic residues and polypeptides can be synthesized using a variety of procedures and methodologies known in the art (see, e.g., ORGANIC SYNTHESES COLLECTIVE VOLUMES, Gilman, et al. (Eds) John Wiley & Sons, Inc., NY). Modified peptides can be produced by chemical modification methods (see, for example, Belousov, Nucleic Acids Res. 25:3440 (1997); Frenkel, Free Radic. Biol. Med. 19:373 (1995); and Blommers, Biochemistry 33:7886 (1994)). Peptide sequence variations, derivatives, substitutions, and modifications can also be made using methods such as oligonucleotide-mediated (site-directed) mutagenesis, alanine scanning, and PCR based mutagenesis. Site-directed mutagenesis (Carter et al., Nucl. Acids Res., 13:4331 (1986); Zoller et al., Nucl. Acids Res. 10:6487 (1987)), cassette mutagenesis (Wells et al., Gene 34:315 (1985)), restriction selection mutagenesis (Wells et al., Philos. Trans. R. Soc. London SerA 317:415 (1986)) and other techniques can be performed on cloned DNA to produce invention peptide sequences, variants, fusions and chimeras, and variations, derivatives, substitutions, and modifications thereof.

For in vivo use of antibodies in humans, it may be preferable to use human antibodies. Completely human antibodies are particularly desirable for therapeutic treatment of human subjects. Human antibodies can be made by a variety of methods known in the art including phage display methods using antibody libraries derived from human immunoglobulin sequences, including improvements to these techniques. See, also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety. A human antibody can also be an antibody wherein the heavy and light chains are encoded by a nucleotide sequence derived from one or more sources of human DNA.

In some embodiments, an anti-ILT7 antibody or antigen-binding fragment is a human antibody or antigen-binding fragment. Human antibodies can be prepared using various techniques known in the art. In some embodiments, human antibodies are generated from immortalized human B lymphocytes immunized in vitro. In some embodiments, human antibodies are generated from lymphocytes isolated from an immunized individual. In any case, cells that produce an antibody directed against a target antigen can be generated and isolated. In some embodiments, a human antibody is selected from a phage library, where that phage library expresses human antibodies. Alternatively, phage display technology can be used to produce human antibodies and antibody fragments in vitro, from immunoglobulin variable region gene repertoires from unimmunized donors. Techniques for the generation and use of antibody phage libraries are well-known in the art. Once antibodies are identified, affinity maturation strategies known in the art, including but not limited to, chain shuffling and site-directed mutagenesis, can be employed to generate higher affinity human antibodies. In some embodiments, human antibodies are produced in transgenic mice that contain human immunoglobulin loci. Upon immunization these mice can produce the full repertoire of human antibodies in the absence of endogenous immunoglobulin production.

Human antibodies can also be produced using transgenic mice which are incapable of expressing functional endogenous immunoglobulins, but which can express human immunoglobulin genes. For example, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. For example, it has been described that the homozygous deletion of the antibody heavy chain joining region (JH) gene in chimeric and germ-line mutant mice results in complete inhibition of endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. For example, anti-ILT7 antibodies directed against the human ILT7 antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies, including, but not limited to, IgG1 (gamma 1) and IgG3. For an overview of this technology for producing human antibodies, see, Lonberg and Huszar (Int. Rev. Immunol., 13:65-93 (1995)). For a detailed discussion of this technology for producing human antibodies and human monoclonal antibodies and protocols for producing such antibodies, see, e.g., PCT Publication Nos. WO 98/24893, WO 96/34096, and WO 96/33735; and U.S. Pat. Nos. 5,413,923; 5,625,126; 5,633,425; 5,569,825; 5,661,016; 5,545,806; 5,814,318; and 5,939,598, each of which is incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above. For a specific discussion of transfer of a human germ-line immunoglobulin gene array in germ-line mutant mice that will result in the production of human antibodies upon antigen challenge see, e.g., Jakobovits et al., Proc. Natl. Acad. Sci. USA, 90:2551 (1993); Jakobovits et al., Nature, 362:255-258 (1993); Bruggermann et al., Year in Immunol., 7:33 (1993); and Duchosal et al., Nature, 355:258 (1992).

Human antibodies can also be derived from phage-display libraries (Hoogenboom et al., J. Mol. Biol., 227:381 (1991); Marks et al., J. Mol. Biol., 222:581-597 (1991); Vaughan et al., Nature Biotech., 14:309 (1996)). Phage display technology (McCafferty et al., Nature, 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson and Chiswell, Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of unimmunized mice. A repertoire of V genes from unimmunized human donors can be constructed and antibodies to a diverse array of antigens (including self-antigens) can be isolated essentially following the techniques described by Marks et al., J. Mol. Biol., 222:581-597 (1991), or Griffith et al., EMBO J., 12:725-734 (1993). See, also, U.S. Pat. Nos. 5,565,332 and 5,573,905, each of which is incorporated herein by reference in its entirety.

Human antibodies can also be generated by in vitro activated B cells (see, U.S. Pat. Nos. 5,567,610 and 5,229,275, each of which is incorporated herein by reference in its entirety). Human antibodies can also be generated in vitro using hybridoma techniques such as, but not limited to, that described by Roder et al. (Methods Enzymol., 121:140-167 (1986)).

Alternatively, in some embodiments, a non-human antibody is humanized, where specific sequences or regions of the antibody are modified to increase similarity to an antibody naturally produced in a human. In some embodiment, the antigen binding domain portion is humanized. Various methods for generating humanized antibodies are known in the art. Methods are known in the art for achieving high affinity binding with humanized antibodies. A non-limiting example of such a method is hypermutation of the variable region and selection of the cells expressing such high affinity antibodies (affinity maturation). In addition to the use of display libraries, the specified antigen (e.g., recombinant ILT7 or an epitope thereof) can be used to immunize a non-human animal, e.g., a rodent. In certain embodiments, rodent antigen-binding fragments (e.g., mouse antigen-binding fragments) can be generated and isolated using methods known in the art and/or disclosed herein. In some embodiments, a mouse can be immunized with an antigen (e.g., recombinant ILT7 or an epitope thereof).

A humanized antibody can be produced using techniques including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions can be substituted with the corresponding residue from the CDR donor antibody to alter, preferably improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody has one or more amino acid residues introduced into it from a source which is nonhuman. These nonhuman amino acid residues are often referred to as “import” residues, which are typically taken from an “import” variable domain. Thus, humanized antibodies comprise one or more CDRs from nonhuman immunoglobulin molecules and framework regions from human. Humanization of antibodies is well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized chimeric antibodies, substantially less than an intact human variable domain has been substituted by the corresponding sequence from a nonhuman species. In practice, humanized antibodies are typically human antibodies in which some CDR residues and possibly some FR residues are substituted by residues from analogous sites in rodent antibodies. Humanization of antibodies can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993), the contents of which are incorporated herein by reference herein in their entirety).

Antibodies can be humanized with retention of high affinity for the target antigen and other favorable biological properties. For example, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind the target antigen. In this way, FR residues can be selected and combined from the recipient and import sequences so that the desired antibody characteristic, such as increased affinity for the target antigen, is achieved. In general, the CDR residues are directly and most substantially involved in influencing antigen binding.

A humanized antibody retains a similar antigenic specificity as the original antibody, for example, the ability to bind human ILT7 antigen. However, using certain methods of humanization, the affinity and/or specificity of binding of the antibody for a particular antigen can be increased using methods of “directed evolution,” as described by Wu et al., J. Mol. Biol., 294:151 (1999), the contents of which are incorporated herein by reference herein in their entirety.

Anti-ILT7 antibodies or antigen-binding fragments described herein can be tested for binding to human ILT7 by, for example, standard ELISA. Briefly, microtiter plates are coated with purified ILT7, and then blocked with bovine serum albumin. Dilutions of antibody (e.g., dilutions of plasma from ILT7-immunized mice) are added to each well and incubated. The plates are washed and incubated with secondary reagent (e.g., for human antibodies, a goat-anti-human IgG Fc-specific polyclonal reagent) conjugated to horseradish peroxidase (HRP). After washing, the plates can be developed and analyzed by a spectrophotometer. Sera from immunized mice can then be further screened by flow cytometry for binding to a cell line expressing human ILT7, but not to a control cell line that does not express ILT7. Briefly, the binding of anti-ILT7 antibodies can be assessed by incubating ILT7 expressing CHO cells with the anti-ILT7 antibody. The cells can be washed, and binding can be detected with an anti-human IgG Ab. Flow cytometric analyses can be performed using a FACScan flow cytometry instrument (Becton Dickinson, San Jose, CA). Mice which develop the highest titers can be used for fusions.

An ELISA assay as described above can be used to screen for antibodies and, thus, hybridomas that produce antibodies that show positive reactivity with the ILT7 immunogen. Hybridomas that produce antibodies that bind with high affinity to ILT7 can then be subcloned and further characterized. One clone from each hybridoma, which retains the reactivity of the parent cells (by ELISA), can then be chosen for making a cell bank, and for antibody purification.

To purify anti-ILT7 antibodies, selected hybridomas can be grown for monoclonal antibody purification. Supernatants can be filtered and concentrated before affinity chromatography. Eluted IgG can be checked by gel electrophoresis and high-performance liquid chromatography to ensure purity. The buffer solution can be exchanged, and the concentration can be determined. The monoclonal antibodies can be aliquoted and stored.

To determine if the selected anti-ILT7 monoclonal antibodies bind to unique epitopes, each antibody can be biotinylated using commercially available reagents (Pierce, Rockford, IL). Biotinylated MAb binding can be detected with a streptavidin labeled probe. Competition studies using unlabeled monoclonal antibodies and biotinylated monoclonal antibodies can be performed using ILT7 coated-ELISA plates as described above.

To determine the isotype of purified antibodies, isotype ELISAs can be performed using reagents specific for antibodies of a particular isotype. For example, to determine the isotype of a human monoclonal antibody, wells of microtiter plates can be coated with 1 pg/mL of anti-human immunoglobulin overnight at 4° C. After blocking with 1% BSA, the plates are reacted with test monoclonal antibodies or purified isotype control antibodies at ambient temperature for one to two hours. The wells can then be reacted with either human IgG1 or human IgM-specific alkaline phosphatase-conjugated probes. Plates are developed and analyzed as described above.

To test the binding of monoclonal antibodies to live cells expressing ILT7, flow cytometry can be used, as described in the Examples. Briefly, cell lines expressing membrane-bound ILT7 (grown under standard growth conditions) are mixed with various concentrations of monoclonal antibodies in PBS containing 0.1% BSA at 4° C. for 1 hour. After washing, the cells are reacted with Fluorescein-labeled anti-IgG antibody under the same conditions as the primary antibody staining. The samples can be analyzed by FACScan instrument using light and side scatter properties to gate on single cells and binding of the labeled antibodies is determined. An alternative assay using fluorescence microscopy can be used (in addition to or instead of) the flow cytometry assay. Cells can be stained exactly as described above and examined by fluorescence microscopy. This method allows visualization of individual cells, but can have diminished sensitivity depending on the density of the antigen.

Anti-ILT7 antibodies or antigen-binding fragments can be further tested for reactivity with the ILT7 antigen by Western blotting. Briefly, cell extracts from cells expressing ILT7 can be prepared and subjected to sodium dodecyl sulfate polyacrylamide gel electrophoresis. After electrophoresis, the separated antigens will be transferred to nitrocellulose membranes, blocked with 20% mouse serum, and probed with the monoclonal antibodies to be tested. IgG binding can be detected using anti-IgG alkaline phosphatase and developed with BCIP/NBT substrate tablets (Sigma Chem. Co., St. Louis, MO).

Methods for analyzing binding affinity, cross-reactivity, and binding kinetics of various anti-ILT7 antibodies include standard assays known in the art, for example, biolayer interferometry (BLI) using, for example, Gator system (Probe Life) or the Octet-96 system (Sartorius AG), or BIACORE™ surface plasmon resonance (SPR) analysis using a BIACORE™ 2000 SPR instrument (Biacore AB, Uppsala, Sweden).

### F. Pharmaceutical Compositions

Provided herein are also pharmaceutical compositions comprising the anti-ILT7 antibodies or antigen-binding fragments disclosed herein. In some embodiments, the pharmaceutical composition comprises a therapeutically effective amount of the anti-ILT7 antibodies or antigen-binding fragments disclosed herein and a pharmaceutically acceptable carrier. In some embodiments, the pharmaceutical compositions are useful in suppressing autoimmunity related to Type I IFN (e.g., IFNα) or pDCs. In some embodiments, the pharmaceutical compositions are useful in treating a disease or disorder associated with Type I IFN (e.g., IFNα) or pDCs.

In some embodiments, the pharmaceutical compositions provided herein comprise anti-ILT7 antibodies or antigen-binding fragments provided herein. The anti-ILT7 antibodies or antigen-binding fragments can be present at various concentrations. In some embodiments, the pharmaceutical compositions provided herein comprise soluble anti-ILT7 antibodies or antigen-binding fragments provided herein at 1-1000 mg/mL. Dosages can be readily adjusted by those skilled in the art; for example, a decrease in purity may require an increase in dosage.

Provided herein are also kits for preparation of pharmaceutical compositions having the anti-ILT7 antibodies or antigen-binding fragments disclosed herein. In some embodiments, the kit comprises the anti-ILT7 antibodies or antigen-binding fragments disclosed herein and a pharmaceutically acceptable carrier in one or more containers. In another embodiment, the kits can comprise anti-ILT7 antibodies or antigen-binding fragments disclosed herein for administration to a subject. In specific embodiments, the kits comprise instructions regarding the preparation and/or administration of the anti-ILT7 antibodies or antigen-binding fragments.

In some embodiments, provided herein is a pharmaceutical composition comprising anti-ILT7 antibodies or antigen-binding fragments or cells provided herein wherein the composition is suitable for local administration.

Pharmaceutically acceptable carriers that can be used in compositions provided herein include any and all solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal, or epidermal administration (e.g., by injection or infusion). Depending on the route of administration, the active ingredient (i.e., anti-ILT7 antibodies or antigen-binding fragments) can be coated in a material to protect the active ingredient from the action of acids and other natural conditions that can inactivate the active ingredient.

Provided herein are also pharmaceutical compositions or formulations that improve the stability of the anti-ILT7 antibodies or antigen-binding fragments to allow for their long-term storage. In some embodiments, the pharmaceutical composition or formulation disclosed herein comprises: (a) anti-ILT7 antibodies or antigen-binding fragments disclosed herein; (b) a buffering agent; (c) a stabilizing agent; (d) a salt; (e) a bulking agent; and/or (f) a surfactant. In some embodiments, the pharmaceutical composition or formulation is stable for at least 1 month, at least 2 months, at least 3 months, at least 6 months, at least 1 year, at least 2 years, at least 3 years, at least 5 years or more. In some embodiments, the pharmaceutical composition or formulation is stable when stored at 4° C., 25° C., or 40° C.

Buffering agents useful in the pharmaceutical compositions or formulations disclosed herein can be a weak acid or base used to maintain the acidity (pH) of a solution near a chosen value after the addition of another acid or base. Suitable buffering agents can maximize the stability of the pharmaceutical formulations by maintaining pH control of the formulation. Suitable buffering agents can also ensure physiological compatibility or optimize solubility. Rheology, viscosity, and other properties can also depend on the pH of the formulation. Common buffering agents include, but are not limited to, histidine, citrate, succinate, acetate, and phosphate. In some embodiments, a buffering agent comprises histidine (e.g., L-histidine) with isotonicity agents and potentially pH adjustment with an acid or a base known in the art. In certain embodiments, the buffering agent is L-histidine. In certain embodiments, the pH of the formulation is maintained between about 2 and about 10, or between about 4 and about 8.

Stabilizing agents are added to a pharmaceutical product to stabilize that product. Such agents can stabilize proteins in different ways. Common stabilizing agents include, but are not limited to, amino acids such as glycine, alanine, lysine, arginine, or threonine, carbohydrates such as glucose, sucrose, trehalose, raffinose, or maltose, polyols such as glycerol, mannitol, sorbitol, cyclodextrins or dextrans of any kind and molecular weight, or PEG. In some embodiments, the stabilizing agent is chosen to maximize the stability of the polypeptide in lyophilized preparations. In certain embodiments, the stabilizing agent is sucrose and/or arginine.

Bulking agents can be added to a pharmaceutical composition or formulation to add volume and mass to the product, thereby facilitating precise metering and handling thereof. Common bulking agents include, but are not limited to, lactose, sucrose, glucose, mannitol, sorbitol, calcium carbonate, or magnesium stearate.

Surfactants are amphipathic substances with lyophilic and lyophobic groups. A surfactant can be anionic, cationic, zwitterionic, or nonionic. Examples of nonionic surfactants include, but are not limited to, alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, or dodecyl dimethylamine oxide. In some embodiments, the surfactant is polysorbate 20 or polysorbate 80.

The pharmaceutical compositions disclosed herein can further comprise one or more of a buffer system, a preservative, a tonicity agent, a chelating agent, a stabilizer and/or a surfactant, as well as various combinations thereof. The use of preservatives, isotonic agents, chelating agents, stabilizers, and surfactants in pharmaceutical compositions is well-known to the skilled person. Reference may be made to Remington: The Science and Practice of Pharmacy, 19^{th} edition, 1995.

In some embodiments, the pharmaceutical composition is an aqueous formulation. Such a formulation is typically a solution or a suspension, but can also include colloids, dispersions, emulsions, and multi-phase materials. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In some embodiments, the pharmaceutical compositions disclosed herein are freeze-dried, to which the physician or the patient adds solvents and/or diluents prior to use.

Pharmaceutical compositions disclosed herein can also include a pharmaceutically acceptable antioxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that can be employed in the pharmaceutical compositions or formulations described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions described herein is contemplated. A pharmaceutical composition or formulation can comprise a preservative or can be devoid of a preservative. Supplementary active compounds can be incorporated into the compositions.

Pharmaceutical compositions or formulations typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, the compositions can include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated herein. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

The amount of active ingredient which can be combined with a carrier material in the pharmaceutical compositions or formulations disclosed herein can vary. In some embodiments, the amount of active ingredient which can be combined with a carrier material is the amount that produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

The pharmaceutical compositions disclosed herein can be prepared with carriers that protect the active ingredient against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and poly lactic acid. Many methods for the preparation of such formulations are patented or generally known to those skilled in the art. See. e.g., Sustained and Controlled Release Drug Delivery Systems, J. R. Robinson, ed., Marcel Dekker, Inc., New York, 1978.

In some embodiments, the anti-ILT7 antibodies or antigen-binding fragments described herein are formulated to ensure proper distribution in vivo. For example, the blood-brain barrier (BBB) excludes many highly hydrophilic compounds. To ensure that the activate ingredient described herein cross the BBB, they can be formulated, for example, in liposomes. For methods of manufacturing liposomes, see, e.g., U.S. Pat. Nos. 4,522,811; 5,374,548; and 5,399,331. The liposomes can comprise one or more moieties which are selectively transported into specific cells or organs, thus enhancing targeted drug delivery (see, e.g., V. V. Ranade (1989) J. Clin. Pharmacol. 29: 685). Exemplary targeting moieties include folate or biotin (see, e.g., U.S. Pat. No. 5,416,016 to Low et al) mannosides (Umezawa et al, (1988) Biochem. Biophys. Res. Commun. 153: 1038); antibodies (P. G. Bloeman et al. (1995) FEBS Lett. 357: 140; M. Owais et al. (1995) Antimicrob. Agents Chemother. 39: 180); surfactant protein A receptor (Briscoe et al. (1995) Am. J. Physiol. 1233: 134); and p120 (Schreier et al. (1994) J. Biol. Chem. 269: 9090); see also K. Keinanen; M. L. Laukkanen (1994) FEBS Lett. 346: 123; J. J. Killion; I. J. Fidler (1994) Immunomethods 4: 273.

### G. Methods and Uses

The antibodies or antigen-binding fragments, compositions, and methods described herein have numerous in vitro and in vivo utilities involving, for example, reducing Type I IFN release by ILT7-expression cells, such as pDCs. In some embodiments, the anti-ILT7 antibodies or antigen-binding fragments described herein are humanized antibodies or antigen-binding fragments. For example, anti-ILT7 antibodies or antigen-binding fragments described herein can be administered to cells in culture, in vitro or ex vivo, or to human subjects, e.g., in vivo, to selectively inhibit Type I IFN release or suppress activity of pDCs in a variety of diseases. Accordingly, provided herein are methods of reducing autoimmunity in a subject, the methods comprising administering to the subject an anti-ILT7 antibody or antigen binding portion described herein, such that the autoimmunity associated with Type 1 IFN or pDCs in the subject is reduced.

The present disclosure also provides methods of uses of the anti-ILT7 antibodies or antigen-binding fragments, polynucleotides encoding such anti-ILT7 antibodies or antigen-binding fragments, vectors comprising such polynucleotides, or pharmaceutical compositions having such antibodies or antigen-binding fragments disclosed herein in reducing cytokine (e.g., Type I IFN) release by ILT7-expressing cells, such as pDCs, or treating a disease or disorder associated with Type I IFN-associated or pDC-associated diseases, such as autoimmune diseases.

In some embodiments, provided herein are methods of reducing Type I IFN (e.g., IFNα) in a subject in need thereof, the methods comprising administering to the subject a therapeutically effective amount of the anti-ILT7 antibodies or antigen-binding fragments disclosed herein. In some embodiments, provided herein are uses of the anti-ILT7 antibodies or antigen-binding fragments disclosed herein for reducing Type I IFN (e.g., IFNα). In some embodiments, provided herein are uses of the anti-ILT7 antibodies or antigen-binding fragments provided herein for the preparation of a medicament for reducing Type I IFN (e.g., IFNα). In some embodiments, provided herein are methods of reducing Type I IFN (e.g., IFNα) in a subject in need thereof, the methods comprising administering to the subject a therapeutically effective amount of the pharmaceutical compositions disclosed herein. In some embodiments, provided herein are uses of the pharmaceutical compositions disclosed herein for reducing Type I IFN (e.g., IFNα). In some embodiments, provided herein are uses of the pharmaceutical compositions provided herein for the preparation of a medicament for reducing Type I IFN (e.g., IFNα).

In some embodiments, provided herein are methods of suppressing or depleting pDCs in a subject in need thereof, the methods comprising administering to the subject a therapeutically effective amount of the anti-ILT7 antibodies or antigen-binding fragments disclosed herein. In some embodiments, provided herein are uses of the anti-ILT7 antibodies or antigen-binding fragments disclosed herein for suppressing or depleting pDCs. In some embodiments, provided herein are uses of the anti-ILT7 antibodies or antigen-binding fragments provided herein for the preparation of a medicament for suppressing or depleting pDCs. In some embodiments, provided herein are methods of suppressing or depleting pDCs in a subject in need thereof, the methods comprising administering to the subject a therapeutically effective amount of the pharmaceutical compositions disclosed herein. In some embodiments, provided herein are uses of the pharmaceutical compositions disclosed herein for suppressing or depleting pDCs. In some embodiments, provided herein are uses of the pharmaceutical compositions provided herein for the preparation of a medicament for suppressing or depleting pDCs. In some embodiments, the anti-ILT7 antibodies or antigen-binding fragments disclosed herein deplete pDCs in a subject in need thereof.

In some embodiments, provided herein are methods of reducing autoimmunity associated with Type I IFN (e.g., IFNα) or pDCs in a subject in need thereof, the methods comprising administering to the subject a therapeutically effective amount of the anti-ILT7 antibodies or antigen-binding fragments disclosed herein. In some embodiments, provided herein are uses of the anti-ILT7 antibodies or antigen-binding fragments disclosed herein for reducing autoimmunity associated with Type I IFN (e.g., IFNα) or pDCs. In some embodiments, provided herein are uses of the anti-ILT7 antibodies or antigen-binding fragments provided herein for the preparation of a medicament for reducing autoimmunity associated with Type I IFN (e.g., IFNα) or pDCs. In some embodiments, provided herein are methods of reducing autoimmunity associated with Type I IFN (e.g., IFNα) or pDCs in a subject in need thereof, the methods comprising administering to the subject a therapeutically effective amount of the pharmaceutical compositions disclosed herein. In some embodiments, provided herein are uses of the pharmaceutical compositions disclosed herein for reducing autoimmunity associated with Type I IFN (e.g., IFNα) or pDCs. In some embodiments, provided herein are uses of the pharmaceutical compositions provided herein for the preparation of a medicament for reducing autoimmunity associated with Type I IFN (e.g., IFNα) or pDCs.

In some embodiments, provided herein are methods of treating an autoimmune disease associated with Type I IFN (e.g., IFNα) or pDCs in a subject in need thereof, the methods comprising administering to the subject a therapeutically effective amount of the anti-ILT7 antibodies or antigen-binding fragments disclosed herein. In some embodiments, provided herein are uses of the anti-ILT7 antibodies or antigen-binding fragments disclosed herein for treating an autoimmune disease associated with Type I IFN (e.g., IFNα) or pDCs. In some embodiments, provided herein are uses of the anti-ILT7 antibodies or antigen-binding fragments provided herein for the preparation of a medicament for treating an autoimmune disease associated with Type I IFN (e.g., IFNα) or pDCs. In some embodiments, provided herein are methods of treating an autoimmune disease associated with Type I IFN (e.g., IFNα) or pDCs in a subject in need thereof, the methods comprising administering to the subject a therapeutically effective amount of the pharmaceutical compositions disclosed herein. In some embodiments, provided herein are uses of the pharmaceutical compositions disclosed herein for treating an autoimmune disease associated with Type I IFN (e.g., IFNα) or pDCs. In some embodiments, provided herein are uses of the pharmaceutical compositions provided herein for the preparation of a medicament for treating an autoimmune disease associated with Type I IFN (e.g., IFNα) or pDCs.

As known in the art, dysregulated or excessive activation of pDCs or release of Type I IFN (e.g., IFNα) leads to unwanted activation of the immune system, which has been implicated in the pathogenesis of a wide variety of diseases, such as autoimmune diseases. In some embodiments, the disease or disorder associated with pDCs or IFNα that can be treated with the anti-ILT7 antibodies or antigen-binding fragments, or pharmaceutical compositions provided herein includes lupus; lupus erythematosus such as systemic lupus erythematosus (SLE), cutaneous lupus erythematosus (CLE) and discoid lupus erythematosus (DLE); and/or lupus nephritis (LN). In some embodiments, the disease or disorder that can be treated with the anti-ILT7 antibodies or antigen-binding fragments, or pharmaceutical compositions provided herein is lupus. In some embodiments, the disease or disorder that can be treated with the anti-ILT7 antibodies or antigen-binding fragments, or pharmaceutical compositions provided herein is SLE. In some embodiments, the disease or disorder that can be treated with the anti-ILT7 antibodies or antigen-binding fragments, or pharmaceutical compositions provided herein is CLE. In some embodiments, the disease or disorder that can be treated with the anti-ILT7 antibodies or antigen-binding fragments, or pharmaceutical compositions provided herein is DLE. In some embodiments, the disease or disorder that can be treated with the anti-ILT7 antibodies or antigen-binding fragments, or pharmaceutical compositions provided herein is LN.

In some embodiments, the methods provided herein promote a beneficial therapeutic response with respect to an autoimmune response. In some embodiments, methods provided herein result in an improvement of symptoms associated with a disease, such as a decrease in IFNα levels, a decrease in the number or activity of pDCs, or a decrease in one or more other symptoms associated with the disease. Thus, for example, an improvement in the disease can be characterized as a complete response. In some embodiments, a clinical response can be assessed using screening techniques such as magnetic resonance imaging (MRI) scanning, x-radiographic imaging, computed tomographic (CT) scanning, flow cytometry or fluorescence-activated cell sorter (FACS) analysis, histology, gross pathology, and blood chemistry, including but not limited to techniques measuring changes detectable by ELISA, RIA, chromatography, and the like.

Actual dosage levels of the active ingredients (i.e., the anti-ILT7 antibodies or antigen-binding fragments) in the pharmaceutical compositions described herein can be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions described herein, the route of administration, the time of administration, the rate of excretion, the duration of the treatment, other drugs, compounds and/or materials used in combination with the particular compositions employed, the age, sex, weight, condition, general health, and prior medical history of the patient being treated, and like factors well known in the medical arts.

The anti-ILT7 antibodies or antigen-binding fragments can be administered as a sustained release formulation, in which case less frequent administration is required. Dosage and frequency vary depending on the half-life of the anti-ILT7 antibodies or antigen-binding fragments in the patient. In therapeutic applications, a relatively high dosage at relatively short intervals is sometimes required until progression of the disease is reduced or terminated, and until the patient shows partial or complete amelioration of symptoms of disease.

The anti-ILT7 antibodies or antigen-binding fragments or pharmaceutical compositions provided herein can be administered to a subject by any methods known in the art, including, but not limited to, pleural administration, intravenous administration, subcutaneous administration, intranodal administration, intramuscular administration, intradermal administration, intrathecal administration, intrapleural administration, intraperitoneal administration, intracranial administration, spinal or other parenteral routes of administration, for example by injection or infusion, or direct administration to the thymus. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection, and infusion. In some embodiments, subcutaneous administration is adopted. In some embodiments, intravenous administration is adopted. In some embodiments, oral administration is adopted. In one embodiment, the antibodies or antigen-binding fragments provided herein are delivered locally. In another embodiment, the antibodies or antigen-binding fragments provided herein are administered systemically.

In the methods disclosed herein, a therapeutically effective amount of the anti-ILT7 antibodies or antigen-binding fragments or pharmaceutical compositions disclosed herein is administered to a subject that can benefit from reduction in the IFNα level. The subject can have unwanted, unregulated, or excessive activation of pDCs. The subject can be a mammal. In some embodiments, the subject is a human.

Anti-ILT7 antibodies or antigen-binding fragments or pharmaceutical compositions provided herein can be administered with medical devices known in the art. For example, in some embodiments, a needleless hypodermic injection device can be used, such as the devices disclosed in U.S. Pat. Nos. 5,399,163; 5,383,851; 5,312,335; 5,064,413; 4,941,880; 4,790,824; or 4,596,556. Examples of well-known implants and modules for use described herein include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In some embodiments, the anti-ILT7 antibodies or antigen-binding fragments or pharmaceutical compositions provided herein are administered with an additional therapy. The additional therapy can be administered prior to, concurrently with, or subsequent to administration of the anti-ILT7 antibodies or antigen-binding fragments, cells, or pharmaceutical compositions described herein. Combined administration can include co-administration, either in a single pharmaceutical formulation or using separate formulations, or consecutive administration in either order but generally within a time period such that all active agents can exert their biological activities simultaneously. A person skilled in the art can readily determine appropriate regimens for administering a pharmaceutical composition described herein and an additional therapy in combination, including the timing and dosing of an additional agent to be used in a combination therapy, based on the needs of the subject being treated.

The antibodies or antigen-binding fragments provided herein can also be used in detection of ILT7. Also encompassed are methods for detecting the presence of human ILT7 antigen in a sample, or measuring the amount of human ILT7 antigen, comprising contacting the sample, and a control sample, with a monoclonal antibody, e.g., a humanized monoclonal antibody, or an antigen binding portion thereof, which specifically binds to human ILT7, under conditions that allow for formation of a complex between the antibody or antigen-binding fragment and human ILT7. The formation of a complex is then detected, wherein a difference complex formation between the sample compared to the control sample is indicative the presence of human ILT7 antigen in the sample. Moreover, the anti-ILT7 antibodies or antigen-binding fragments described herein can be used to purify human ILT7 via immunoaffinity purification. In some embodiments, the anti-ILT7 antibodies or antigen-binding fragments described herein are used for detecting ILT7-expressing cells, such as pDCs. In some embodiments, the anti-ILT7 antibodies or antigen-binding fragments described herein are used for quantifying ILT7 antigen, or ILT7-expressing cells.

### H. Exemplified Embodiments

Embodiment 1: An antibody or antigen-binding fragment thereof that specifically binds human ILT7, the antibodies or antigen-binding fragments comprising: (1) as defined by Kabat, (a) a light chain variable region (VL) comprising VL CDR1, VL CDR2, and VL CDR3 having the amino acid sequences of SEQ ID NOs: 11, 12, and 13, respectively; or a variant thereof having up to about 5 amino acid substitutions, additions, and/or deletions in the VL CDRs; and/or (b) a heavy chain variable region (VH) comprising VH CDR1, VH CDR2, and VH CDR3 having the amino acid sequences of SEQ ID NOs: 14, 15, and 16, respectively; or a variant thereof having up to about 5 amino acid substitutions, additions, and/or deletions in the VH CDRs; or (2) as defined by Chothia, (a) a VL comprising VL CDR1, VL CDR2, and VL CDR3 having the amino acid sequences of SEQ ID NOs: 11, 12, and 13, respectively; or a variant thereof having up to about 5 amino acid substitutions, additions, and/or deletions in the VL CDRs; and/or (b) a VH comprising VH CDR1, VH CDR2, and VH CDR3 having the amino acid sequences of SEQ ID NOs: 17, 18, and 16, respectively; or a variant thereof having up to about 5 amino acid substitutions, additions, and/or deletions in the VH CDRs.

Embodiment 2: The antibody or antigen-binding fragment of Embodiment 1, comprising VL CDR1, VL CDR2, VL CDR3, VH CDR1, VH CDR2 and VH CDR3 having the amino acid sequences of SEQ ID NOs: 11, 12, 13, 14, 15, and 16, respectively, as defined by Kabat.

Embodiment 3: The antibody or antigen-binding fragment of Embodiment 1, comprising VL CDR1, VL CDR2, VL CDR3, VH CDR1, VH CDR2 and VH CDR3 having the amino acid sequences of SEQ ID NOs: 11, 12, 13, 17, 18, and 16, respectively, as defined by Chothia.

Embodiment 4: An antibody or antigen-binding fragment thereof that specifically binds human ILT7, the antigen or antigen-binding fragment comprising: (a) a VL having at least 85%, at least 90%, at least 95%, at least 98%, or 100% sequence identity to the amino acid sequence of SEQ ID NO: 9; and/or (b) a VH having at least 85%, at least 90%, at least 95%, at least 98%, or 100% sequence identity to the amino acid sequence of SEQ ID NO: 10.

Embodiment 5: The antibody or antigen-binding fragment of Embodiment 4, comprising a VL and a VH having the amino acid sequences of SEQ ID NOs: 9 and 10, respectively.

Embodiment 6: An antibody or antigen-binding fragment thereof that specifically binds human ILT7, the antigen or antigen-binding fragment comprising (a) a VL comprising VL CDR1, VL CDR2, and VL CDR3 from a VL having the amino acid sequence of SEQ ID NO: 9; and/or (b) a VH comprising VH CDR1, VH CDR2, and VH CDR3 from a VH having the amino acid sequence of SEQ ID NO: 10.

Embodiment 7: The antibody or antigen-binding fragment of any one of Embodiments 1 to 6, wherein the antibody or antigen-binding fragment is a chimeric antibody or antigen-binding fragment, a humanized antibody or antigen-binding fragment, or a human antibody or antigen-binding fragment.

Embodiment 8: The antibody or antigen-binding fragment of Embodiment 7, wherein the antibody or antigen-binding fragment is a humanized antibody or antigen-binding fragment.

Embodiment 9: The antibody or antigen-binding fragment of Embodiment 8, comprising: (a) a VL having at least 85%, at least 90%, at least 95%, at least 98%, or 100% sequence identity to the amino acid sequence of any one of SEQ ID NOs: 19-22; and/or (b) a VH having at least 85%, at least 90%, at least 95%, at least 98%, or 100% sequence identity to the amino acid sequence of any one of SEQ ID NOs: 23-28.

Embodiment 10: The antibody or antigen-binding fragment of Embodiment 9, comprising: (a) a VL having at least 85%, at least 90%, at least 95%, at least 98%, or 100% sequence identity to the amino acid sequence of SEQ ID NO: 19; and (b) a VH having at least 85%, at least 90%, at least 95%, at least 98%, or 100% sequence identity to the amino acid sequence of SEQ ID NO: 26.

Embodiment 11: The antibody or antigen-binding fragment of Embodiment 9, comprising: (a) a VL having at least 85%, at least 90%, at least 95%, at least 98%, or 100% sequence identity to the amino acid sequence of SEQ ID NO: 19, the VL comprising, as defined by Kabat or Chothia, VL CDR1, VL CDR2, and VL CDR3 having the amino acid sequences of SEQ ID NOs: 11, 12, and 13, respectively; and (b) a VH having at least 85%, at least 90%, at least 95%, at least 98%, or 100% sequence identity to the amino acid sequence of SEQ ID NO: 26, the VH comprising (1) as defined by Kabat, VH CDR1, VH CDR2, and VH CDR3 having the amino acid sequences of SEQ ID NOs: 14, 15, and 16, respectively, or (2) as defined by Chothia, VH CDR1, VH CDR2, and VH CDR3 having the amino acid sequences of SEQ ID NOs: 17, 18, and 16, respectively.

Embodiment 12: The antibody or antigen-binding fragment of Embodiment 9, comprising a VL and a VH having the amino acid sequences of (1) SEQ ID NOs: 19 and 23, respectively; (2) SEQ ID NOs: 19 and 24, respectively; (3) SEQ ID NOs: 19 and 25, respectively; (4) SEQ ID NOs: 19 and 26, respectively; (5) SEQ ID NOs: 19 and 27, respectively; (6) SEQ ID NOs: 19 and 28, respectively; (7) SEQ ID NOs: 20 and 23, respectively; (8) SEQ ID NOs: 20 and 24, respectively; (9) SEQ ID NOs: 20 and 25, respectively; (10) SEQ ID NOs: 20 and 26, respectively; (11) SEQ ID NOs: 20 and 27, respectively; (12) SEQ ID NOs: 20 and 28, respectively; (13) SEQ ID NOs: 21 and 23, respectively; (14) SEQ ID NOs: 21 and 24, respectively; (15) SEQ ID NOs: 21 and 25, respectively; (16) SEQ ID NOs: 21 and 26, respectively; (17) SEQ ID NOs: 21 and 27, respectively; (18) SEQ ID NOs: 21 and 28, respectively; (19) SEQ ID NOs: 22 and 23, respectively; (20) SEQ ID NOs: 22 and 24, respectively; (21) SEQ ID NOs: 22 and 25, respectively; (22) SEQ ID NOs: 22 and 26, respectively; (23) SEQ ID NOs: 22 and 27, respectively; or (24) SEQ ID NOs: 22 and 28, respectively.

Embodiment 13: The antibody or antigen-binding fragment of Embodiment 12, comprising a VL having the amino acid sequence of SEQ ID NO: 19 and a VH having the amino acid sequence of SEQ ID NOs: 26.

Embodiment 14: The antibody or antigen-binding fragment of any one of Embodiments 1 to 13, wherein the antibody or antigen-binding fragment is a Fab, a Fab′, a F(ab′)2, a Fv, a scFv, a (scFv)₂, a single domain antibody (sdAb), or a heavy chain antibody (HCAb).

Embodiment 15: The antibody or antigen-binding fragment of any one of Embodiments 1 to 13, wherein the antibody or antigen-binding fragment is an IgG1 antibody, an IgG2 antibody, an IgG3 antibody, or an IgG4 antibody.

Embodiment 16: The antibody or antigen-binding fragment of Embodiment 15, wherein the antibody is an IgG1 antibody.

Embodiment 17: The antibody or antigen-binding fragment of Embodiment 16, comprising a light chain constant region (CL) having at least 85% sequence identity to kappa CL (CK; SEQ ID NO: 29).

Embodiment 18: The antibody or antigen-binding fragment of Embodiment 16, comprising a light chain constant region (CL) having at least 85% sequence identity to lambda CL (Ck; SEQ ID NO: 30).

Embodiment 19: The antibody or antigen-binding fragment of Embodiment 16, comprising a heavy chain constant region (CH) having at least 85% sequence identity to the amino acid sequence of any one of SEQ ID NOs: 31 and 40-44.

Embodiment 20: The antibody or antigen-binding fragment of any one of Embodiments 16 to 19, wherein the heavy chain constant region (CH) of the IgG1 antibody comprises a wildtype IgG1 CH, or comprises at least one amino acid mutation that enhances ADCC (antibody-dependent cellular cytotoxicity) or ADCP (antibody-dependent cellular phagocytosis) of the antibody.

Embodiment 21: The antibody or antigen-binding fragment of Embodiment 20, wherein the CH region of the IgG1 antibody has an amino acid substitution at L234, L235, G236, S239, F243, H268, D270, R292, S298, Y300, V305, K326, A330, 1332, E333, K334, P396, or any combination thereof, numbered according to the EU Index.

Embodiment 22: The antibody or antigen-binding fragment of Embodiment 20, wherein the CH region of the IgG1 antibody has an amino acid substitution that is L234Y, L235Q, L235V, G236A, G236W, S239D, S239M, F243L, H268D, D270E, R292P, S298A, Y300L, V305I, K326D, A330M, A330L, I332E, E333A, K334A, K334E, or P396L, or any combination thereof, numbered according to the EU Index.

Embodiment 23: The antibody or antigen-binding fragment of Embodiment 20, wherein the CH region of the IgG1 antibody is modified by amino acid substitutions that are (i) S298A, E333A, and K334A; (ii) S239D and I332E; (iii) S239D, A330L, and I332E; (iv) G236A; (v) G236A, S239D, and I332E; (vi) G236A, A330L, and I332E; (vii) G236A, S239D, A330L, and I332E; (viii) F243L, R292P, Y300L, V305I, and P396L; (ix) L235V, F243L, R292P, Y300L, and P396L; (x) L234Y, L235Q, G236W, S239M, H268D, D270E, and S298A; or (xi) D270E, K326D, A330M, and K334E, numbered according to the EU Index.

Embodiment 24: The antibody or antigen-binding fragment of 23, wherein the CH region has the amino acid sequence selected from the group consisting of SEQ ID NOs: 45-64.

Embodiment 25: The antibody or antigen-binding fragment of Embodiment 1, comprising: a VL having the amino acid sequence of SEQ ID NO: 19; a VH having the amino acid sequence of SEQ ID NO: 26; and a CH having the amino acid sequence of SEQ ID NO: 55.

Embodiment 26: The antibody or antigen-binding fragment of Embodiment 1, comprising: a VL having the amino acid sequence of SEQ ID NO: 19; a VH having the amino acid sequence of SEQ ID NO: 26; a CL having the amino acid sequence of SEQ ID NO: 30; and a CH having the amino acid sequence of SEQ ID NO: 55.

Embodiment 27: The antibody or antigen-binding fragment of any one of Embodiments 16 to 26, wherein the Fe region of the IgG1 antibody is afucosylated.

Embodiment 28: An antibody or antigen-binding fragment thereof that competes with the antibody or antigen-binding fragment of any one of Embodiments 1 to 27 for binding to human ILT7.

Embodiment 29: The antibody or antigen-binding fragment of any one of Embodiments 1 to 28, wherein the antibody or antigen-binding fragment is a bispecific antibody or a multispecific antibody.

Embodiment 30: The antibody or antigen-binding fragment of any one of Embodiments 1 to 29, wherein the antibody or antigen-binding fragment is a monoclonal antibody or antigen-binding fragment.

Embodiment 31: The antibody or antigen-binding fragment of any one of Embodiments 1 to 30, wherein the antibody or antigen binding fragment: (1) binds to human ILT7 with a K_{D} of 500 nM or less, as measured by SPR; (2) does not specifically bind to LILR family member LILRA1, LILRA2/ILT1, LILRA3/ILT6, LILRA5/ILT11, LILRA6/ILT8, LILRB1/ILT2, LILRB2/ILT4, LILRB3/ILT5, LILRB4/ILT3, or LILB5; (3) inhibits interferon alpha (IFNα) release by peripheral blood mononuclear cells (PBMCs); (4) selectively binds to plasmacytoid dendritic cells (pDCs) in human PBMC; (5) exhibits natural killer cell (NK)-dependent ADCC activity against ILT7-expressing cells; (6) exhibits neutrophil-dependent ADCC activity against ILT7-expressing cells; or (7) exhibits macrophage-dependent ADCP activity against ILT7-expressing cells; or any combination of (1)-(7).

Embodiment 32: An antibody or antigen-binding fragment thereof that specifically binds the protease domain of human ILT7, wherein the antibody or antigen binding fragment: (1) binds to human ILT7 with a K_{D} of 500 nM or less, as measured by SPR; (2) does not specifically bind to LILR family member LILRA1, LILRA2/ILT1, LILRA3/ILT6, LILRA5/ILT11, LILRA6/ILT8, LILRB1/ILT2, LILRB2/ILT4, LILRB3/ILT5, LILRB4/ILT3, or LILB5; (3) inhibits IFNα release by PBMCs; (4) selectively binds to pDCs in human PBMCs; (5) exhibits NK-dependent ADCC activity against ILT7-expressing cells; (6) exhibits neutrophil-dependent ADCC activity against ILT7-expressing cells; or (7) exhibits macrophage-dependent ADCP activity against ILT7-expressing cells; or any combination of (1)-(7).

Embodiment 33: The antibody or antigen-binding fragment of Embodiment 31 or 32, wherein the antibody or antigen-binding fragment (1) inhibits IFNα release by CpG-stimulated PBMCs in vitro with an EC50 of 1 nM or less; (2) exhibits NK-dependent ADCC activity against ILT7-expressing cells with an EC50 of 0.01 nM or less; (3) exhibits neutrophil-dependent ADCC activity against ILT7-expressing cells with an EC50 of 100 nM or less; (4) exhibits macrophage-dependent ADCP activity against ILT7-expressing cells with an EC50 of 10 nM or less; or (5) exhibits macrophage-dependent ADCP activity against ILT7-expressing cells with a maximum phagocytic index of 20% or higher; or any combination of (1)-(5).

Embodiment 34: The antibody or antigen-binding fragment of Embodiment 33, wherein the antibody or antigen-binding fragment (1) inhibits IFNα release by PBMCs with an EC50 ranging from 0.01 nM to 0.1 nM; (2) exhibits NK-dependent ADCC activity against ILT7-expressing cells with an EC50 ranging from 0.001 nM to 0.01 nM; (3) exhibits neutrophil-dependent ADCC activity against ILT7-expressing cells with an EC50 ranging from 1 nM to 50 nM; (4) exhibits macrophage-dependent ADCP activity against ILT7-expressing cells with an EC50 ranging from 0.5 nM to 5 nM; or (5) exhibits macrophage-dependent ADCP activity against ILT7-expressing cells with a maximum phagocytic index ranging from 20% to 80%; or any combination of (1)-(5).

Embodiment 35: The antibody or antigen-binding fragment of any one of Embodiments 31 to 34, wherein the antigen or antigen-binding fragment exhibits neutrophil-dependent ADCC activity.

Embodiment 36: A polynucleotide encoding a polypeptide of the antibody or antigen-binding fragment of any one of Embodiments 1 to 35.

Embodiment 37: A vector comprising the polynucleotide of Embodiment 36.

Embodiment 38: A host cell comprising the polynucleotide of Embodiment 36, or the vector of Embodiment 37.

Embodiment 39: The host cell of Embodiment 38, wherein the host cell (1) overexpresses N-acetylglucosaminyltransferase III (GnTIII), (2) lacks a-1,6-fucosyltransferase (FUT8), or (3) has a low fucose content, or any combination of (1)-(3).

Embodiment 40: A method of making an antibody or antigen-binding fragment thereof that specifically binds human ILT7, the method comprising culturing the host cell of Embodiment 38 or 39 in a culture under conditions that allow expression of the antibody or antibody fragment.

Embodiment 41: The method of Embodiment 40, further comprising isolating the antibody from the culture.

Embodiment 42: A pharmaceutical composition comprising a therapeutically effective amount of the antibody or antigen-binding fragment of any one of Embodiments 1 to 35, and a pharmaceutically acceptable carrier.

Embodiment 43: A method of reducing Type I interferon (IFN) in a subject in need thereof, the method comprising administering to the subject a therapeutically effective amount of the antibody or antigen-binding fragment of any one of Embodiments 1 to 35.

Embodiment 44: The method of Embodiment 43, wherein the Type I interferon is IFNα.

Embodiment 45: A method of suppressing or depleting pDCs in a subject in need thereof, the method comprising administering to the subject a therapeutically effective amount of the antibody or antigen-binding fragment of any one of Embodiments 1 to 35.

Embodiment 46: A method of reducing autoimmunity in a subject in need thereof, the method comprising administering to the subject an effective amount of the antibody or antigen-binding fragment of any one of Embodiments 1 to 35.

Embodiment 47: The method of any one of Embodiments 43 to 46, wherein the subject has an autoimmune disease.

Embodiment 48: A method of treating an autoimmune disease associated with Type I IFN or pDCs in a subject in need thereof, the method comprising administering to the subject a therapeutically effective amount of the antibody or antigen-binding fragment of any one of Embodiments 1 to 35.

Embodiment 49: The method of Embodiment 47 or 48, wherein the autoimmune disease is systemic lupus erythematosus (SLE).

Embodiment 50: The method of any one of Embodiments 43 to 49, further comprising administering an additional therapy to the subject.

Embodiment 51: The method of any one of Embodiments 43 to 50, wherein the subject is a human.

Embodiment 52: Use of the antibody or antigen-binding fragment of any one of Embodiments 1 to 35 in reducing Type I IFN.

Embodiment 53: Use of the antibody or antigen-binding fragment of any one of Embodiments 1 to 35 for preparation of a medicament for reducing Type I IFN.

Embodiment 54: The use of Embodiment 52 or 53, wherein the Type I IFN is IFNα.

Embodiment 55: Use of the antibody or antigen-binding fragment of any one of Embodiments 1 to 35 in suppressing or depleting pDCs.

Embodiment 56: Use of the antibody or antigen-binding fragment of any one of Embodiments 1 to 35 for preparation of a medicament for suppressing or depleting pDCs.

Embodiment 57: Use of the antibody or antigen-binding fragment of any one of Embodiments 1 to 35 in reducing autoimmunity.

Embodiment 58: Use of the antibody or antigen-binding fragment of any one of Embodiments 1 to 35 for preparation of a medicament for reducing autoimmunity.

Embodiment 59: Use of the antibody or antigen-binding fragment of any one of Embodiments 1 to 35 in treating an autoimmune disease associated with Type I IFN or pDCs.

Embodiment 60: Use of the antibody or antigen-binding fragment of any one of Embodiments 1 to 35 for preparation of a medicament for treating an autoimmune disease associated with Type I IFN or pDCs.

Embodiment 61: The use of Embodiment 59 or 60, wherein the autoimmune disease is SLE.

The practice of the invention employs, unless otherwise indicated, conventional techniques in molecular biology, cell biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) MOLECULAR CLONING: A LABORATORY MANUAL, Cold Spring Harbor Laboratory Press; Sambrook et al. (1989), MOLECULAR CLONING: A LABORATORY MANUAL, Second Edition, Cold Spring Harbor Laboratory Press; Sambrook et al. (2001) MOLECULAR CLONING: A LABORATORY MANUAL, Cold Spring Harbor Laboratory Press, Cold Spring Harbor, NY; Ausubel et al., CURRENT PROTOCOLS IN MOLECULAR BIOLOGY, John Wiley & Sons (1987 and annual updates); CURRENT PROTOCOLS IN IMMUNOLOGY, John Wiley & Sons (1987 and annual updates) Gait (ed.) (1984) OLIGONUCLEOTIDE SYNTHESIS: A PRACTICAL APPROACH, IRL Press; Eckstein (ed.) (1991) OLIGONUCLEOTIDES AND ANALOGUES: A PRACTICAL APPROACH, IRL Press; Birren et al. (eds.) (1999) GENOME ANALYSIS: A LABORATORY MANUAL, Cold Spring Harbor Laboratory Press; Borrebaeck (ed.) (1995) ANTIBODY ENGINEERING, Second Edition, Oxford University Press; Lo (ed.) (2006) ANTIBODY ENGINEERING: METHODS AND PROTOCOLS (METHODS IN MOLECULAR BIOLOGY); Vol. 248, Humana Press, Inc; each of which is incorporated herein by reference in its entirety.

### EXAMPLES

The examples provided below are for purposes of illustration only, and are not intended to be limiting unless otherwise specified. Thus, the invention should in no way be construed as being limited to the following examples, but rather, should be construed to encompass any and all variations which become evident as a result of the teaching provided herein.

Briefly, results from the studies described below demonstrate that cmAb12 and its humanized versions bind to human ILT7 with high affinity and are more potent than the benchmark antibody daxdilimab (“Tab1”; heavy chain: SEQ ID NO: 38; and light chain: SEQ ID NO: 39) in blocking IFNα release and suppressing pDC.

### Example 1: Immunization and Antibody Screening

Methods: For production of Anti-ILT7 antibodies, Balb/c and SJL mice were immunized by three different strategies. First, human ILT7-his protein was generated by fusing a his6 tag to the N-terminus of the extracellular domain of human ILT7 (amino acids 24-446 of SEQ ID NO: 1); second, a vector containing the sequence encoding full-length human ILT7 (SEQ ID NO: 5) linked to the sequence encoding hFcεRiγ (SEQ ID NO: 7) was generated and used for gene immunization and lentivirus packaging; and third, HEK-293F cells were infected with lentivirus to generate a stable clone of full-length human ILT7 to obtain 293F-human ILT7 cells. All the materials mentioned above were used as immunogens. In particular, human ILT7-his protein was administered by hock injection. Serum titers were examined for the first round of screening and antibodies from mice with a strong immune response against ILT7 were selected for hybridoma generation. The selected monoclonal antibodies were then re-screened for their binding affinities to ILT7 using FACS and ELISA, and for their activities in reducing IFNα release using IFNα release assay.

### Example 2: Generation and Characterization of Chimeric Antibodies

Methods: From the hybridoma screening, 25 antibodies were expressed and purified. The vectors expressing the heavy chains and light chains of the chimeric antibodies were constructed. The heavy chain expression vectors contained the coding sequences for the heavy chain variable domains of the chimeric antibodies linked to the coding sequence for the heavy chain constant region of human IgG1. Likewise, the light chain expression vectors contained the coding sequences for the light chain variable domains of the chimeric antibodies and the coding sequence for the constant region of κ light chain.

### Example 3: Binding of cmAb12 to ILT7 Measured by ELISA

Methods: The binding of the chimeric anti-ILT7 antibodies identified from the screening, including cmAb12, to human ILT7-his protein was measured by ELISA, along with an isotype antibody hIgG1 as negative control and a reference anti-ILT7 antibody Tab1. 96-well ELISA plates were coated with human ILT7-his in PBS (2 μg/ml) overnight at 4° C. Chimeric antibodies were added to the wells and co-incubated with the coating protein at 37° C. for 1 hour, followed by the addition of secondary HRP labelled antibody. After incubation at 37° C. for 1 more hour, TMB substrate was added to wells and incubated at RT for 15 min. 1 N HCl was added to terminate the reactions. The absorbance at 450 nm was measured with a microplate reader.

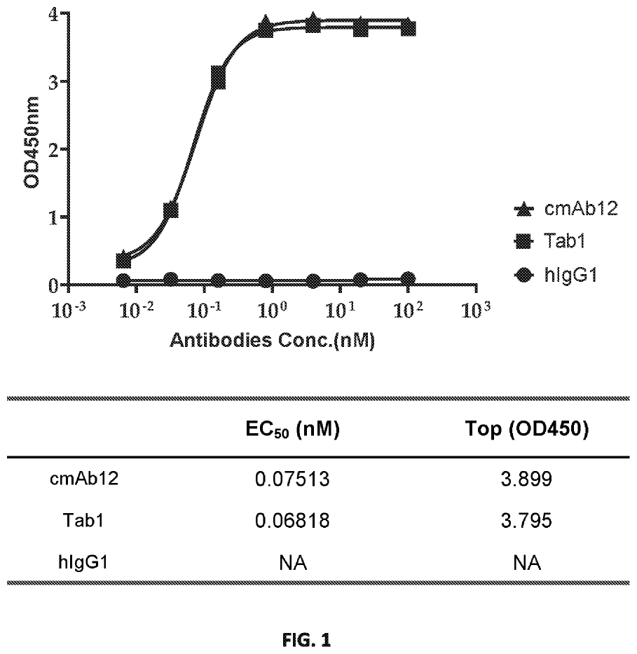

Results and conclusions: As shown in FIG. 1, cmAb12 bound to human ILT7 protein with high affinity (EC₅₀ of 0.07513 nM).

### Example 4: Binding of cmAb12 to ILT7-Expressing Cells Measured by Flow Cytometry

Methods: The binding affinities of chimeric anti-ILT7 antibodies identified from the screening, including cmAb12, to human ILT7 or cynomolgus ILT7 expressed on cell membrane were measured by flow cytometry. Vectors containing coding sequences for full-length cynomolgus ILT7 (SEQ ID NO: 6) and hFcεRiγ (SEQ ID NO: 7) were generated. CHOK1-cynomolgus ILT7 stable cell line was similarly constructed as the 293F-human ILT7 cell. Both cell lines were seeded, resuspended, and incubated with the chimeric antibodies at 4° C. for 1 hour. Cells were then washed with cold FACS buffer (PBS+2% FBS), resuspended, and incubated with secondary antibody at 4° C. for 1 hour. hIgG1 and Tab1 were used as controls.

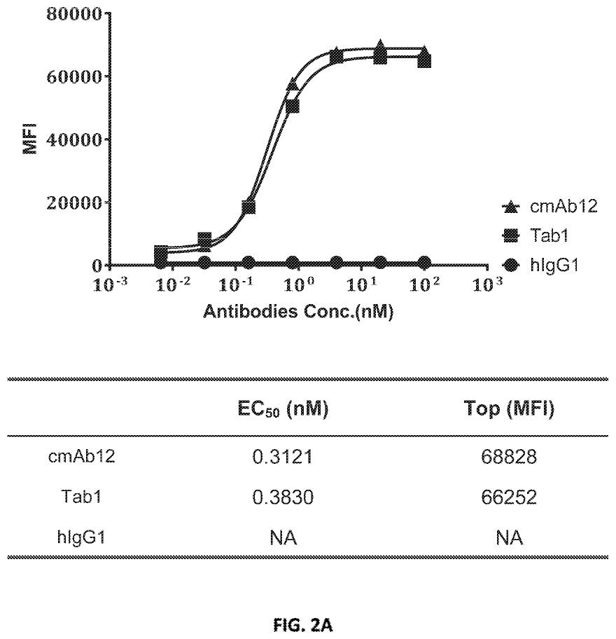

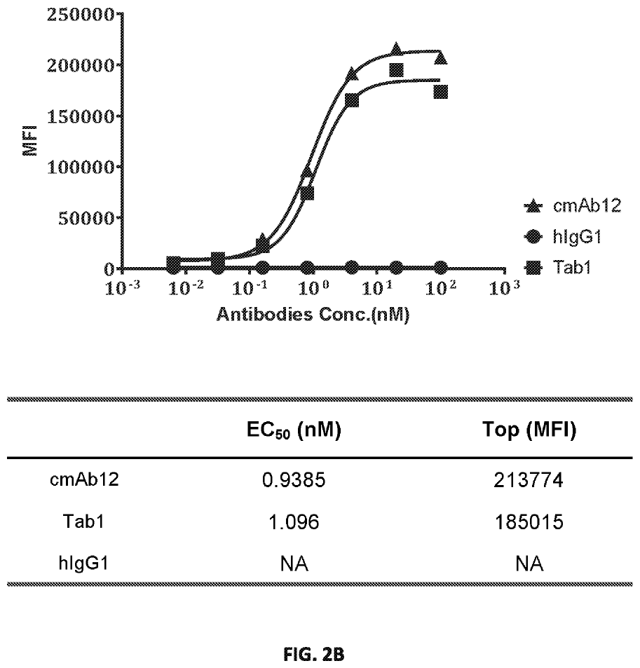

Results and conclusions: As shown by the concentration-MFI (“Median Fluorescence Intensity”) curves in FIGS. 2A-2B, cmAb12 bound to 293F-human ILT7 cells (FIG. 2A) and CHOK1-cynomolgus ILT7 cells (FIG. 2B) in a dose-dependent manner. The EC₅₀ of cmAb12 was 0.3121 nM for 293F-human ILT7 cells and 0.9385 nM for CHOK1-cynomolgus ILT7 cells.

### Example 5: Lack of Cross-Reactivity of cmAb12 to Other LILR Family Members

Methods: The binding specificities of the chimeric antibodies, including cmAb12, for ILT7 over its related 10 LILR superfamily member proteins were evaluated by ELISA. Plates coated with 1 μg/ml LILR superfamily protein as indicated were incubated with chimeric antibodies at 37° C. for 1 hour, followed by the secondary antibody. Detailed OD450 measurements are listed in Table 4.

Results and conclusions: As shown in Table 4, cmAb12 did not specifically bind to other LILR family members (affinity comparable to IgG negative control), demonstrating its high specificity for ILT7.

TABLE 4
Cross reactivity with among LILR family members
ELISA OD 450 nM

| | | LILRA2/ | LILRA3/ | LILRA5/ | LILRA6/ | LILRB1/ | LILRB2/ | LILRB3/ | LILRB4/ | |
|---|---|---|---|---|---|---|---|---|---|---|
| | LILRA1 | ILT1 | ILT6 | ILT11 | ILT8 | ILT2 | ILT4 | ILT5 | ILT3 | LILB5 |
| cmAb12 | 0.1 | 0.2 | 0.1 | 0.1 | 0.1 | 0.1 | 0.3 | 0.1 | 0.1 | 0.1 |

### Example 6: Blocking of CpG-Induced IFNα Production in Human PBMCs by cmAb12

Methods: The activities of the anti-ILT7 chimeric antibodies disclosed herein, including cmAb12, in reducing IFNα release were tested by IFNα release assay. Briefly, PBMC cells (Allcells, frozen) were seeded in assay medium (1640 medium+10% FBS) containing 200 ng/ml IL-2, and pre-incubated with the antibodies as indicated for 6 hours at 4° C., 5% CO₂. ODN2216 solution (CpG-containing DNA) was then added, and the cells were further incubated for 18 hours. Cell culture supernatants were then harvested, and the IFNα levels were measured by human IFNα pan ELISA development kit (HRP).

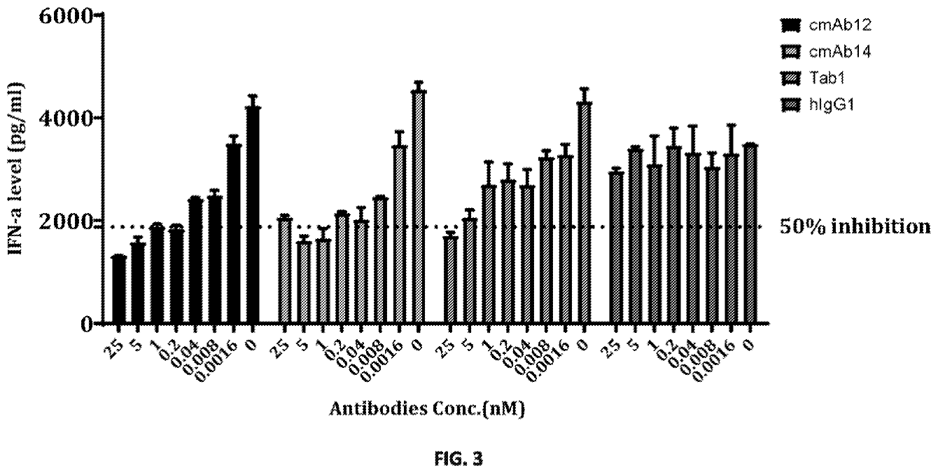

Results and conclusions: As shown in FIG. 3, cmAb12 inhibited IFNα release by CpG-stimulated PBMCs in a dose-dependent manner.

### Example 7: Epitope Binning

Methods: Epitope binning of anti-ILT7 antibodies disclosed herein, including cmAb12, was performed by competitive FACS. In detail, CHOK1 cells expressing human ILT7 were incubated with equal volumes of the reference antibody (Tab1-Alexa488) and the antibody to be tested for 1 hour at 4° C. An isotype antibody hIgG1 was used as negative control. The cells were washed with PBS, and the relative MFIs of different fluorescence signals were analyzed by flow cytometer.

The relative inhibition rate of each tested antibody against Tab1 was calculated as follows: [F(hIgG)−F(tested antibody)]*100%/F(hIgG). F(hIgG) refers to the signal strength of Tab1-Alexa488 in the presence of hIgG; F(tested antibody) refers to the signal strength of Tab1-Alexa488 in the presence of the tested antibody. As such, positive inhibition rates indicate that the tested antibody and Tab1 competitively bind to the same or overlapping epitopes on ILT7, which are grouped in a bin. The results of epitope binning analysis were showed in Table 5.

Results and conclusions: As shown in Table 5, the binding of Tab1-Alexa488 did not influence the binding of cmAb12, indicating that cmAb12 and Tab1 bound to different epitopes on human ILT7.

TABLE 5
Epitope binning measured by competitive FACS

Tab1-Alexa488

| Antibody | MFI | Inhibition (%) |
|---|---|---|
| cmAb12 | 12237.3 | −27 |
| Tab1 | 1391.8 | 86 |
| hIgG1 | 9665.5 | 0 |

### Example 8: Humanization of cmAb12

Methods: For the humanization of cmAb12, IgBLAST from NCBI was used to choose the most appropriate human frameworks for grafting rodent CDRs. Variable regions with high amino acid sequence identities to the rodent variable regions (homology matching or best fit) were used. cmAb12 was humanized by grafting the three VL CDRs into a human VL that was as homologous as possible to the mouse VL. Similarly, the three VH CDRs were grafted into a human VH that was as homologous as possible to the mouse VH. Kabat and Chothia numberings were followed. Furthermore, the sequences of exemplary humanized antibody were listed in Table 3. The binding and function of the humanized antibodies from cmAb12 were evaluated using same experiment procedures described above.

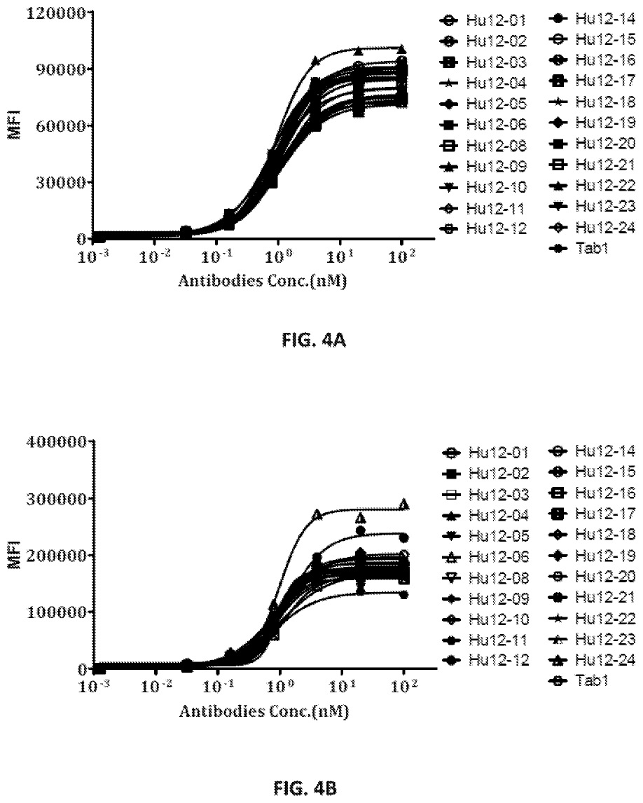

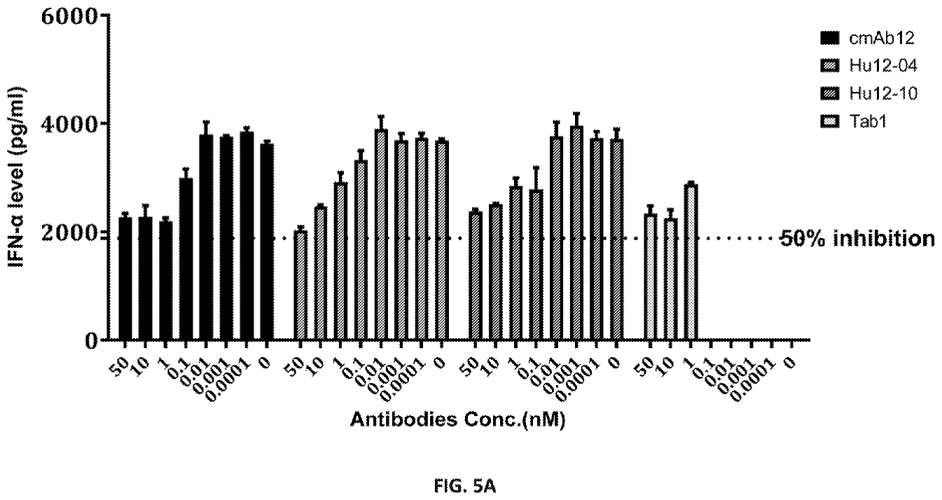

Results and conclusions: As shown in FIGS. 4A-4B, all humanized cmAb12 antibodies maintained high binding affinities to 293F-human ILT7 cells (FIG. 4A) and CHOK1-cynomolgus ILT7 cells (FIG. 4B).

### Example 9: Blocking of CpG-Induced IFNα Production of Humanized Antibodies of cmAb12 in Human PBMCs

Methods: Function of blocking CpG-induced IFN-α secretion was evaluated for four humanized cmAb12 antibodies, cmAb12, positive control antibody (Tab1), and negative control antibody (hIgG1) under the same experiment procedure as described in Example 6.

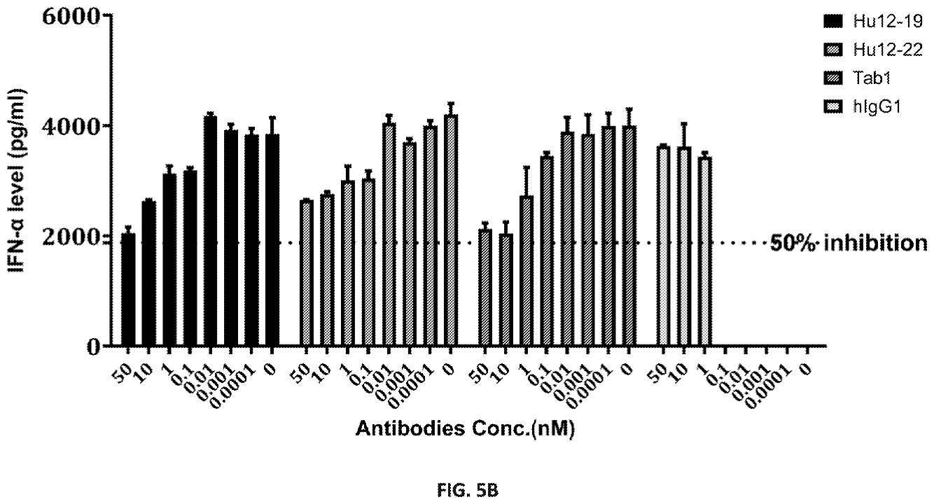

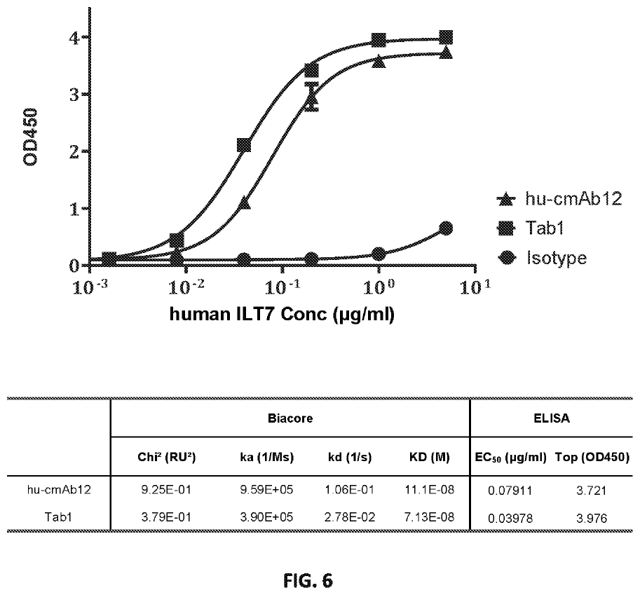

Results and conclusions: As shown in FIGS. 5A and 5B, four humanized antibodies of cmAb12 performed dose-dependent inhibition of IFNα secretion in PBMCs stimulated with CpG.

### Example 10: Binding of hu-cmAb12 to ILT7 Measured by Biacore and ELISA

Methods: The binding affinity of the humanized cmAb12 antibody, hu-cmAb12, was further measured by surface plasmon resonance (SPR) technology with Biacore 8K. As used herein, “hu-cmAb12” refers to a specific antibody clone variant of the Hu12-04 antibody of Examples 8 and 9. The hu-cmAb12 antibody includes a VL having the amino acid sequence of SEQ ID NO: 19, a VH having the amino acid sequence of SEQ ID NO: 26, and a CH having the amino acid sequence of SEQ ID NO: 55. hu-cmAb12 or Tab1 were immobilized on CM-5 chip. The assay was performed at 25° C. and the running buffer was 1×HEPES (10 mM HEPES, 150 mM NaCl, 3 mM EDTA) with 0.005% Tween-20, pH 7.4. Diluted antibodies were captured on the sensor chip through Fc capture method. Human ILT7-his was used as the analyte, and running buffer was used as the dissociation phase. Similarly, ELISA was conducted as described above. An isotype control antibody and reference anti-ILT7 antibody Tab1 were used as controls.

Results and conclusions: The hu-cmAb12 antibody bound to human ILT7 with a K_{D} of 111 nM as measured by Biacore (data not shown) and an EC₅₀ of 0.07911 μg/ml as measured by ELISA (FIG. 6), both comparable to Tab1.

### Example 11: Binding of hu-cmAb12 to ILT7-Expressing Cells Measured by Flow Cytometry Analysis

Methods: Both the CHOK1-cynomolgus ILT7 cells and 293F-human ILT7 cells were seeded, resuspended in a solution comprising the hu-cmAb12 antibody, and incubated at 4° C. for 1 hr. Cells were washed with cold FACS buffer (PBS+2% FBS) by centrifuging, followed by addition of secondary antibodies, and incubation at 4° C. for 1 hour. An isotype control antibody and reference anti-ILT7 antibody Tab1 were used as negative and positive controls. The concentration-MFI curves of FACs were then determined.

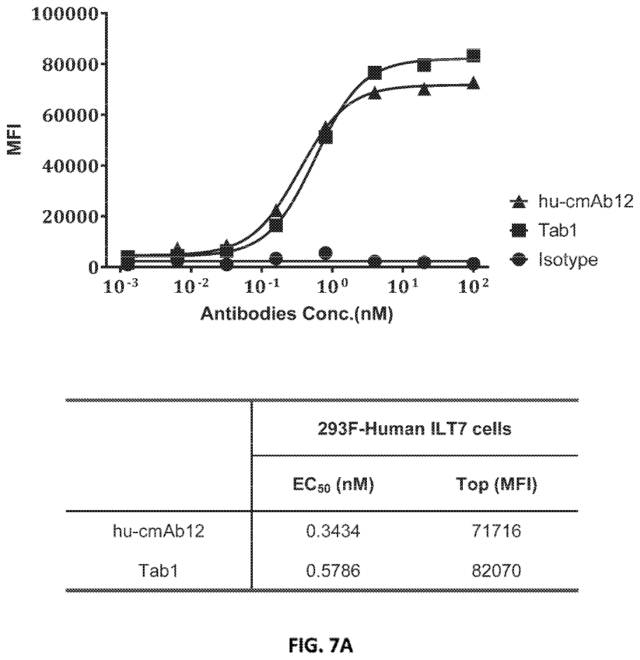

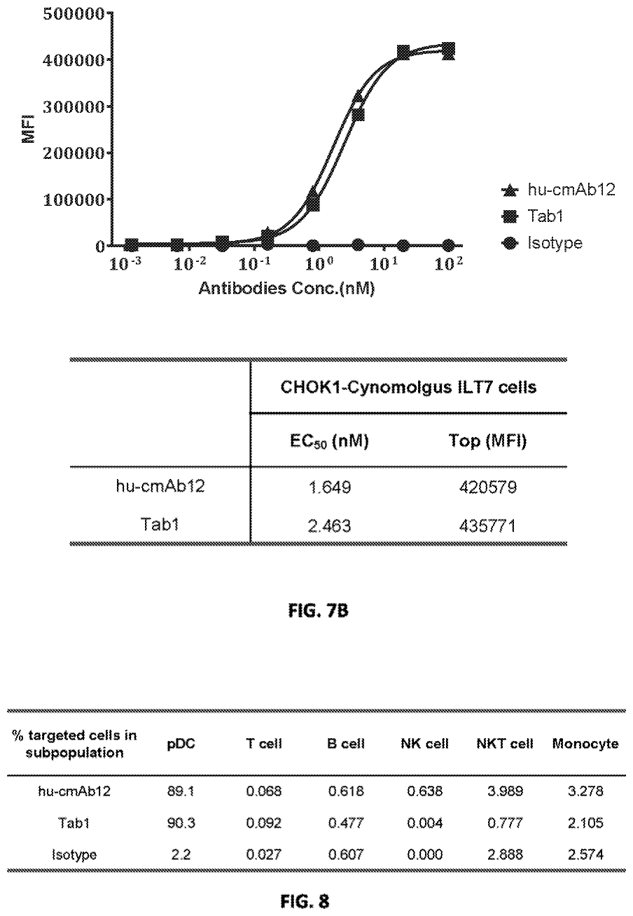

Results and conclusions: Representative results are shown in FIGS. 7A-7B, which show that the hu-cmAb12 antibody bound to 293F-human ILT7 cells (FIG. 7A) and CHOK1-cynomolgus ILT7 cells (FIG. 7B) with an EC₅₀ of 0.3434 nM and 1.649 nM respectively, comparable to Tab1.

### Example 12: Binding of hu-cmAb12 to Human PBMCs

Methods: The hu-cmAb12 antibody was conjugated with fluorescent dye Alexa488. and PBMC cells (Allcells, frozen) were seeded in assay medium (1640 medium+10% FBS). All the cells were stained with live/dead dye first, then the specific binding of hu-cmAb12 was detected in T cells, B cells, natural killer cell, natural killer T cell, monocytes and plasmacytoid dendritic cells. In particular, plasmacytoid dendritic cells were identified as: CD11c low, HLA-DR positive, CD123 positive.

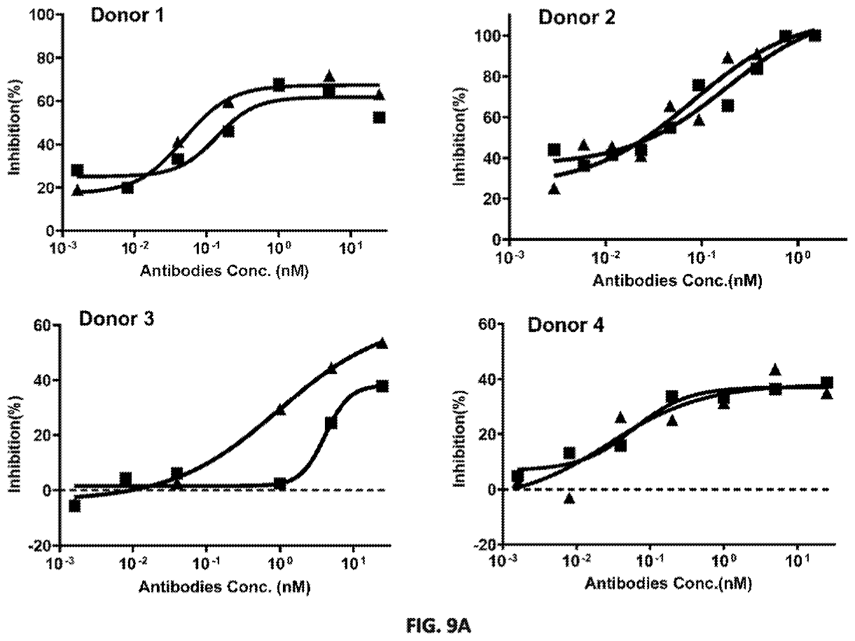

Results and conclusions: Representative results are provided in FIG. 8. As shown, similar to Tab1, the hu-cmAb12 antibody specifically bound to pDCs in human PBMCs, but not to T cells, B cells, NK cells, NKT cells, or monocytes.

### Example 13: Blocking CpG-Induced IFNα Production in Human PBMCs by hu-cmAb12

Methods: The hu-cmAb12 antibody was also tested for its activity in reducing IFNα release by IFNα release assay using the same experiment procedures described above. PBMCs obtained from 4 different donors were tested.

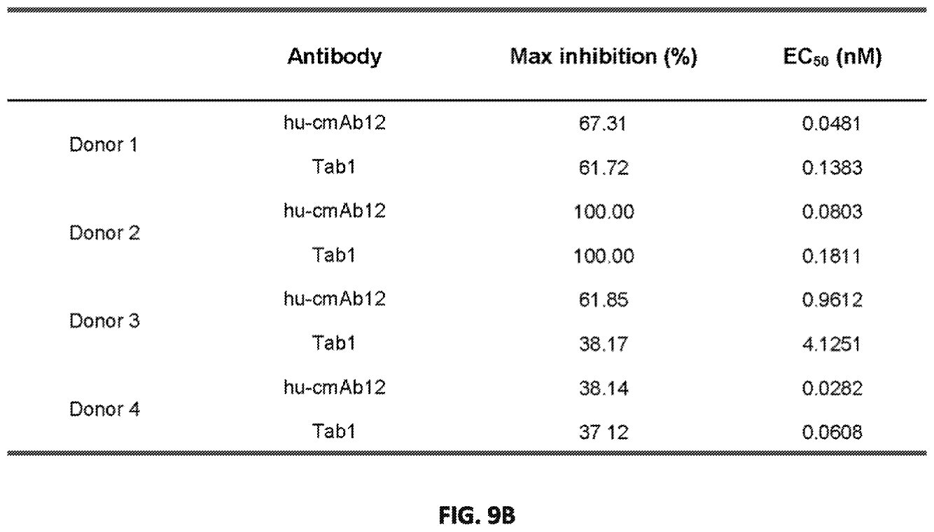

Results and conclusions: Representative results are provided in FIGS. 9A-9B. As shown, hu-cmAb12 strongly inhibited IFNα release in CpG-stimulated PBMCs obtained from all 4 donors. Compared with Tab1, hu-cmAb12 exhibited lower EC₅₀ and higher inhibition rate.

### Example 14: NK Cell-Dependent ADCC Activity of hu-cmAb12

Methods: 293F-human ILT7 cells were used as the target cell. After being labelled with DELFIA BATDA Reagent (Perkin Elmer, ADO 116) at 37° C. for 20 minutes, the cells were washed, and resuspended in assay medium (RPMI1640 medium, no phenol red with 2% FBS). Meanwhile, NK effector cells (NK-92 CD16a 176V) were also resuspended in assay medium. The target cells and effector cells were then mixed and seeded at the number ratio of 5:1. Subsequently, the hu-cmAb12 antibody was added, and the plates were incubated at 37° C., 5% C02 for 4 hours. Supernatants were transferred to DELFIA® yellow 96 well plates (Perkin Elmer, cat #AAAND-0001) pre-filled with Europium Solution, and fluorescence absorbance at 615 nm was measured. The ADCC results were demonstrated by % Cytotoxicity.

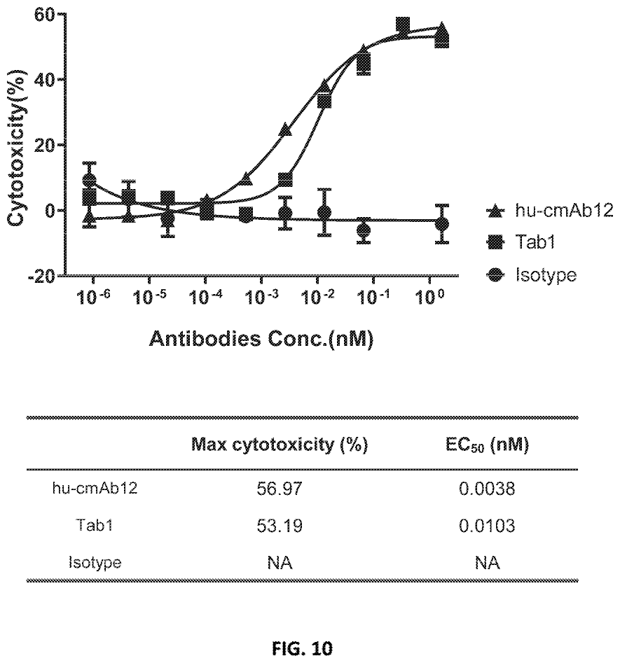

Results and conclusions: Representative results are provided in FIG. 10. As shown, hu-cmAb12 depleted ILT7-expressing cells through NK-dependent ADCC activity with an EC₅₀ of 0.0038 nM, lower than that of Tab1 (0.0103 nM).

### Example 15: Neutrophil-Dependent ADCC Activity of hu-cmAb12

Methods: 293F-human ILT7 cells were used as target cells. The cells were washed and resuspended in assay medium (RPMI1640 medium, no phenol red with 2% FBS). Meanwhile, neutrophils were isolated from whole blood using density gradient centrifugation and resuspended in the assay medium containing GM-CSF (50 U/mL). The target cells and effector cell were mixed and seeded at the number ratio of 80:1. Subsequently, the hu-cmAb12 antibody was added, and the plates were incubated at 37° C., 5% C02 for 3 hours. Released LDH in the supernatant was detected using the LDH assay kit. The killing effects of neutrophils on target cells were demonstrated by % Cytotoxicity.

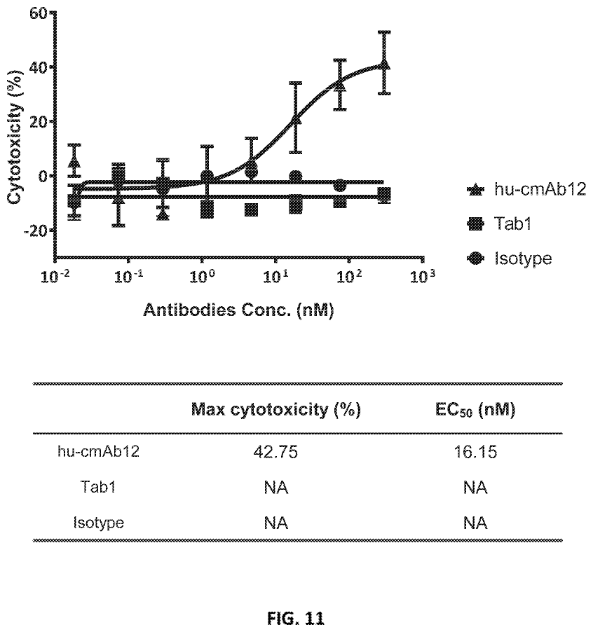

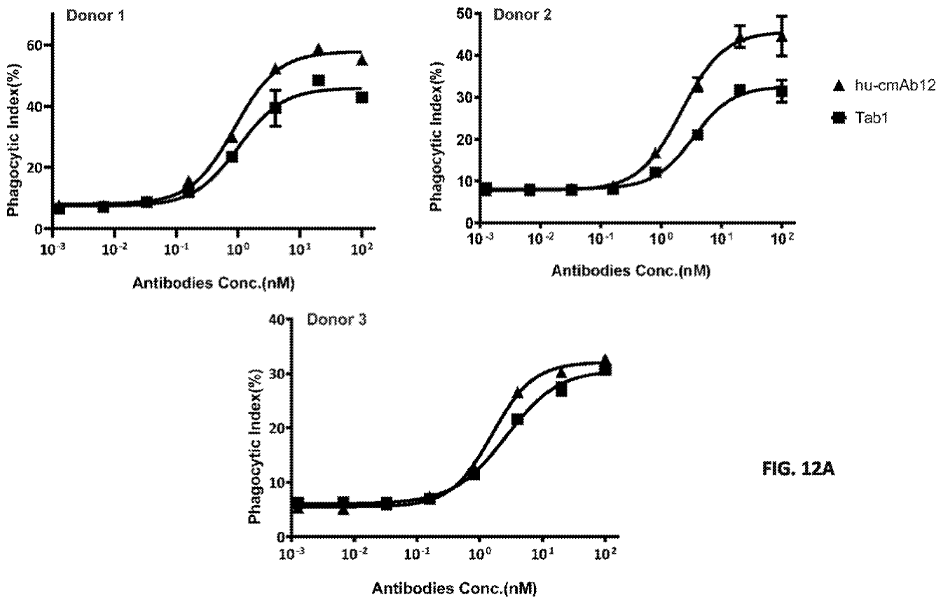

Results and conclusions: Representative results are provided in FIG. 11. As shown, hu-cmAb12 depleted ILT7-expressing cells via neutrophil-dependent ADCC activity with an EC₅₀ of 16.15 nM. By contrast, Tab1 lacked neutrophil-dependent ADCC activity.

### Example 16: Macrophage-Dependent ADCP Activity of hu-cmAb12

Methods: PBMCs were obtained from three different donors, seeded for 2 hours in in 1640 media without FBS, then un-adherent cells were cultured with the inducing medium (1640+10% FBS+20 ng/mL rhuM-CSF). Half of the inducing medium was replaced with fresh inducing medium every 2 days. On day 8, monocyte-derived macrophages (MDM) were digested and pre-seeded as effector cells. 293F-human ILT7 cells were used as target cells. The cells were labeled with 2 μM CFSE in PBS at 37° C. for 8 min, washed, and resuspended. Cultured MDMs were added with the hu-cmAb12, antibody followed by the same amount of target cells, and incubated for 4 hours at 37° C. All cells were then detached and stained with APC mouse anti-hCD11b for flow cytometry analysis. Phagocytosis % reflected the percentage of MDMs which had engulfed target cells. Phagocytosis % curves of ADCP were showed.

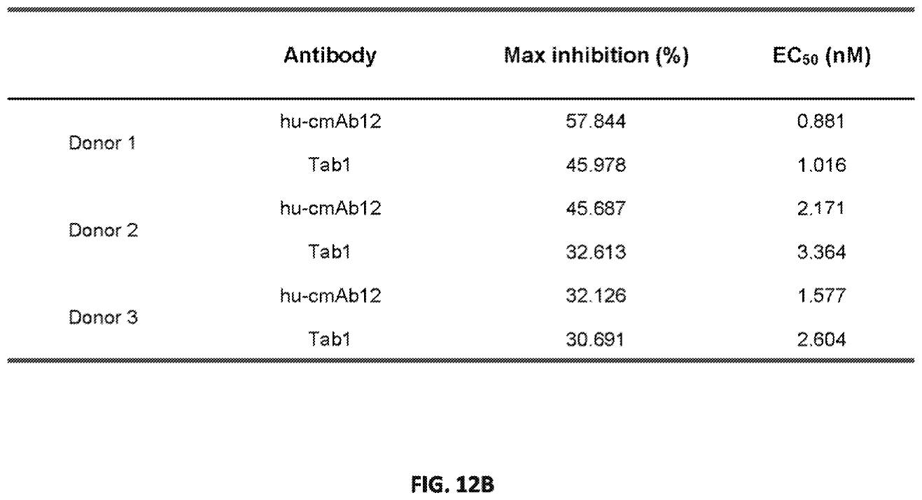

Results and conclusions: Representative results are provided in FIGS. 12A-12B. As shown, using PBMCs from three different donors, hu-cmAb12 depleted ILT7-expressing cells through macrophage-dependent ADCP activity with an EC₅₀ of 0.881 nM (Donor 1), 2.171 nM (Donor 2), and 1.577 nM (Donor 3), and a maximum phagocytic index of 57.844% (Donor 1), 45.687% (Donor 2) and 32.126% (Donor 3), respectively, exhibiting better phagocytosis activity than Tab1.

### Example 17: Specific pDC Depletion by hu-cmAb12 in Humanized Mice and Cynomolgus Monkeys

Methods: 18 NOG-EXL mice with at least 8-week immune reconstitution were utilized. 5 μg FLT3L-protein was injected intravenously into mice three times a week for 2 weeks. Two days after the last injection of FLT3L was defined as the day before Day 0 (Day −1). Then, blood of all mice was sampled and proportions of T cells, B cells, NK cells, pDCs and monocytes were detected by flow cytometry analysis. After each mouse received 5×104 human NK cells intravenously, an isotype control antibody hIgG1 at a dose of 5 mg/kg or the hu-cmAb12 antibody at a dose of 1 or 5 mg/kg were injected intraperitoneally on Day 0. Peripheral blood of all mice was sampled and proportions of T cells, B cells, NK cells, pDCs and monocytes were detected by flow cytometry analysis 24 h post antibody injection. In particular, human pDCs in the mice were identified as: hCD45 positive, mouse CD45 negative, CD3 negative, CD19 negative, CD14 negative, HLA-DR positive, CD123 positive.

Cynomolgus monkeys were also utilized. Specifically, vehicle or the hu-cmAb12 antibody at a dose of 30 mg/kg were injected intravenously on Day 0. Peripheral blood of all monkeys was sampled and proportions of T cells, B cells, NK cells, pDCs and monocytes were detected by flow cytometry analysis at Day 0 before antibody injection, Day 1 (24 h post antibody injection), Day 7 (168 h post antibody injection) and Day 28 (672 h post antibody injection). In particular, pDCs in cynomolgus monkeys were identified as: CD45 positive, CD19 negative, CD3 negative, CD159a negative, HLA-DR positive, CD14 negative, CD1c negative, CD123 positive.

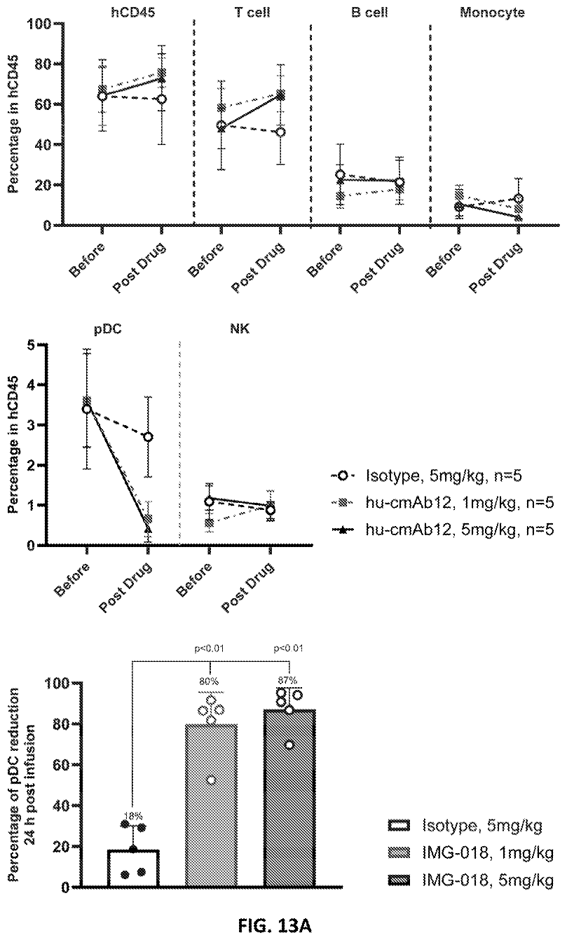

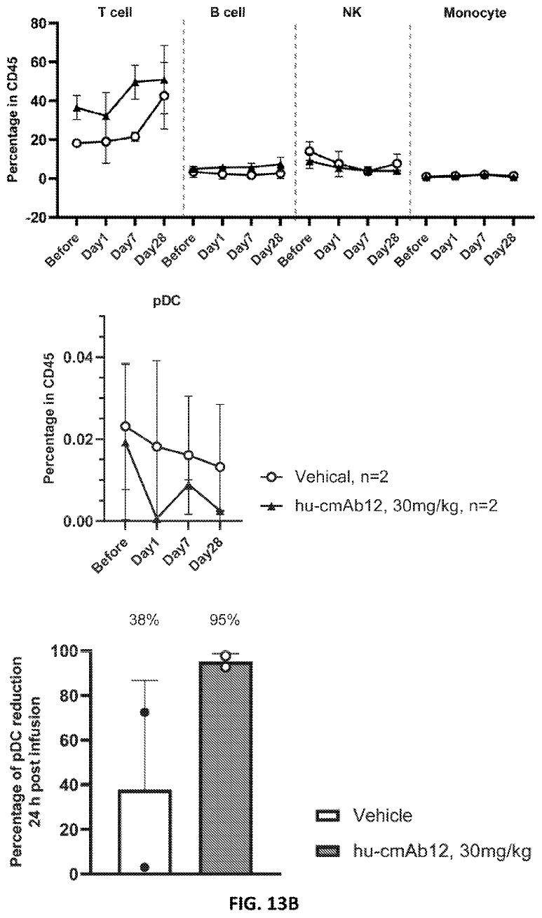

Results and conclusions: Representative results are provided in FIGS. 13A-13B. As shown, the hu-cmAb12 antibody specifically and rapidly depleted pDCs in humanized mice and cynomolgus monkeys 24 h post infusion, while other cells remained unaffected. In humanized mice, the hu-cmAb12 antibody at doses of 1 and 5 mg/kg reduced pDCs by 80% and 87% 24 h post infusion, respectively, compared with 18% reduction in the IgG1 isotype group (FIG. 13A). In cynomolgus monkeys, the hu-cmAb12 antibody reduced pDCs by 95% 24 h post infusion, compared with 38% reduction in the control (FIG. 13B).

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

All patents, patent publications, patent applications, journal articles, books, technical references, and the like discussed in the instant disclosure are incorporated herein by reference in their entirety for all purposes.

In the foregoing description, numerous specific details are set forth to provide a more thorough understanding of the present disclosure. However, it will be apparent to one of skill in the art that the embodiments described in this disclosure may be practiced without one or more of these specific details. In other instances, well-known features and procedures well known to those skilled in the art have not been described in order to avoid obscuring the disclosure. Embodiments of the disclosure have been described for illustrative and not restrictive purposes. Although the present disclosure is described primarily with reference to specific embodiments, it is also envisioned that other embodiments will become apparent to those skilled in the art upon reading the present disclosure, and it is intended that such embodiments be contained within the present inventive methods. Accordingly, the present disclosure is not limited to the embodiments described above or depicted in the drawings, and various embodiments and modifications can be made without departing from the scope of the claims below.

1. An antibody or antigen-binding fragment thereof that specifically binds human ILT7, the antibodies or antigen-binding fragments comprising:
(1) as defined by Kabat,
(a) a light chain variable region (VL) comprising VL CDR1, VL CDR2, and VL CDR3 having the amino acid sequences of SEQ ID NOs: 11, 12, and 13, respectively; or a variant thereof having up to about 5 amino acid substitutions, additions, and/or deletions in the VL CDRs; and/or
(b) a heavy chain variable region (VH) comprising VH CDR1, VH CDR2, and VH CDR3 having the amino acid sequences of SEQ ID NOs: 14, 15, and 16, respectively; or a variant thereof having up to about 5 amino acid substitutions, additions, and/or deletions in the VH CDRs; or
(2) as defined by Chothia,
(a) a VL comprising VL CDR1, VL CDR2, and VL CDR3 having the amino acid sequences of SEQ ID NOs: 11, 12, and 13, respectively; or a variant thereof having up to about 5 amino acid substitutions, additions, and/or deletions in the VL CDRs; and/or
(b) a VH comprising VH CDR1, VH CDR2, and VH CDR3 having the amino acid sequences of SEQ ID NOs: 17, 18, and 16, respectively; or a variant thereof having up to about 5 amino acid substitutions, additions, and/or deletions in the VH CDRs.

2. The antibody or antigen-binding fragment of claim 1, comprising VL CDR1, VL CDR2, VL CDR3, VH CDR1, VH CDR2 and VH CDR3 having the amino acid sequences of SEQ ID NOs: 11, 12, 13, 14, 15, and 16, respectively, as defined by Kabat.

3. The antibody or antigen-binding fragment of claim 1, comprising VL CDR1, VL CDR2, VL CDR3, VH CDR1, VH CDR2 and VH CDR3 having the amino acid sequences of SEQ ID NOs: 11, 12, 13, 17, 18, and 16, respectively, as defined by Chothia.

4. An antibody or antigen-binding fragment thereof that specifically binds human ILT7, the antibodies or antigen-binding fragments comprising:
(a) a VL having at least 85%, at least 90%, at least 95%, at least 98%, or 100% sequence identity to the amino acid sequence of SEQ ID NO: 9; and/or
(b) a VH having at least 85%, at least 90%, at least 95%, at least 98%, or 100% sequence identity to the amino acid sequence of SEQ ID NO: 10.

5. The antibody or antigen-binding fragment of claim 4, comprising a VL and a VH having the amino acid sequences of SEQ ID NOs: 9 and 10, respectively.

6. An antibody or antigen-binding fragment thereof that specifically binds human ILT7, the antibody or antigen-binding fragment comprising
(a) a VL comprising VL CDR1, VL CDR2, and VL CDR3 from a VL having the amino acid sequence of SEQ ID NO: 9; and/or
(b) a VH comprising VH CDR1, VH CDR2, and VH CDR3 from a VH having the amino acid sequence of SEQ ID NO: 10.

7. The antibody or antigen-binding fragment of any one of claims 1 to 6, wherein the antibody or antigen-binding fragment is a chimeric antibody or antigen-binding fragment, a humanized antibody or antigen-binding fragment, or a human antibody or antigen-binding fragment.

8. The antibody or antigen-binding fragment of claim 7, wherein the antibody or antigen-binding fragment is a humanized antibody or antigen-binding fragment.

9. The antibody or antigen-binding fragment of claim 8, comprising:
(a) a VL having at least 85%, at least 90%, at least 95%, at least 98%, or 100% sequence identity to the amino acid sequence of any one of SEQ ID NOs: 19-22; and/or
(b) a VH having at least 85%, at least 90%, at least 95%, at least 98%, or 100% sequence identity to the amino acid of any one of SEQ ID NOs: 23-28.

10. The antibody or antigen-binding fragment of claim 9, comprising:
(a) a VL having at least 85%, at least 90%, at least 95%, at least 98%, or 100% sequence identity to the amino acid sequence of SEQ ID NO: 19; and
(b) a VH having at least 85%, at least 90%, at least 95%, at least 98%, or 100% sequence identity to the amino acid sequence of SEQ ID NO: 26.

11. The antibody or antigen-binding fragment of claim 9, comprising:
(a) a VL having at least 85%, at least 90%, at least 95%, at least 98%, or 100% sequence identity to the amino acid sequence of SEQ ID NO: 19, the VL comprising, as defined by Kabat or Chothia, VL CDR1, VL CDR2, and VL CDR3 having the amino acid sequences of SEQ ID NOs: 11, 12, and 13, respectively; and
(b) a VH having at least 85%, at least 90%, at least 95%, at least 98%, or 100% sequence identity to the amino acid sequence of SEQ ID NO: 26, the VH comprising (1) as defined by Kabat, VH CDR1, VH CDR2, and VH CDR3 having the amino acid sequences of SEQ ID NOs: 14, 15, and 16, respectively, or (2) as defined by Chothia, VH CDR1, VH CDR2, and VH CDR3 having the amino acid sequences of SEQ ID NOs: 17, 18, and 16, respectively.

12. The antibody or antigen-binding fragment of claim 9, comprising a VL and a VH having the amino acid sequences of (1) SEQ ID NOs: 19 and 23, respectively; (2) SEQ ID NOs: 19 and 24, respectively; (3) SEQ ID NOs: 19 and 25, respectively; (4) SEQ ID NOs: 19 and 26, respectively; (5) SEQ ID NOs: 19 and 27, respectively; (6) SEQ ID NOs: 19 and 28, respectively; (7) SEQ ID NOs: 20 and 23, respectively; (8) SEQ ID NOs: 20 and 24, respectively; (9) SEQ ID NOs: 20 and 25, respectively; (10) SEQ ID NOs: 20 and 26, respectively; (11) SEQ ID NOs: 20 and 27, respectively; (12) SEQ ID NOs: 20 and 28, respectively; (13) SEQ ID NOs: 21 and 23, respectively; (14) SEQ ID NOs: 21 and 24, respectively; (15) SEQ ID NOs: 21 and 25, respectively; (16) SEQ ID NOs: 21 and 26, respectively; (17) SEQ ID NOs: 21 and 27, respectively; (18) SEQ ID NOs: 21 and 28, respectively; (19) SEQ ID NOs: 22 and 23, respectively; (20) SEQ ID NOs: 22 and 24, respectively; (21) SEQ ID NOs: 22 and 25, respectively; (22) SEQ ID NOs: 22 and 26, respectively; (23) SEQ ID NOs: 22 and 27, respectively; or (24) SEQ ID NOs: 22 and 28, respectively.

13. The antibody or antigen-binding fragment of claim 12, comprising a VL and a VH having the amino acid sequences of SEQ ID NOs: 19 and 26, respectively.

14. The antibody or antigen-binding fragment of any one of claims 1 to 13, wherein the antibody or antigen-binding fragment is a Fab, a Fab′, a F(ab′)2, a Fv, a scFv, a (scFv)2, a single domain antibody (sdAb), or a heavy chain antibody (HCAb).

15. The antibody or antigen-binding fragment of any one of claims 1 to 13, wherein the antibody or antigen-binding fragment is an IgG1 antibody, an IgG2 antibody, an IgG3 antibody, or an IgG4 antibody.

16. The antibody or antigen-binding fragment of claim 15, wherein the antibody is an IgG1 antibody.

17. The antibody or antigen-binding fragment of claim 16, comprising a light chain constant region (CL) having at least 85% sequence identity to kappa CL (Cκ; SEQ ID NO: 29).

18. The antibody or antigen-binding fragment of claim 16, comprising a light chain constant region (CL) having at least 85% sequence identity to lambda CL (Cλ; SEQ ID NO: 30).

19. The antibody or antigen-binding fragment of claim 16, comprising a heavy chain constant region (CH) having at least 85% sequence identity to the amino acid sequence of any one of SEQ ID NOs: 31 and 40-44.

20. The antibody or antigen-binding fragment of any one of claims 16 to 19, wherein the heavy chain constant region (CH) of the IgG1 antibody comprises a wildtype IgG1 CH, or comprises at least one amino acid mutation that enhances ADCC (antibody-dependent cellular cytotoxicity) or ADCP (antibody-dependent cellular phagocytosis) of the antibody.

21. The antibody or antigen-binding fragment of claim 20, wherein the CH region of the IgG1 antibody has an amino acid substitution at L234, L235, G236, S239, F243, H268, D270, R292, S298, Y300, V305, K326, A330, I332, E333, K334, P396, or any combination thereof, numbered according to the EU Index.

22. The antibody or antigen-binding fragment of claim 20, wherein the CH region of the IgG1 antibody has an amino acid substitution that is L234Y, L235Q, L235V, G236A, G236W, S239D, S239M, F243L, H268D, D270E, R292P, S298A, Y300L, V305I, K326D, A330M, A330L, I332E, E333A, K334A, K334E, or P396L, or any combination thereof, numbered according to the EU Index.

23. The antibody or antigen-binding fragment of claim 20, wherein the CH region of the IgG1 antibody is modified by amino acid substitutions that are (i) S298A, E333A, and K334A; (ii) S239D and I332E; (iii) S239D, A330L, and I332E; (iv) G236A; (v) G236A, S239D, and I332E; (vi) G236A, A330L, and I332E; (vii) G236A, S239D, A330L, and I332E; (viii) F243L, R292P, Y300L, V305I, and P396L; (ix) L235V, F243L, R292P, Y300L, and P396L; (x) L234Y, L235Q, G236W, S239M, H268D, D270E, and S298A; or (xi) D270E, K326D, A330M, and K334E, numbered according to the EU Index.

24. The antibody or antigen-binding fragment of claim 23, wherein the CH region has the amino acid sequence of any one of SEQ ID NOs: 45-64.

25. The antibody or antigen-binding fragment of claim 1, comprising:
a VL having the amino acid sequence of SEQ ID NO: 19;
a VH having the amino acid sequence of SEQ ID NO: 26; and
a CH having the amino acid sequence of SEQ ID NO: 55

26. The antibody or antigen-binding fragment of claim 1, comprising:
a VL having the amino acid sequence of SEQ ID NO: 19;
a VH having the amino acid sequence of SEQ ID NO: 26;
a CL having the amino acid sequence of SEQ ID NO: 30; and
a CH having the amino acid sequence of SEQ ID NO: 55.

27. The antibody or antigen-binding fragment of any one of claims 16 to 26, wherein the Fc region of the IgG1 antibody is afucosylated.

28. An antibody or antigen-binding fragment thereof that competes with the antibody or antigen-binding fragment of any one of claims 1 to 27 for binding to human ILT7.

29. The antibody or antigen-binding fragment of any one of claims 1 to 28, wherein the antibody or antigen-binding fragment is a bispecific antibody or a multispecific antibody.

30. The antibody or antigen-binding fragment of any one of claims 1 to 29, wherein the antibody or antigen-binding fragment is a monoclonal antibody or antigen-binding fragment.

31. The antibody or antigen-binding fragment of any one of claims 1 to 30, wherein the antibody or antigen binding fragment:
(1) binds to human ILT7 with a K_{D} of 500 nM or less, as measured by SPR;
(2) does not specifically bind to LILR family member LILRA1, LILRA2/ILT1, LILRA3/ILT6, LILRA5/ILT11, LILRA6/ILT8, LILRB1/ILT2, LILRB2/ILT4, LILRB3/ILT5, LILRB4/ILT3, or LILB5;
(3) inhibits interferon alpha (IFNα) release by peripheral blood mononuclear cells (PBMCs);
(4) selectively binds to plasmacytoid dendritic cells (pDCs) in human PBMC;
(5) exhibits natural killer cell (NK)-dependent ADCC activity against ILT7-expressing cells;
(6) exhibits neutrophil-dependent ADCC activity against ILT7-expressing cells; or
(7) exhibits macrophage-dependent ADCP activity against ILT7-expressing cells; or any combination of (1)-(7).

32. An antibody or antigen-binding fragment thereof that specifically binds the protease domain of human ILT7, wherein the antibody or antigen binding fragment:
(1) binds to human ILT7 with a K_{D} of 500 nM or less, as measured by SPR;
(2) does not specifically bind to LILR family member LILRA1, LILRA2/ILT1, LILRA3/ILT6, LILRA5/ILT 11, LILRA6/ILT8, LILRB1/ILT2, LILRB2/ILT4, LILRB3/ILT5, LILRB4/ILT3, or LILB5;
(3) inhibits IFNα release by PBMCs;
(4) selectively binds to pDCs in human PBMCs;
(5) exhibits NK-dependent ADCC activity against ILT7-expressing cells;
(6) exhibits neutrophil-dependent ADCC activity against ILT7-expressing cells; or
(7) exhibits macrophage-dependent ADCP activity against ILT7-expressing cells; or any combination of (1)-(7).

33. The antibody or antigen-binding fragment of claim 31 or 32, wherein the antibody or antigen-binding fragment:
(1) inhibits IFNα release by CpG-stimulated PBMCs in vitro with an EC50 of 1 nM or less;
(2) exhibits NK-dependent ADCC activity against ILT7-expressing cells with an EC50 of 0.01 nM or less;
(3) exhibits neutrophil-dependent ADCC activity against ILT7-expressing cells with an EC50 of 100 nM or less;
(4) exhibits macrophage-dependent ADCP activity against ILT7-expressing cells with an EC50 of 10 nM or less; or
(5) exhibits macrophage-dependent ADCP activity against ILT7-expressing cells with a maximum phagocytic index of 20% or higher; or any combination of (1)-(5).

34. The antibody or antigen-binding fragment of claim 33, wherein the antibody or antigen-binding fragment:
(1) inhibits IFNα release by PBMCs with an EC50 ranging from 0.01 nM to 0.1 nM;
(2) exhibits NK-dependent ADCC activity against ILT7-expressing cells with an EC50 ranging from 0.001 nM to 0.01 nM;
(3) exhibits neutrophil-dependent ADCC activity against ILT7-expressing cells with an EC50 ranging from 1 nM to 50 nM;
(4) exhibits macrophage-dependent ADCP activity against ILT7-expressing cells with an EC50 ranging from 0.5 nM to 5 nM; or
(5) exhibits macrophage-dependent ADCP activity against ILT7-expressing cells with a maximum phagocytic index ranging from 20% to 80%; or any combination of (1)-(5).

35. The antibody or antigen-binding fragment of any one of claims 31 to 34, wherein the antibody or antigen-binding fragment exhibits neutrophil-dependent ADCC activity.

36. A polynucleotide encoding a polypeptide of the antibody or antigen-binding fragment of any one of claims 1 to 35.

37. A vector comprising the polynucleotide of claim 36.

38. A host cell comprising the polynucleotide of claim 36, or the vector of claim 37.

39. The host cell of claim 38, wherein the host cell:
(1) overexpresses N-acetylglucosaminyltransferase III (GnTIII),
(2) lacks a-1,6-fucosyltransferase (FUT8), or
(3) has a low fucose content, or any combination of (1)-(3).

40. A method of making an antibody or antigen-binding fragment thereof that specifically binds human ILT7, the method comprising culturing the host cell of claim 38 or 39 in a culture under conditions that allow expression of the antibody or antibody fragment.

41. The method of claim 40, further comprising isolating the antibody from the culture.

42. A pharmaceutical composition comprising a therapeutically effective amount of the antibody or antigen-binding fragment of any one of claims 1 to 35, and a pharmaceutically acceptable carrier.

43. A method of reducing Type I interferon (IFN) in a subject in need thereof, the method comprising administering to the subject a therapeutically effective amount of the antibody or antigen-binding fragment of any one of claims 1 to 35.

44. The method of claim 43, wherein the Type I interferon is IFNα.

45. A method of suppressing or depleting pDCs in a subject in need thereof, the method comprising administering to the subject a therapeutically effective amount of the antibody or antigen-binding fragment of any one of claims 1 to 35.

46. A method of reducing autoimmunity in a subject in need thereof, the method comprising administering to the subject an effective amount of the antibody or antigen-binding fragment of any one of claims 1 to 35.

47. The method of any one of claims 43 to 46, wherein the subject has an autoimmune disease.

48. A method of treating an autoimmune disease associated with Type I IFN or pDCs in a subject in need thereof, the method comprising administering to the subject a therapeutically effective amount of the antibody or antigen-binding fragment of any one of claims 1 to 35.

49. The method of claim 47 or 48, wherein the autoimmune disease is systemic lupus erythematosus (SLE).

50. The method of any one of claims 43 to 49, further comprising administering an additional therapy to the subject.

51. The method of any one of claims 43 to 50, wherein the subject is a human.

52. Use of the antibody or antigen-binding fragment of any one of claims 1 to 35 in reducing Type I IFN.

53. Use of the antibody or antigen-binding fragment of any one of claims 1 to 35 for preparation of a medicament for reducing Type I IFN.

54. The use of claim 52 or 53, wherein the Type I IFN is IFNα.

55. Use of the antibody or antigen-binding fragment of any one of claims 1 to 35 in suppressing or depleting pDCs.

56. Use of the antibody or antigen-binding fragment of any one of claims 1 to 35 for preparation of a medicament for suppressing or depleting pDCs.

57. Use of the antibody or antigen-binding fragment of any one of claims 1 to 35 in reducing autoimmunity.

58. Use of the antibody or antigen-binding fragment of any one of claims 1 to 35 for preparation of a medicament for reducing autoimmunity.

59. Use of the antibody or antigen-binding fragment of any one of claims 1 to 35 in treating an autoimmune disease associated with Type I IFN or pDCs.

60. Use of the antibody or antigen-binding fragment of any one of claims 1 to 35 for preparation of a medicament for treating an autoimmune disease associated with Type I IFN or pDCs.

61. The use of claim 59 or 60, wherein the autoimmune disease is SLE.
